(19) United States
(12) Patent Application Publication
(10) Pub. No.: US 2026/0241181 A1
(43) Pub. Date: Aug. 20, 2026

(54) SURGICAL TECHNIQUES FOR IMPLANTING ELECTRODE LEADS

(71) Applicant: Amber Therapeutics Holdings Limited, Didcot (GB)

(72) Inventors: Stefan de Wachter, Antwerp (BE); Charles Knowles, London (GB); Nazir-Ahmed Karbanee, York (GB); Gary Moore, Wetherby (GB)

(21) Appl. No.: 19/544,857

(22) Filed: Feb. 19, 2026

Related U.S. Application Data
(60) Provisional application No. 63/761,087, filed on Feb. 20, 2025.

(51) Int. Cl. A61N 1/378; A61M 25/06; A61N 1/05

(52) U.S. Cl. CPC A61N 1/3787; A61M 25/0662; A61N 1/0551

(57) ABSTRACT

Provided herein are devices, systems, and methods for implanting one or more electrode leads anchored at target tissue to deliver a stimulation to treat a disease or condition of one or more tissues. The orientation of the target tissue may be standardized by adjusting the operating table based on one or more landmarks of an x-ray image. A stimulating or marking needle assembly used to verify the placement of the one or more electrode leads may comprise a removable stylet and a needle grip defining a connection region for coupling to external device. A plurality of stimulating needles may be inserted into a patient with a needle guide to determine an optimal needle location for lead placement. Also provided herein are devices, systems, and methods for implanting one or more electrode leads without the use of a guidewire.

### INCORPORATION BY REFERENCE TO ANY PRIORITY APPLICATIONS

This application claims the benefit of U.S. Provisional Application No. 63/761087, filed Feb. 20, 2025, the entire disclosure of which is hereby incorporated by reference herein in its entirety.

### BACKGROUND

### Field

The present disclosure generally relates to systems and methods for implanting one or more electrode leads and anchoring the electrode leads to target tissue.

### Description of the Related Art

Electrical stimulation has been used to treat incontinence, pelvic pain, sexual dysfunction, or other pelvic conditions. In particular, electrodes may be implanted in a pelvic region of the subject to provide electrical stimulation as clinical treatment and/or condition management. Approaches to provide implanted electrodes for treatment and/or condition management are limited by the ability to fix the implanted location of an electrode lead in a pelvic region relative to the tissue due to the forces acted upon the electrode lead by surrounding tissue during implantation and when a subject with an implanted electrode lead moves or ambulates.

### SUMMARY

Fixing the position of an implanted electrode lead at a target one or more nerves and/or tissue adjacent the one or more nerves may improve the robustness of delivering spatially controlled stimulation to the one or more nerves to treat and/or manage a condition, for example a pelvic condition. In some cases, the pelvic condition comprises urinary incontinence, fecal incontinence, pain, sexual dysfunction, or any combination thereof.

Although several embodiments are described herein with respect to the pelvic region to treat and/or manage pelvic conditions, they may also be used in other regions of the body or to treat other conditions as described elsewhere herein.

As a subject with an implanted electrode moves throughout their day, the implanted electrode lead may ingress (migrates deeper in the tissues of the subject) or egress (migrates to be more superficial in the tissues of the subject). An electrode lead displaced from the target implanted region may lose its utility to provide its beneficial effect of providing treatment and/or managing the condition. Thus, several embodiments described herein provide anchoring and/or fixing the electrode lead relative to a target tissue (for example an area within a pelvic region or other area of a subject) to improve the robustness and efficacy of electrical stimulation to treat and/or manage a subject's condition as a subject moves their body through the course of a day.

The devices, systems, methods, and/or kits described elsewhere herein, in some embodiments, describe an electrode lead device with one or more anchors and method of implanting the same to fix the position of the electrode lead at a target implanted location. In some embodiments, the electrode lead is anchored and/or fixed to a tissue in the target implanted location to allow for the electrode lead to move with the tissue, while still reducing migration relative to the tissue. In some embodiments, the capability of the electrode lead to move with the tissue to which it is anchored and/or fixed allows the electrode lead to keep a relatively constant distant to the target nerve even with the movement of the tissue. By fixing the position of the electrode lead relative to the tissue, the devices, systems, methods, and/or kits described elsewhere herein improve the reliability and robustness of the electrical stimulation provided over a period of time to treat and/or manage the patient's pelvic condition as a subject moves or flexes muscles adjacent the implanted electrode lead.

In several embodiments, the electrode lead bodies and surgical methods described herein have at least one or more of the following features or advantages
resist forces in the axial direction to prevent both ingress and egress;
provide stability in at least three directions, including egress, ingress, and laterally;
simplified introduction of the electrode lead body;
provide confirmation that stimulating electrodes are at target site; prior to anchor deployment;
manufacturing flexibility for different indications or patient types;
standardized implantation procedure across patients with different anatomies;
improved surgical access to the pudendal nerve and techniques for confirming access to the target site;
eliminating the use of a guidewire to maintain a determined position at the target site.

The electrode lead body may include one or more anchors to resist forces in the axial direction and prevent migration, both ingress and egress. For example, the anchors may be bi-directional. In other words, a free end of a first barb may expand toward a distal end of the lead, while a free end of a second barb may expand towards a proximal end of the lead. The first and second barbs may be on the same or different anchor. In some configurations, the anchors may provide stability in at least three directions to prevent ingress, egress, and lateralization. For example, adjacent anchors may be rotationally offset.

The methods and devices described herein simplify introduction of the electrode lead body. The anchors may be compressed for delivery to the target site and expanded once proper positioning has been confirmed. For example, the anchors may be positioned proximal of the electrodes allowing the electrodes to be deployed to confirm proper positioning, while the anchors remain collapsed within the sheath. The anchors may be semi-rigid allowing the anchors to be collapsed or compressed for delivery. With the bi-directional anchors, the anchors may all collapse in the same direction for delivery through the introducer. Certain methods described herein may utilize a lead positioning guide. The lead positioning guide is capable of positioning the electrode lead body, while the introducer constrains the anchors until the position of the electrode lead body can be verified.

The anchors may be separately attached to the electrode lead body to provide flexibility in the number and/or orientation of anchors to accommodate for different indications, target sites, or patient sizes. For example, the anchors may be separately attached to the electrode lead body using collars. This allows different numbers of anchors to be provided on the electrode lead body without changing the overall manufacturing technique.

The electrode lead body may be sufficiently flexible for introduction to the target site and correct placement along the nerve. For example, the lead body may have different flexibilities along the length of the lead body using one or more different materials or densities of the same material along the lead body. A distal portion of the lead may be stiffer than a proximal portion of the lead. For example, the tip of the lead may be stiffer than an anchor portion of the lead.

With respect to the pelvic region, there may also be challenges with accessing and confirming the location of target nerve, particularly the pudendal nerve. The pudendal nerve runs in a broadly caudad course on the inner side of the ischial bone and then turns forward to the ischiorectal fossa. The methods and instruments described herein may have at least one of the following features or advantages. For example, the electrode lead body may be introduced to the correct location with the use of a marking needle. The marking needle may indicate the point of intersection between a horizontal axis connecting both great trochanters and a vertical axis medial the ischial spine. The lines may be identified using X-ray. Moreover, pudendal nerve stimulation advantageously may provide a response in both the external anal sphincter and pelvic floor, allowing the position of the electrode lead body to be verified using an EMG response of the external anal sphincter, external urethral sphincter, and/or pelvic floor. The location of the electrode lead body may be verified using EMG in combination with one or more measurements or modalities, including urethral pressure measurements, X-ray, visual motor responses, and/or ultrasound.

Although certain devices, systems, methods, and kits are described herein with respect to the pelvic region to treat and/or manage pelvic conditions, the methods and devices can be used in other regions of the body or to treat other conditions. The anchors may be used to fix implantable devices in other regions of the body and/or to treat other conditions. In some embodiments, the anchors may be applied to electrode lead bodies implanted in the spinal region, for example to treat chronic or incidental pain. The anchors may be applied to electrode lead bodies in the spinal cord region caudal of the occiput. In other embodiments, the anchors may be applied to electrode lead bodies implanted near peripheral or cranial nerves. For example, the anchors may be applied to an electrode lead body implanted near the vagus nerve for treatment of migraines. In some embodiments, the anchors may be applied to implantable devices used outside of the field of neuromodulation, for example joint repair (e.g., shoulder, knee, or hip). The anchors may be applied to sutures used in soft tissue repair (e.g., rotator cuff repair) in place of clips. In some embodiments, the anchors may be applied to non-implantable or acute devices such as catheters, for example drug delivery catheters or drainage catheters.

Certain aspects of the disclosure relate to an electrode lead device. The electrode lead device may include a lead comprising one or more stimulation electrodes (e.g., two, three, or four or more) for applying stimulation to tissue. The electrode lead device may include a lead comprising a paddle of two or more stimulation electrodes arranged in two or more rows. The one or more stimulation electrodes may be located near a distal end of the lead. The electrode lead device may include one or more anchors (e.g., two, three, four, five, six, or more). The one or more anchors may be positioned proximal of the one or more stimulation electrodes. In some embodiments, one or more stimulation electrodes may be positioned between sets of one or more anchors. In some embodiments, one or more anchors may be positioned between sets of one or more stimulation electrodes. Each of the one or more of anchors may include a collar and one or more of barbs (e.g., two, three, four, or more) extending from the collar. The plurality of barbs may extend from the end of the collar. The plurality of barbs may extend from an end of the collar at an oblique angle relative to a longitudinal axis of the lead. The plurality of barbs may be collapsible radially inward for delivery.

The plurality of anchors may be arranged bidirectionally on the lead. The plurality of anchors may include a first set of anchors and a second set of anchors. Each barb on the first set of anchors may extend in a first direction away from the one or more stimulation electrodes. Each barb on the second set of anchors may extend in a second direction towards the one or more stimulation electrodes. The bidirectional arrangement may reduce axial migration of the lead both proximally (egress) and distally (ingress). The first set of anchors may be positioned between the one or more stimulation electrodes and the second set of anchors. The first set of anchors and the second set of anchors have a different number of anchors.

The plurality of anchors may include at least a first anchor and a second anchor that are adjacent to each other. The plurality of barbs of the first anchor may be arranged circumferentially offset from the plurality of barbs of the second anchor. The circumferential offset arrangement may reduce lateral and/or rotational migration of the lead. The plurality of barbs of the first anchor may be circumferentially offset from the plurality of barbs of the second anchor by 15 degrees, 30 degrees, 60 degrees, 90 degrees, or other values therebetween. The plurality of barbs of the first anchor may extend in a same axial direction as the plurality of barbs of the second anchor.

The electrode lead devices described herein may be configured to be implanted near a pudendal nerve. The electrode lead device may be configured to treat condition in a pelvic region comprising urinary incontinence, overactive bladder, fecal incontinence, sexual dysfunction, pelvic pain, or any combination thereof. The electrode lead device may be implanted in spinal, shoulder, knee, hip or cranial tissue. The electrode lead device may be implanted at any location used for peripheral nerve stimulation.

Certain aspects of the disclosure may include a method of affixing the electrode lead device by advancing the electrode lead device through an introducer to at a target tissue; partially withdrawing the introducer to expose the one or more stimulation electrodes; confirming a location of the one or more stimulation electrodes; and further withdrawing the introducer to expose the plurality of anchors. The method may include advancing a lead positioning guide through the introducer, the lead positioning guide carrying the electrode lead device. Confirming the location of the location of the one or more stimulation electrodes may include measuring an EMG response in an external anal sphincter. Confirming the location of the location of the one or more stimulation electrodes may include measuring an EMG response in a pelvic floor. In some implementations, robotic arm(s) and/or robotic control may be used for the surgical procedure. In some cases, stereotactic guidance may be used for manually and/or robotically-assisted placement and/or manipulation of the instruments and/or implants during the procedure.

Certain aspects of the disclosure relate to a method of implanting an electrode lead device near a pudendal nerve of a patient. The method may include advancing a stimulating member (e.g., stimulating needle or other elongate structure) toward the pudendal nerve; providing current to the stimulating member; measuring an EMG response to confirm the stimulating member is at a location near the pudendal nerve; and/or after confirming the location of the stimulating member, implanting the electrode lead device at the confirmed location. The EMG response may be measured at an external anal sphincter, external urethral sphincter, and/or pelvic floor of the patient. When no EMG responses is detected, the method may include adjusting a position of the stimulating member. Confirming the stimulating member is at the location near the pudendal nerve may include detecting a first EMG response at a pelvic floor of the patient; and detecting a second EMG response at an external anal sphincter of the patient. The method may include detecting no response between the first EMG response and the second EMG response. Confirming the stimulating member is at the location near the pudendal nerve may include detecting an EMG response in a pelvic floor of the patient before and after adjusting the position of the stimulating member. The method may include advancing a guidewire through the stimulating member, advancing an introducer over the guidewire, and/or advancing the electrode lead device through the introducer. The method may include detecting a urethral pressure to confirm the location of the stimulating member. The method may include bilaterally implanting electrode lead devices near the pudendal nerve on left and right sides of the patient.

Certain aspects of the disclosure are related to a method of implanting an electrode lead device near a pudendal nerve of a patient. The method may include inserting a marking needle, which in some instances, may also be a stimulating needle. The needle may be inserted at or near an intersection between a first line corresponding to a rim of an ischial bone (inner or outer rim) and a second line across a top of a greater trochanter. The method may include advancing a marking needle into the patient. The needle may be inserted perpendicular or at an oblique angle relative to the skin. The method may include inserting a stimulating member (e.g., stimulating needle or other elongate structure) using an ischiorectal approach toward a tip of the marking needle. The method may include confirming the stimulating member is at a location near the pudendal nerve and implanting the electrode lead device at the confirmed location. The method may include drawing the first line and the second line on a skin of the patient. Advancing the stimulating member may include advancing the stimulating member horizontally toward the tip of the marking needle. The method may include advancing a guidewire through the stimulating member, advancing an introducer over the guidewire, and/or advancing the electrode lead device through the introducer to the confirmed location. Confirming the stimulating member is at a location near the pudendal nerve may include providing a current to the stimulator member of less than or equal to 3 mA. Confirming the location of the stimulating member comprises measuring an EMG response of at least 20 μV.

Certain aspects of the disclosure relate to a method of implanting an electrode lead device as described herein. The method may include introducing the electrode lead device into a lead positioning guide until the one or more stimulation electrodes extend beyond a distal end of the lead positioning guide; advancing the lead positioning guide through an introducer until the one or more stimulation electrodes are positioned at a distal portion of the introducer; partially withdrawing the introducer to expose the one or more stimulation electrodes while the plurality of anchors remain constrained within the introducer; measuring an EMG response to confirm a location of the one or more stimulation electrodes; adjusting a position of the one or more stimulation electrodes until a desired EMG response is measured; and/or further withdrawing the introducer to release the plurality of anchors. The method may include axially adjusting a position of the one or more stimulation electrodes within a patient without releasing the plurality of anchors from the introducer. The method may include partially withdrawing the introducer until some of the electrodes are exposed and some are sheathed, e.g., two of four electrodes are exposed and the remaining two are sheathed. The method may include partially withdrawing the introducer until a handle of the introducer contacts arms on the lead positioning guide. The method may include rotating the lead positioning guide relative to the introducer to allow the further withdrawal of the introducer. The method may include partially withdrawing the introducer until a handle of the introducer is coupled with the lead positioning guide. The method may include decoupling the lead positioning guide from the introducer to allow the lead positioning guide to release the plurality of anchors.

In several embodiments, an electrode lead device may have a lead body having one or more electrodes and one or more anchors (for example two to eight electrodes, some or all of which may be stimulating, and two to eight anchors). The lead body may have the same or varying materials (e.g., different densities) along the lead body to provide varying levels of flexibility along the lead body. The electrodes may be located near a distal end of the lead. One or more (or all) of the anchors may be positioned proximal of the one or more stimulation electrodes configured to contact or apply stimulation to the tissue. Each anchor may include an anchor body portion (such as a “collar”) having one or more barbs (e.g., 1-6 barbs) extending, directly or indirectly, from the collar. For example, each anchor may have two barbs diametrically opposed from each other. The collars may be separately attached to the lead body enabling different numbers of anchors to be secured to the lead body depending on the procedure. The barbs may be collapsed or otherwise be sufficiently flexible/malleable for delivery. The anchors may be arranged bi-directionally with a first set of anchors having barbs extending distally (e.g., toward the stimulation electrodes) and a second set of anchors having barbs extending proximally (e.g., away from the stimulation electrodes). The second set of anchors may be grouped between the first set of anchors and the stimulation electrodes. The first and second sets of anchors may have the same or different number of anchors. At least one anchor may be circumferentially offset from another anchor, for example by 80 degrees to 100 degrees, such as 85 degrees, 90 degrees, 100 degrees, or other values within the range. In some embodiments, adjacent anchors may be circumferentially offset from each other. Optionally, each barb may extend from an end of its respective collar at an oblique angle relative to a longitudinal axis of the lead body (e.g., less than 45 degrees, less than 30 degrees, or less than 20 degrees). Each barb may have a free end having an edge that is rounded or generally flat (e.g., perpendicular to a longitudinal axis of the lead body). A length of the edge may be at least half a diameter of the lead body. The free end of the barb may be tapered or chamfered edge in the radial direction. The electrode lead body may include one or more stoppers at either or both ends of the array of anchors or therebetween. The stopper(s) may ensure the anchors do not migrate or slip off the lead during revision or other high axial forces. The stopper may be a tubular body, for example made of pelethane. The tubular body may be longer than one of the anchors.

Methods of providing access to the pudendal nerve are described herein. The method may include drawing a first line corresponding to a rim of an ischial bone (inner room or outer rim) and drawing a second line across a top of a greater trochanter. The method may include inserting a marking needle at the intersection between the first line and the second line and advancing the marking needle until the marking needle comes into contact with an ischial spine of the ischial bone. The first line and the second line may be identified based on X-ray. In some embodiments, the marking needle is a stimulating needle. The stimulating needle may be inserted until there is EMG activity at the external anal sphincter in response to a stimulation at less than or equal to 3 mA or less than or equal to 2 mA. The EMG response may be indicative of the tip of the stimulating needle being positioned at or near the pudendal nerve. The method may include inserting a stimulating member (e.g., stimulating needle or other elongate structure) using an ischiorectal approach and toward the marking needle or stimulating needle. A position of the stimulating member may be confirmed using EMG. For example, an EMG response in the pelvic floor followed by an EMG response at the external anal sphincter may indicate optimal positioning of the stimulating member. If a response is not detected, the stimulating member may be adjusted. In some methodologies, the positioning may be confirmed following detection of only an EMG response in the pelvic floor following each adjustment. After the position of the stimulating member is confirmed, any of the electrode lead devices described herein may be implanted at the location of the stimulating member. In some methodologies, a position of the stimulating member may be confirmed based only on an EMG response at the external anal sphincter.

Electrode lead devices described herein may be delivered using a lead positioning guide, which allows for confirmation of the stimulation electrodes at the target site prior to deploying the anchors. The lead is inserted into the lead positioning guide until there is contact between a distal end of the lead positioning guide and the proximal-most anchor on the lead. After the lead is locked within the lead positioning guide, the lead positioning guide is inserted into the introducer until the stimulation electrodes are positioned at a distal portion of the introducer. For example, the lead may be locked within the lead positioning guide using a locking nut, which may be a tapered nut. The lead positioning guide may have indicators (e.g., marker bands) to provide an indication of when the stimulation electrodes are positioned at the distal portion of the introducer. The introducer may be withdrawn to expose the stimulation electrodes while the anchors remain constrained within the introducer. The lead positioning guide and/or the introducer may have alignment features to indicate when only the stimulation electrodes have been exposed from the distal end of the introducer. In this configuration, the location of the stimulation electrodes may be adjusted until the clinician has confirmed the stimulation electrodes are in the correct position. The lead positioning guide and/or the introducer may have features to prevent further withdrawal of the introducer until the position of the stimulation electrodes has been confirmed. After the location of the stimulation electrodes has been confirmed, the lead positioning guide may be rotated relative to the introducer to permit further withdrawal of the introducer. The introducer may be further withdrawn to deploy the anchors.

Aspects of the disclosure describe an electrode lead device for treating a condition, for example in the pelvic region, the device may include one or more of the following features: a lead comprising one or more stimulation electrodes located near a distal end of the lead; and a plurality of anchors, each anchor comprising a collar and two or more barbs extending from the collar, where the plurality of anchors is positioned on the lead proximal to the one or more stimulation electrodes, where a first anchor of the plurality of anchors is adjacent to a second anchor of the plurality of anchors, where the two or more barbs of the first anchor are positioned at a rotation angle from two or more barbs of the second anchor along a length of the lead, and/or where the one or more stimulation electrodes is configured to deliver electrical stimulation to a target tissue. In some embodiments, the target tissue comprises a pudendal nerve or a tissue adjacent the pudendal nerve. In some embodiments, the condition is in the pelvic region and comprises urinary incontinence, fecal incontinence, sexual dysfunction, voiding dysfunction, pelvic pain, or any combination thereof. In some embodiments, the first anchor or the second anchor are releasably coupled to the lead. In some embodiments, the two or more barbs comprises 2, 3, 4, 5, 6, 7, 8, 9, or 10 barbs. In some embodiments, the two or more barbs are equally spaced apart around the collar. In some embodiments, the two or more barbs are radially symmetric around the collar. In some embodiments, the two or more barbs are not radially symmetric around the collar. In some embodiments, a first barb of the two or more barbs of the first anchor or second anchor is positioned at a rotation angle (for example about 1 degree to about 180 degrees, between about 0 degrees to about 30 degrees, between about 15 degrees to about 45 degrees, between about 30 degrees to about 60 degrees, between about 45 degrees to about 75 degrees, between about 60 degrees to about 90 degrees, between about 75 degrees to about 105 degrees, between about 90 degrees to about 120 degrees, between about 105 degrees to about 135 degrees, between about 120 degrees to about 150 degrees, between about 135 degrees to about 165 degrees, between about 150 degrees to about 180 degrees) from a second barb of the two or more barbs of the first anchor or the second anchor. In some embodiments, each of the two or more barbs are configured to extend along a radius of a circular cross-section of the collar. In some embodiments, the plurality of anchors comprises a third anchor, wherein the third anchor is adjacent to the second anchor or the first anchor. In some embodiments, the third anchor comprises a first barb of two more barbs positioned at a rotation angle from a second barb of the two more barbs of the first anchor or the second anchor. In some embodiments, the first anchor and second anchor have a gap in between the first anchor and the second anchor. In some embodiments, the gap comprises a length of about 1 mm to about 5 mm. In some embodiments, the gap is between a surface of a free end of a barb of the two more barbs of the first anchor to a surface of the collar of the second anchor. In some embodiments, the two or more barbs are configured to extend away at an angle with respect to an axial axis of the collar. In some embodiments, the collar comprises an inner diameter of at least about 1.3 mm. In some embodiments, the collar comprises a thickness of at least about 0.35 mm. In some embodiments, the length of the collar comprises a length of at least about 2.5 mm. In some embodiments, the length of the collar comprises a length of up to about 5 mm. In some embodiments, the length of the collar comprises a length of about 2 mm to about 5 mm. In some embodiments, the outer diameter of the collar is at least about 2 mm. In some embodiments, the two or more barbs comprise a length of at least about 1.5 mm. In some embodiments, when the two or more barbs are extended, the two or more barbs form a radius of at least about 1.25 mm between an outer surface of the two or more barbs and an outer surface of the collar. In some embodiments, when the two or more barbs extend along a radial axis of a cross section of the collar at an angle of about 10 degrees to about 80 degrees from an axial axis of the collar (for example, less than or equal to 60 degrees, less than or equal to 50 degrees, less than or equal to 45 degrees, less than or equal to 40 degrees, or less than or equal to 30 degrees). In some embodiments, a free end of a barb of the two or more barbs of the first anchor expand toward a distal end of the lead, and wherein a free end of a barb of the two or more barbs of the second anchor expand towards a proximal end of the lead. In some embodiments, the two or more barbs comprise thermally set barbs, and wherein the thermally set barbs in an expanded state expand to an angle of about 20 degrees to about 65 from an axial axis of the collar. In some embodiments, the two or more barbs comprise a width of at least about 0.2 mm. In some embodiments, the two or more barbs comprise a thickness of at least about 0.2 mm. In some embodiments, the two or more barbs comprise a thickness of up to about 0.35 mm. In some embodiments, the two or more barbs comprise a thickness of about 0.2 mm to about 0.35 mm. In some embodiments, the two or more barbs comprise a rectangular or triangular profile. In some embodiments, the two or more barbs comprise a profile that matches a curvature of a surface of the collar. In some embodiments, one or more edges of a free end of the pair of (tines or barbs) are chamfered or filleted edges. In some embodiments, the one or more fileted edges of a free end of the two or more barbs comprise a radius of at least about 0.35 mm. In some embodiments, the first anchor or the second anchor are made from a polymer. In some embodiments, the polymer comprises thermoplastic polyurethane elastomer (TPU). In some embodiments, the polymer comprises polytetrafluoroethylene (PTFE). In some embodiments, the first anchor and the second anchor are made from a material with a stiffness of at least about Shore 55 D. In some embodiments, the first anchor and the second anchor are made from a material with a stiffness of at most about 75 D. In some embodiments, the first anchor and the second anchor are made from a material with a stiffness of about Shore 55 D to about Shore 75 D. In some embodiments, the first anchor and the second anchor comprise a serrated profile. In some embodiments, the serrated profile comprises one or more cut features disposed along an edge of a barb of the two or more barbs. In some embodiments, the one or more cut features comprise a circular geometry, wherein the circular geometry comprises a diameter of about 0.25 mm to about 0.5 mm. In some embodiments, the device further comprises a sheath covering at least a portion of first anchor or the second anchor. In some embodiments, the sheath comprises a hardness of at least about 60 D, 65 D, 70 D, 75 D, 80 D, 85 D, or 90 D shore hardness. In some embodiments, the device further comprising an introducer, where the introducer comprises a lumen configured to receive the lead and the first anchor or the second anchor and guide implantation of the lead and the first anchor or the second anchor. In some embodiments, the introducer is configured to collapse or compact the first anchor or the second anchor as the lead is advance towards a distal end of the introducer. In some embodiments, the introducer is made from a polymer material or metal. In some embodiments, the metal comprises stainless steel, aluminum, titanium, or any combination thereof. In some embodiments, the device further comprises a lead positioning guide (LPG), wherein the LPG comprises a lumen diameter configured to receive the lead, and wherein the LPG fixes a position of the lead as the introducer is retracted over the lead and the first anchor or the second anchor. In some embodiments, the introducer comprises a lumen diameter configured to receive the LPG. In some embodiments, the polymer comprises thermoplastic polyurethane. In some embodiments, the polymer comprises polytetrafluoroethylene (PTFE). In some embodiments, the introducer comprises a length of the about 150 mm to about 400 mm. In some embodiments, the introducer comprises a conical or tapered cross-sectional profile. In some embodiments, the conical or the tapered cross-sectional profile of the introducer and a rounded edge of a free end of the two or more barbs reduce friction between the rounded edge of the free end of the two or more barbs and an inner lumen of the introducer. In some embodiments, the inner lumen of the introducer comprises a first inner diameter and a second inner diameter, wherein the first inner diameter and the second inner diameter are different. In some embodiments, the inner lumen of the introducer has an inner diameter ranging from about 0.5 mm to about 5 mm (e.g., 0.5-1, 1-2, 2-3, 3-4, 4-5mm and overlapping ranges therein). In some embodiments, the first inner diameter is smaller than the second inner diameter. In some embodiments, the introducer is retracted over the first anchor or the second anchor coupled to the lead thereby expanding the two or more barbs of the first anchor or the second anchor to fix the lead at the tissue at or near the pudendal nerve. In some embodiments, the introducer comprises a size of about 1 French (F) to about 15 F, for example less than or equal to 10 F (3 F, 4 F, 5 F, 6 F, 7 F, or other values). In some embodiments, the first anchor or the second anchor are implanted in connective tissue. In some embodiments, the connective tissue comprises a sacrotuberous ligament, sacrospinous ligament, fascial and periosteal tissues of the falciform process or a combination thereof. In some embodiments, the first anchor or the second anchor are manufactured by additive 3D printing, laser cutting, injection molding or a combination thereof. In some embodiments, the additive 3D printing comprises selective laser sintering.

Aspects of the disclosure describe a method of affixing an electrode lead to a tissue, the method may include one or more of the following features or steps: placing an electrode lead at a target tissue, wherein the electrode lead comprises: (i) a lead comprising one or more stimulation electrodes located near a distal end of the lead; and (ii) a plurality of anchors, each anchor comprising a collar and two or more barbs extending from the collar, where the plurality of anchors is releasably positioned on the lead proximal to the one or more stimulation electrodes, where a first anchor of the plurality of anchors is adjacent to a second anchor of the plurality of anchors, where the two or more barbs of the first anchor are positioned at a rotation angle from two or more barbs of the second anchor along a length of the lead, and where the one or more stimulation electrodes is configured to deliver electrical stimulation to a target tissue; and removing a sheath covering at least a portion of the first anchor or the second anchor to deploy the first anchor or the second anchor to anchor the electrode lead to the target tissue. In some embodiments, the target tissue comprises a pudendal nerve or tissue adjacent the pudendal nerve. In some embodiments, the target tissue comprises a target tissue in a pelvic region. In some embodiments, the method further comprises stimulating the target tissue with the electrode lead to treat a condition a pelvic region. In some embodiments, the condition comprises urinary incontinence, fecal incontinence, pain, sexual dysfunction, or any combination thereof. In some embodiments, the first anchor or the second anchor are releasably coupled to the electrode lead. In some embodiments, the two or more barbs comprises 2, 3, 4, 5, 6, 7, 8, 9, or 10 barbs. In some embodiments, the two or more barbs are equally spaced apart around the collar. In some embodiments, the two or more barbs are radially symmetric around the collar. In some embodiments, the two or more barbs are not radially symmetric around the collar. In some embodiments, a first barb of the two or more barbs of the first anchor or second anchor is positioned at a rotation angle of about 1 degree to about 180 degrees from a second barb of the two or more barbs of the first anchor or the second anchor. In some embodiments, each of the two or more barbs are configured to expand along a radius of a circular cross-section of the collar. In some embodiments, the plurality of anchors comprises a third anchor, wherein the third anchor is adjacent to the second anchor or the first anchor. In some embodiments, the third anchor comprises a first barb of two or more barbs positioned at a rotation angle from a second barb of the two more barbs of the first anchor or the second anchor. In some embodiments, the first anchor and second anchor have a gap in between the first anchor and the second anchor. In some embodiments, the gap comprises a length of about 1 mm to about 5 mm. In some embodiments, the gap is between a surface of a free end of a barb of the two more barbs of the first anchor to a surface of the collar of the second anchor. In some embodiments, the two or more barbs are configured to extend away at an angle with respect to an axial axis of the collar. In some embodiments, the collar comprises an inner diameter of at least about 1.30 mm. In some embodiments, the collar comprises a thickness of at least about 0.35 mm. In some embodiments, the length of the collar comprises a length of at least about 2.5 mm. In some embodiments, the length of the collar comprises a length of up to about 5 mm. In some embodiments, the length of the collar comprises a length of about 2 mm to about 5 mm. In some embodiments, the outer diameter of the collar is at least about 1 mm. In some embodiments, the two or more barbs comprise a length of at least about 1.5 mm. In some embodiments, the when the two or more barbs are extended, the two or more barbs form a radius of at least about 1.25 mm between an outer surface of the two or more barbs and an outer surface of the collar. In some embodiments, when the two or more barbs extend along a radial axis of a cross section of the collar at an angle of about 10 degrees to about 80 degrees from an axial axis of the collar. In some embodiments, a free end of a barb of the two or more barbs of the first anchor expand towards a distal end of the electrode lead, and where a free end of a barb of the two or more barbs of the second anchor expand towards a proximal end of the electrode lead. In some embodiments, the two or more barbs comprise thermally set barbs, and wherein the thermally set barbs in an expanded state expand to an angle of about 20 degrees to about 65 from an axial axis of the collar. In some embodiments, the two or more barbs comprise a width of at least about 0.2 mm. In some embodiments, the two or more barbs comprise a thickness of at least about 0.2 mm. In some embodiments, the two or more barbs comprise a thickness of up to about 0.35 mm. In some embodiments, the two or more barbs comprise a thickness of about 0.2 mm to about 0.35 mm. In some embodiments, the two or more barbs comprise a rectangular or triangular profile. In some embodiments, the two or more barbs comprise a profile that matches a curvature of a surface of the collar. In some embodiments, one or more edges of a free end of the pair of (tines or barbs) are chamfered or filleted edges. In some embodiments, the one or more fileted edges of a free end of the two or more barbs comprise a radius of at least about 0.35 mm. In some embodiments, the first anchor or the second anchor are made from a polymer. In some embodiments, the polymer comprises thermoplastic polyurethane elastomer (TPU). In some embodiments, the polymer comprises polytetrafluoroethylene (PTFE). In some embodiments, the first anchor and the second anchor are made from a material with a stiffness of at least about Shore 55 D. In some embodiments, the first anchor and the second anchor are made from a material with a stiffness of at most about 75 D. In some embodiments, the first anchor and the second anchor are made from a material with a stiffness of about Shore 55 D to about Shore 75 D. In some embodiments, the first anchor and the second anchor comprise a serrated profile. In some embodiments, the serrated profile comprises one or more cut features disposed along an edge of a barb of the two or more barbs. In some embodiments, the one or more cut features comprise a circular geometry, wherein the circular geometry comprises a diameter of about 0.25 mm to about 0.5 mm. In some embodiments, the sheath comprises a hardness of at least about 60 D, 65 D, 70 D, 75 D, 80 D, 85 D, or 90 D shore hardness. In some embodiments, the sheath is made from an aromatic polyether-based thermoplastic polyurethane. In some embodiments, the method further comprises collapsing or compacting the two or more barbs of the first anchor or the second anchor when the first anchor or the second anchor are translated axially through an introducer. In some embodiments, the introducer is made from a polymer material or a metal. In some embodiments, the metal comprises stainless steel, aluminum, titanium, or any combination thereof. In some embodiments, the polymer comprises thermoplastic polyurethane. In some embodiments, the polymer comprises polytetrafluoroethylene (PTFE). In some embodiments, the introducer comprises a length of the about 150 mm to about 400 mm. In some embodiments, the introducer comprises a conical or tapered cross-sectional profile. In some embodiments, the conical or the tapered cross-sectional profile of the introducer and a rounded edge of a free end of the two or more barbs reduce friction between the rounded edge of the free end of the two or more barbs and an inner lumen of the introducer. In some embodiments, the inner lumen of the introducer comprises a first inner diameter and a second inner diameter, wherein the first inner diameter and the second inner diameter are different. In some embodiments, the first inner diameter comprises a diameter of at least about 3.8 mm, and wherein the second inner diameter comprises a diameter of at least about 6.5 mm. In some embodiments, the introducer is retracted over the first anchor or the second anchor coupled to the electrode lead thereby expanding the two or more barbs of the first anchor or the second anchor to fix the electrode lead at the tissue at or near the pudendal nerve. In some embodiments, the introducer comprises a size of about 1 French (F) to about 15 F. In some embodiments, the method further comprises fixing a position of the electrode lead with a lead positioning guide (LPG) when the sheath covering at least the portion of the first anchor or the second anchor is removed. In some embodiments, the introducer comprises a lumen configured to receive the LPG. In some embodiments, the first anchor or the second anchor are implanted in connective tissue. In some embodiments, the connective tissue comprises a sacrotuberous ligament, sacrospinous ligament, other dense tissues in the region medial to the ischial tuberosity, or a combination thereof. In some embodiments, the first anchor or the second anchor are manufactured by additive 3D printing, laser cutting, injection molding or a combination thereof. In some embodiments, the additive 3D printing comprises selective laser sintering.

Aspects of the disclosure describe a kit for treating a condition, for example in a pelvic region, the kit may include one or more of the following components: (a) an electrode lead device, comprising: (i) a lead comprising one or more stimulation electrodes located near a distal end of the lead; and (ii) a plurality of anchors, each anchor comprising a collar and two or more barb extending from the collar, where the plurality of anchors is provided at a fixed positioned on the lead proximal to the one or more stimulation electrodes, where a first anchor of the plurality of anchors is adjacent to a second anchor of the plurality of anchors, where the two or more barbs of the first anchor are positioned at a rotation angle from two or more barbs of the second anchor along a length of the lead, and where the one or more stimulation electrodes is configured to deliver electrical stimulation to a target tissue; and (b) instructions for placing or anchoring the electrode lead to the target tissue. In some embodiments, the target tissue comprises a pudendal nerve or a tissue adjacent the pudendal nerve. In some embodiments, the condition is in the pelvic region and comprises urinary incontinence, fecal incontinence, pain, sexual dysfunction, or any combination thereof. In some embodiments, the instructions comprise instructions in an insert, on a website, or a combination thereof. In some embodiments, the two or more barbs comprises 2, 3, 4, 5, 6, 7, 8, 9, or 10 barbs. In some embodiments, the two or more barbs are equally spaced apart around the collar. In some embodiments, the two or more barbs are radially symmetric around the collar. In some embodiments, the two or more barbs are not radially symmetric around the collar.

Aspects of the disclosure describe a method of manufacturing an anchor, the method may include one or more of the following features or steps: (a) molding a first half and a second half of an anchor body, where the first half of the anchor comprises a first barb region, and the second half of the anchor body comprises a second barb region, where the first half of the anchor body is made of the same material as the first barb region, and where the second half of the anchor is made of the same material of the second barb region; and (b) fixing the first half and the second half of the anchor body to form the anchor body. In some embodiments, molding comprising injection molding. In some embodiments, the first half of the anchor body and the second half of the anchor body comprise a removably coupled feature configured to be (removed or broken off) the first half or the second half of the anchor body. In some embodiments, the first half or the second half of the anchor body is molded from a polymer. In some embodiments, the polymer comprises thermoplastic polyurethane elastomer (TPU). In some embodiments, the polymer comprises thermoplastic polyurethane. In some embodiments, the polymer comprises polytetrafluoroethylene (PTFE). In some embodiments, the first half or the second half of anchor body is molded from a material with a stiffness of at least about Shore 55 D.

In some aspects, the techniques described herein relate to a system for placing an electrode in pelvic region, the system including one or more of the following: a lead including one or more stimulation electrodes located at or near a distal end of the lead; and a plurality of anchors, each anchor including a collar and two or more barb extending from the collar, wherein the plurality of anchors is positioned on the lead proximal to the one or more stimulation electrodes; an introducer configured to be slidable over the lead and the plurality of anchors, the introducer having an introducer handle; a lead positioning guide including an elongated guide body having a collet at a proximal end of the elongated guide body, a guide lumen through the elongated guide body and one or more guide arms extending distally from the elongate guide body, and a locking cap configured to fit over the collet, wherein the one or guide arms is configured to engage with the introducer handle. In some aspects, the techniques described herein relate to a system, wherein the one or more guide arms is two guide arms. In some aspects, the techniques described herein relate to a system, wherein the introducer handle has a proximal surface including one or more protrusions including a stem and an overhang over the stem. In some aspects, the techniques described herein relate to a system, wherein the one or guide arms has a distal end including an arm stem and an arm overhang. In some aspects, the techniques described herein relate to a system, wherein a thickness of the arm overhang is substantially similar to or less than a height of the stem of the introducer handle. In some aspects, the techniques described herein relate to a system, wherein the arm overhang is configured to removably fit under the overhang of the introducer handle by the stem. In some aspects, the techniques described herein relate to a system, wherein the arm overhang is configured to removably fit over the proximal surface of the introducer handle. In some aspects, the techniques described herein relate to a system, wherein when the arm overhang is under the overhang of the introducer handle by the stem, the introducer handle is securely fixed to the lead positioning guide. In some aspects, the techniques described herein relate to a system, wherein the introducer is securely fixed in the proximal-distal direction relative to the lead positioning guide. In some aspects, the techniques described herein relate to a system, wherein a rotation of the lead positioning guide about the proximal-distal axis relative to the introducer handle changes a level of engagement of the one or more guide arms with the introducer handle. In some aspects, the techniques described herein relate to a system, wherein the rotation of the lead positioning guide disengages the one or more guide arms from the introducer handle. In some aspects, the techniques described herein relate to a system, wherein the introducer is moveable in the proximal-distal direction relative to the lead positioning guide. In some aspects, the techniques described herein relate to a system, wherein the arm overhang is at an angle (e.g., an L-shape) from the arm step. In some aspects, the techniques described herein relate to a system, wherein the arm overhang includes a curved face, a hook-shaped face, a rectangular face, or a combination thereof. In some aspects, the techniques described herein relate to a system, wherein the overhang of the introducer handle engages with the arm overhang. In some aspects, the techniques described herein relate to a system, wherein the guide lumen is configured to slideably fit the lead. In some aspects, the techniques described herein relate to a system, wherein the collet has a proximal end that is tapered. In some aspects, the techniques described herein relate to a system, wherein the collet has a diameter that is reduced when the locking cap moves distally over the collet from an unlocked position to a locked position. In some aspects, the techniques described herein relate to a system, wherein the guide lumen at the collet has a diameter that is reduced when the locking cap moves distally over the collet from an unlocked position to a locked position. In some aspects, the techniques described herein relate to a system, wherein when the lead is placed through the guide lumen, the lead is secured in the guide lumen of the elongated guide body by the locking cap is in the locked position over the collet. In some aspects, the techniques described herein relate to a system, wherein the lead is secured in the guide lumen of the collet in a proximal-distal direction. In some aspects, the techniques described herein relate to a system, wherein the elongated guide body has a thread. In some aspects, the techniques described herein relate to a system, wherein the locking cap has a thread configured to fit with the thread of the elongated guide body and move the locking cap in the proximal-distal direction. In some aspects, the techniques described herein relate to a system, wherein when the lead is placed through the guide lumen, the lead is secured in the guide lumen of the elongated guide body by the locking cap. In some aspects, the techniques described herein relate to a system, wherein the lead includes a wire is rotated variably along a portion of the lead. In some aspects, the techniques described herein relate to a system, wherein the wire has about 2 to about 15 turns per 70 mm of the lead. In some aspects, the techniques described herein relate to a system, wherein the wire has about 7 to about 10 turns per 70 mm of the lead. In some aspects, the techniques described herein relate to a system, wherein the wire has about 8 turns per 70 mm of the lead. In some aspects, the techniques described herein relate to a system, wherein the wire has about 1 to about 5 full rotations per 70 mm of the lead. In some aspects, the techniques described herein relate to a system, wherein the wire has about 1.5 to about 2.5 full rotations per 70 mm of the lead. In some aspects, the techniques described herein relate to a system, wherein the wire has about 2 full rotations per 70 mm of the lead. In some aspects, the techniques described herein relate to a system, wherein the wire varies by at least about 0.1, 0.5, 1, 1.5, 2, 2.5, 3, 3.5, 4, 4.5, 5, 5.5, 6, 6.5, 7, 7.5, or 8 turns per 70 mm at two different portions of the lead. In some aspects, the techniques described herein relate to a system, wherein a rotation of the wire is lower at or near a connector, a stiffener, or an electrode, or a combination thereof. In some aspects, the techniques described herein relate to a system, wherein the wire includes a sufficiently malleable material to allow for variable coiling. In some aspects, the techniques described herein relate to a system, wherein the wire includes a platinum alloy. In some aspects, the techniques described herein relate to a system, wherein the wire includes a platinum iridium. In some aspects, the techniques described herein relate to a system, wherein the elongated guide body includes at least two radiopaque markers.

In some aspects, the techniques described herein relate to a method having a plurality over anchors on the lead into a guide lumen until a proximal anchor in the plurality over the lead and a portion of the elongate guide body of the lead positioning guide to cover the one or more stimulation electrodes on the lead and the plurality of anchors; delivering the lead having the plurality over anchors and a distal portion of the introducer into a target location in the pelvic region using an introducer handle of the introducer and the lead positioning guide; sliding the introducer handle proximally to engage with one or more guide arms of the lead positioning guide to secure the introducer in a proximal-distal direction and to expose one or more stimulation electrodes on the lead while covering the plurality of anchors; verifying the delivery of the exposed one or more stimulation electrodes to the target location; moving the lead positioning guide to disengage from the introducer handle; sliding the introducer handle proximally to expose the plurality of anchors to secure the lead in place in the pelvic region; and moving the locking cap from the locked position to the unlocked position; and withdrawing the introducer and the lead positioning guide from the pelvic region. In some aspects, the techniques described herein relate to a method, wherein the method further includes adjusting a location of the one or more stimulation electrodes and repeating step (f). In some aspects, the techniques described herein relate to a method, wherein the locking cap is moved distally over a collet of the lead positioning guide in the locked position. In some aspects, the techniques described herein relate to a method, wherein the locking cap is rotated distally over a collet of the lead positioning guide in the locked position. In some aspects, the techniques described herein relate to a method, wherein the locking cap is moved proximally over a collet of the lead positioning guide in the unlocked position. In some aspects, the techniques described herein relate to a method, wherein the locking cap is rotated proximally over a collet of the lead positioning guide in the unlocked position. In some aspects, the techniques described herein relate to a method, wherein in step (h) two or more barbs of an anchor of the plurality of anchors extends outwardly to secure the lead into a surrounding tissue. In some aspects, the techniques described herein relate to a method, wherein verifying in step (f) includes using an EMG. In some aspects, the techniques described herein relate to a method, wherein the elongated guide body includes a radiopaque marker for indicating a location to place the introducer in step (c).

Certain aspects of the disclosure relate to a method of placing an implantable neuromodulation lead in a pelvic area of an individual. The method may include obtaining an x-ray image of the pelvic area of the individual. In some embodiments, the image may be another type of medical image, e.g., CT scan, MRI scan, etc., disclosed elsewhere herein. The x-ray image may be in an anterior-posterior (AP) view or a posterior-anterior (PA) view. The method may further include identifying one or more anatomical landmarks on the x-ray image, or any other medical image, of the pelvic area. The one or more anatomical landmarks may be one or more bone landmarks associated with a pelvis. In some embodiments, the bone landmarks may be associated with obturator foramina of the pelvis. In some embodiments, identifying the one or more anatomical landmarks may comprise identifying using a trained machine learning and/or artificial intelligence model. The method may further include determining, based on said one or more anatomical landmarks, an adjustment of an orientation of an operating table for positioning the individual. The method may further include adjusting the operating table based on the adjustment. In some embodiments, adjusting the operating table comprises causing an actuator to automatically adjust the operating table. The adjusting the operating table may comprise lowering a left hand side, a right hand side, a head end, and/or a foot end of the operating table. The method may further include, with the individual lying on the adjusted operating table, inserting a stimulation needle assembly into a body of the individual along a path to a target location at or near a nerve of the individual. The nerve may be a pudendal nerve or a sacral nerve. The stimulation needle assembly may comprise a needle having an elongate needle body. The method may further include delivering electrical stimulation from an external source via the stimulation needle assembly to tissue of the individual to elicit a response in the individual indicative of a distal tip of the stimulation needle assembly being at the target location. The method may further include advancing an introducer assembly along the path to the target location. The introducer assembly may comprise an introducer sheath comprising an inner lumen. The method may further comprise advancing the implantable neuromodulation lead to the target location through the inner lumen of the introducer sheath.

In some embodiments, inserting the stimulation needle assembly or advancing the introducer assembly does not include use of a guidewire. The advancing the introducer assembly may comprise advancing over the elongate needle body of the stimulation needle assembly. The method may further include withdrawing the needle from the introducer assembly. The method may further include delivering the electrical stimulation from the stimulation needle assembly as the introducer assembly is advanced over the elongate needle body and into the body of the individual to verify the distal tip of the stimulation needle assembly remains at the target location. In some embodiments, the method may further include providing the introducer assembly over the stimulation needle assembly prior to inserting the stimulation needle assembly into the body. The needle may include a hub at a proximal end of the needle. The length of the elongate needle body may be greater than a length of the introducer assembly by at least 50 mm. In some embodiments, the needle may not include an integral hub. The advancing the introducer assembly may comprise advancing a distal end of the introducer assembly over a proximal end of the needle. The stimulation needle assembly may further comprise a removable stylet comprising a stylet body and a stylet hub, wherein the stylet is configured to deliver the electrical stimulation. The stimulation needle assembly may comprise a removable hub configured to be coupled to the elongate needle body and provide an electrical connection between the needle and the external source of stimulation. The removable hub may be removed from the needle body before advancing the introducer assembly over the elongate needle body.

Certain aspects of the disclosure relate to a method of placing an implantable neuromodulation lead in a pelvic area of an individual. The method may include inserting a stimulation needle assembly into a body of the individual along a path to a target location at or near a pudendal nerve of the individual. The stimulation needle assembly may comprise a needle having an elongate needle body. In some embodiments, the needle may not comprise an inner lumen. The method may further include delivering electrical stimulation from an external source via the stimulation needle assembly to tissue of the individual to elicit a response in the individual indicative of a distal tip of the stimulation needle assembly being at the target location. The method may further include advancing an introducer assembly over the elongate needle body along the path to the target location, the introducer assembly comprising an introducer sheath comprising an inner lumen. The method may further include withdrawing the needle from the introducer assembly. The method may further include, after withdrawing the needle, advancing the implantable neuromodulation lead to the target location through the inner lumen of the introducer sheath. The inserting of the stimulation needle assembly or advancing the introducer assembly may not include use of a guidewire. The method may further comprise delivering continued electrical stimulation from the stimulation needle assembly as the introducer assembly is advanced over the elongate needle body and into the body of the individual to verify the distal tip of the stimulation needle assembly remains at the target location.

In some embodiments, the needle may not include an integral hub. The advancing the introducer assembly may comprise advancing a distal end of the introducer assembly over a proximal end of the needle. The stimulation needle assembly may comprise a removable stylet comprising a stylet hub and configured to deliver the electrical stimulation. In some embodiments, the stimulation needle assembly may comprise a removable hub configured to be coupled to the elongate needle body and provide an electrical connection between the needle and the external source of stimulation. The removable hub may be removed from the needle body before advancing the introducer assembly over the elongate needle body.

In some embodiments, the method may include providing the introducer assembly over the stimulation needle assembly prior to inserting the stimulation needle assembly into the body. The needle may or may not include an integral hub at a proximal end of the needle. The stimulation needle assembly may additionally, or alternatively, comprise a removable hub. A length of the elongate needle body may be greater than a length of the introducer assembly by at least 50 mm.

Certain aspects of the disclosure relate to a kit for implantation of a stimulation lead in a pelvic area of an individual. The kit may include a stimulation needle assembly. The stimulation needle assembly may comprise a needle comprising an elongate needle body with an outer diameter and a length. The length of the elongate needle body may be at least 150 mm. The outer diameter of the elongate needle body may be at least 20 Gauge. The needle body may include an inner lumen with a diameter less than 0.5 mm, or may not include an inner lumen. The needle may comprise a distal tip. The needle may be configured to be advanced through body tissue along a path to a target location in the pelvic area of the individual. The stimulation needle assembly may be configured to deliver electrical stimulation from an external source to elicit a response in the individual indicative of a location of a distal tip of the stimulation needle assembly. The kit may further include an introducer assembly. The introducer assembly may comprise an introducer sheath. In some embodiments, the introducer assembly may further include an obturator within the introducer sheath. An innermost lumen diameter of the introducer assembly may be sized such that the introducer assembly is advanceable over the elongate needle body along the path to the target location. A difference between the outer diameter of the elongate needle body and an innermost diameter of the introducer assembly may be less than 0.5 mm. The stimulation needle assembly may be configured to deliver continued electrical stimulation as the introducer assembly is advanced along the path. The elongate needle body may be configured to be withdrawn from the introducer assembly. In some embodiments, the obturator may be withdrawn from the introducer sheath. The introducer sheath may be configured to receive a stimulation lead. The kit may further include a removable hub configured to be coupled to the elongate needle body at least 50 mm distally away from a proximal end of the needle. In some embodiments, the introducer assembly may be provided over the stimulation needle assembly prior to insertion of the stimulation needle into the body tissue.

In some embodiments, the kit may further include a stylet insertable into an inner lumen of the needle. The stylet may comprise a hub providing electrical connection to the external source of stimulation, and the needle may not include an integral hub. When the stylet is fully inserted into the needle, a distal tip of the stylet may extend distally beyond the distal tip of the needle. The stylet may be removable from within the inner needle lumen to allow a distal end of the introducer assembly to be advanced over a proximal end of the elongate needle body.

Certain aspects of the disclosure relate to a stimulation needle assembly. The stimulation needle assembly may include a needle. The needle may comprise an elongate needle body having a proximal end and a distal tip. A length of the elongate needle body may be at least 150 mm. The needle may not include an integral hub at the proximal end of the elongate needle body. The needle may comprise an inner lumen with a diameter less than 0.5 mm, or may not comprise an inner lumen. An outer diameter of the elongate needle body may be at least 20 Gauge. The stimulation needle assembly may further comprise a hub configured to be removably coupled to the elongate needle body, wherein the hub is removable to allow an introducer assembly to be advanced over the elongate needle body. The stimulation needle assembly may be configured to be inserted into an individual and to deliver electrical stimulation to an individual from an external source to indicate that a distal tip of needle assembly is at a target location at or near a pudendal nerve. The elongate needle body may comprise an outer connection region disposed between proximal and distal insulated portions of the elongate needle body. The hub may be coupled to the outer connection region. The hub may be configured to electrically connect the needle to the external source of stimulation. The hub may comprise a locking mechanism configured to secure the hub to the needle. The locking mechanism may be reversible to remove the hub from the needle. In some embodiments, the stimulation needle assembly may further include a removable stylet comprising a stylet body and a stylet hub and configured to be inserted into an inner lumen of the elongate needle body. The stylet may be configured to deliver the electrical stimulation. The stylet body may be longer than the needle body such that a distal tip of the stylet extends distally beyond a distal tip of the needle when the stylet is fully inserted into the needle.

Certain aspects of the disclosure relate to a method of standardizing a pelvic tilt of a patient lying on an operating table during a lead implantation procedure in a pelvis. In some embodiments, the lead implantation procedure may be specific to the pudendal nerve, e.g., not for the sacral nerve. The method may include obtaining a medical image, e.g., an x-ray image, of the pelvis of the patient. The method may further include identifying a first bone landmark, a second bone landmark, and a third bone landmark corresponding to a first axis, a second axis, and a third axis, respectively, on the x-ray image of the pelvis. Each of the first axis, the second axis, and the third axis may be substantially parallel to each other. The second axis may be between the first axis and third axis. The method may further include determining a ratio of a first distance between the first axis and second axis to a second distance between the second axis and the third axis. The method may further include determining a first corrective action by comparing the ratio to a predetermined target ratio range. The first corrective action may comprise an adjustment to a head and/or foot end of the operating table. The method may further include causing an actuator to automatically adjust an orientation of the operating table according to the first corrective action. The steps of the method may be performed automatically by a computing system. However, certain steps of the method may be performed by a clinician. The first bone landmark may comprise a lower margin of a sacroiliac joint. The second bone landmark and the third bone landmark may comprise the upper and lower borders of an obturator foramen, respectively. One or more of the first bone landmark, the second bone landmark, and the third bone landmark may be identified using a trained artificial intelligence model. The predetermined target ratio range may be 1 to 1.25. When the ratio is below the predetermined target ratio range, the corrective action may comprise lowering the head end of the operating table. When the ratio is above the predetermined target ratio range, the corrective action may comprise lowering the foot end of the table. The method may further include identifying a fourth bone landmark and a fifth bone landmark on the x-ray image. The fourth bone landmark and the fifth bone landmark may be corresponding bone structures on a left and right side of the x-ray image. One or both of the fourth bone landmark and the fifth bone landmark may be identified using a trained artificial intelligence model. The fourth bone landmark and the fifth bone landmark may comprise the left and right obturator foramen, respectively. The method may further include determining a second corrective action based on a comparison of the fourth landmark and the fifth landmark. The comparison may be a size comparison. When the fourth landmark is bigger than the fifth landmark, the second corrective action may comprise lowering a right side of the operating table. When the fourth landmark is smaller than the fifth landmark, the second corrective action may comprise lowering a left side of the operating table. The method may further include causing the actuator to automatically adjust the orientation of the operating table according to the second corrective action. The pelvic tilt may be between about −10 to −15 degrees after causing the actuator to automatically adjust the operating table. The x-ray image may be in an anterior-posterior (AP) view. The method may further include implanting an electrode lead near a pudendal nerve of the patient. The electrode lead may be implanted near the pudendal nerve using an ischiorectal approach.

Certain aspects of the disclosure relate to a method of standardizing a pelvic tilt of a patient lying on an operating table during a lead implantation procedure in a pelvis. In some embodiments, the lead implantation procedure may be specific to the pudendal nerve, e.g., not for the sacral nerve. The method may include obtaining a medical image, e.g., an x-ray image, of the pelvis of the patient. The method may include identifying a first bone landmark and a second bone landmark on the x-ray image of the pelvis. The first and second bone landmarks may be corresponding bone structures on a left and right side of the x-ray image, respectively. The method may include determining a size difference between the first and second bone landmarks. The method may include determining a corrective action by comparing the size difference of the first bone landmark and the second bone landmark to a predetermined threshold. The corrective action may comprise a left or right side rotation of the operating table. The method may further include causing an actuator to automatically adjust an orientation of the operating table according to the corrective action. The first bone landmark and the second bone landmark may comprise a left and right obturator foramen, respectively. One or both of the first bone landmark and the second bone landmark may be identified using a trained artificial intelligence model. The size of the first bone landmark and the size of the second bone landmark may be identified using the trained artificial intelligence model. The trained artificial intelligence model may be configured to determine the size difference and/or the corrective action. When a size of the first bone landmark is smaller than a size of the second bone landmark, the corrective action may comprise lowering the left side of the operating table. When a size of the first bone landmark is larger than a size of the second bone landmark, the corrective action may comprise lowering the right side of the operating table. After causing the actuator to automatically adjust the operating table, a left-right rotational tilt of the operating table may be between about-10 to 10 degrees. The x-ray image may be in an anterior-posterior (AP) view. The method may further include implanting an electrode lead near a pudendal nerve of the patient.

Certain aspects of the disclosure relate to a non-transitory, computer-readable medium comprising computer-executable instructions for standardizing a pelvic tilt of a patient lying on an operating table during a lead implantation procedure. In some embodiments, the lead implantation procedure may be specific to the pudendal nerve, e.g., not for the sacral nerve. The computer-executable instructions, when executed by a computer system, may cause the computer system to obtain an x-ray image of the pelvis of the patient. The computer-executable instructions, when executed by a computer system, may further cause the computer system to identify at least a first bone landmark, a second bone landmark, and a third bone landmark and a corresponding first axis, second axis, and third axis on the x-ray image of the pelvis. The first bone landmark may comprise a lower margin of a sacroiliac joint of the patient. The second bone landmark and the third bone landmark comprise the upper and lower borders of an obturator foramen, respectively. Each of the first axis, second axis, and the third axis may be substantially parallel to each other. The second axis may be between the first axis and third axis. One or more of the first bone landmark, the second bone landmark, and the third bone landmark may be identified using a trained artificial intelligence model. The computer-executable instructions, when executed by a computer system, may further cause the computer system to determine a ratio of a first distance between the first axis and the second axis to a second distance between the second axis and the third axis. The computer-executable instructions, when executed by a computer system, may further cause the computer system to determine a first corrective action by comparing the ratio to a predetermined target ratio range. The predetermined target ratio range may be 1 to 1.25. The corrective action may comprise an adjustment to a head and/or foot end of the operating table. When the ratio is below the predetermined target ratio range, the corrective action may comprise lowering the head end of the operating table. When the ratio is above the predetermined target ratio range, the corrective action may comprise lowering the foot end of the table. The computer-executable instructions, when executed by a computer system, may further cause the computer system to cause an actuator to automatically adjust an orientation of the operating table according to the first corrective action.

Certain aspects of the disclosure relate to a needle assembly for determining a position for an electrode lead to be implanted near a pelvis of a patient. In some embodiments, the position for the electrode lead may be specific to stimulating the pudendal nerve only, e.g., not for stimulating the sacral nerve. The assembly may include a needle. The needle may include an elongate needle body comprising an outer connection region configured to interface with a source of electrical stimulation. The needle may include a needle grip comprising a grip body and two arms extending from the grip body. The needle grip may be positioned at or near the proximal end of the elongate needle body such that the two arms receive the needle body. The two arms of the needle grip portion may define the outer connection region of the needle body. The assembly may include a removable stylet comprising a elongate stylet body configured to be inserted through the needle grip portion and into a lumen of the elongate needle body. The outer connection region of the needle body may be configured to interface with the source of electrical stimulation via a J-clip. The needle may comprise a stimulating needle configured to output electrical stimulation provided by the source of electrical stimulation. The removable stylet may comprise a stylet grip having a same cross-sectional shape as one of the two arms of the needle grip. One, or both, of the two arms of the needle grip may taper from the grip body and toward the elongate needle body. The assembly may further include a needle guide comprising a guide body comprising a plurality of channels formed within the guide body. Any channel of the plurality of channels may be configured to receive the needle. At least some of the plurality of channels may be arranged in an array. Each channel of the plurality of channels may be substantially parallel to each other. Each channel of the plurality of channels may have an proximal opening on a proximal side of the guide body and a distal opening on an opposite distal side of the guide body. The assembly may further include a plurality of needles. The plurality of needles may each be identical to the needle. Each needle of the plurality of needles may be configured to be inserted into a corresponding channel of the plurality of channels.

Certain aspects of the disclosure relate to a kit for determining a position for an electrode lead to be implanted near a pelvis of a patient. In some embodiments, the position for the electrode lead may be for stimulating a pudendal nerve only, e.g., not for stimulating a sacral nerve. The kit may include a needle guide comprising a guide body. The guide body may comprise a plurality of channels formed within the guide body. Each channel of the plurality of channels may have a proximal opening on a proximal side of the guide body and a distal opening on an opposite distal side of the guide body. The kit may further include a plurality of needles. Each of the plurality of needles may comprise a needle grip and an elongate needle body. Each needle body of the plurality of needles may be configured to be inserted through a corresponding channel of the needle guide. Each needle grip may comprise a grip body tapered towards a tapered end holding the elongate needle body. The needle grips may be sized to not interfere with each other when the plurality of needles are inserted into adjacent channels of the needle guide. Each of the plurality of channels of the needle guide may be substantially parallel to each other. Each of the plurality of channels may be equidistant to adjacent ones of the channels. Each of the plurality of channels may be positioned no more than 3 mm away from adjacent ones of the channels. The needle guide may comprise a T-handle configured to be gripped by a user. The needle guide may be configured to receive up to eight needles simultaneously. When the plurality of needles are positioned in the needle guide, the needle grips of the plurality of needles may be arranged circumferentially with the tapered ends of the needle grips facing toward each other. The elongate needle body of each of the plurality of needles may comprise an outer connection region configured to interface with a source of electrical stimulation. The outer connection region of the elongate needle body may be defined by two arms of the needle grip disposed at each end of the connection region. The outer connection region may be configured to interface with the source of electrical stimulation via a J-clip. Each of the plurality of needles may include a removable stylet configured to be inserted into an inner lumen of the needle.

Certain aspects of the disclosure relate to a method of determining an optimal position near a pelvis of an individual for lead placement using the kit comprising the needle guide and a plurality of needles. In some embodiments, the optimal position for lead placement may be for stimulating a pudendal nerve only, e.g., not for stimulating a sacral nerve. The method may include inserting the plurality of needles through the needle guide and into the individual near the pelvis. The method may include delivering electrical stimulation through each of the plurality of needles one by one to elicit one or more bioelectrical responses from the individual. The method may include determining an optimal needle position based on a comparison of the bioelectrical responses elicited from each of the plurality of needles. The electrical stimulation may be delivered to a pudendal nerve.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative non-limiting embodiments, in which the principles of the disclosure are utilized, and the accompanying drawings described below. Features from one figure may be combined with features of other figures.

FIGS. 1A-1J show an embodiment of the electrode lead introducer, as described in some embodiments herein.

FIGS. 2A-2G show an embodiment of the anchor in various views or detailed views, as described in some embodiments herein.

FIGS. 3A-3D show an embodiment of a plurality of anchors in a closed and/or compressed state on an electrode lead, as described in some embodiments herein.

FIGS. 4A-4D show an embodiment of a plurality of anchors in an open and/or splayed state on an electrode lead, as described in some embodiments herein.

FIG. 5 shows an embodiment of an electrode lead and the spacing of the one or more anchors on the electrode lead, as described in some embodiments herein.

FIGS. 6A-6C show an embodiment of a serrated (FIGS. 6A-6B) barb anchor and an anchor with a triangular profile, as described in some embodiments herein.

FIGS. 7A-7B show an embodiment of a molded anchor, as described in some embodiments herein.

FIGS. 8A-8C show an embodiment of a plurality of anchors on an electrode with a pusher and an introducer elongated body, as described in some embodiments herein.

FIGS. 9A-9C show an embodiment of a plurality of anchors on an electrode lead in a compressed or closed state as the electrode lead and the plurality of anchors are pushed through the introducer elongated body lumen with the pusher, as described in some embodiments herein.

FIGS. 10A-10C show an embodiment of a plurality of anchors on an electrode lead exiting the introducer elongated body lumen as the introducer is removed and/or pulled back over the pusher, electrode lead, and the plurality of anchors on the electrode lead, as described in some embodiments herein.

FIG. 11 shows a flow of affixing an electrode lead to a tissue, as described in some embodiments herein.

FIG. 12 shows a flowchart of the steps in for performing electrode lead and implantable pulse generator (IPG) implantation, as described in some embodiments herein.

FIG. 13 shows a schematic of the anatomy and the disposition of leads and IPG in an individual, as described in some embodiments herein.

FIG. 14 shows a schematic of the anatomy and implanted leads and IPG in an individual, as described in some embodiments herein.

FIG. 15 show schematics of the anatomy and implanted leads and IPG in an individual, as described in some embodiments herein.

FIGS. 16A and 16B show embodiments of the anatomical paths of the leads using anatomical models, as described in some embodiments herein.

FIG. 17 shows an embodiment of a lead positioning guide (LPG), as described in some embodiments herein.

FIG. 18A-18C show an embodiment of securing a lead in a lead positioning guide (LPG) with a locking cap, as described in some embodiments herein.

FIG. 19 shows an embodiment of a lead positioning guide (LPG) and sheath handle with a small overhang configuration, as described in some embodiments herein.

FIG. 20 shows an embodiment of a lead positioning guide (LPG) and sheath handle with an extended overhang configuration, as described in some embodiments herein.

FIG. 21 shows an embodiment of a lead positioning guide (LPG) and sheath handle with a curved face overhang configuration, as described in some embodiments herein.

FIG. 22 shows an embodiment of a lead positioning guide (LPG) and sheath handle with a pin lock configuration, as described in some embodiments herein.

FIGS. 23A and 23B show embodiments of a lead positioning guide (LPG) and sheath handle with a broad lock configuration, as described in some embodiments herein.

FIGS. 24A-24K show embodiments of positioning a lead with an anchoring device using a lead positioning guide (LPG) and introducer, as described in some embodiments herein.

FIG. 25 shows embodiments of a helical lead body, as described in some embodiments herein.

FIG. 26 shows an embodiment of positioning a lead or needle using an obturator and introducer, as described in some embodiments.

FIG. 27 shows a schematic of surface markings drawn on an individual to locate the pudendal nerve, as described in some embodiments herein.

FIG. 28 shows a schematic of a lateral view of the anatomy and an inserted marking needle in an individual, as described in some embodiments herein.

FIG. 29 shows a schematic of a bilateral lead placement at the pudendal nerves, as described in some embodiments herein.

FIGS. 30A-30C show embodiments of a modular anchor assembly and a spiral anchor assembly, as described in some embodiments herein.

FIG. 31 shows an embodiment of an anchor comprising a mating feature, as described in some embodiments herein.

FIG. 32 shows an embodiment of a target pelvic tilt on an operating table, as described in some embodiments herein.

FIG. 33 shows an embodiment of an anterior-posterior (AP) view model of a pelvis with identified landmarks, as described in some embodiments herein.

FIG. 34 shows an embodiment of an AP view x-ray image of a pelvis with markings identifying bone landmarks, as described in some embodiments herein.

FIG. 35 shows an embodiment of an AP view x-ray image where no corrective action is required, as described in some embodiments herein.

FIGS. 36-40 show embodiments of AP view x-ray images where corrective action is required, as described in some embodiments herein.

FIG. 41 shows a needle and a removable stylet, as described in some embodiments herein.

FIG. 42 shows a needle grip with a connection region, as described in some embodiments herein.

FIGS. 43A-43C show different embodiments of a needle guide, as described in some embodiments herein.

FIG. 44 shows one or more needles inserted into a needle guide, as described in some embodiments herein.

FIG. 45 shows another embodiment of one or more needles inserted into a needle guide, as described in some embodiments herein.

FIGS. 46A and 46B show an embodiment of a stimulation needle and introducer assembly, as described in some embodiments herein.

FIG. 47 shows an embodiment of a stimulation needle with an outer connection region distal to a proximal end, as described in some embodiments herein.

FIG. 48 shows an embodiment of a removable hub for a stimulation needle assembly, as described in some embodiments herein.

### DETAILED DESCRIPTION

Electrical stimulation has clinical application in providing treatment and/or management of a variety of clinical conditions, for example within the pelvic region. For example, electrical stimulation may be used to treat urinary incontinence (such as urge, stress, or mixed urinary incontinence), fecal incontinence, pain, sexual dysfunction, or any combination thereof medical condition and/or diseases within the pelvic region.

The devices, systems, methods, and/or kits described elsewhere herein, provide solutions for implanting a treatment device for treating incontinence. Incontinence, including a lack of control over micturition or bowel movements, has many causes but may involve injury or weakness of the pelvic floor muscles and the nerves that innervate these muscles and involved organs. Several embodiments described herein provide systems and methods for implanting a treatment device to treat various conditions, such as urinary incontinence, fecal incontinence, pain, sexual dysfunction, or any combination thereof medical condition and/or diseases within the pelvic region. In some embodiments, the pelvic condition includes urinary incontinence, fecal incontinence, pain, sexual dysfunction, or any combination thereof. Although several embodiments are described herein with respect to the pelvic region to treat and/or manage pelvic conditions, they may also be used in other regions of the body or to treat other conditions as described elsewhere herein. With respect to urinary incontinence, the treatment device may treat one or more types of incontinence (e.g., urge, stress, mixed).

Although certain devices, systems, methods, and kits are described herein with respect to the pelvic region to treat and/or manage pelvic conditions, the methods and devices can be used in other regions of the body or to treat other conditions. The anchors may be used to fix implantable devices in other regions of the body and/or to treat other conditions. In some embodiments, the anchors may be applied to electrode lead bodies implanted in the spinal region, for example to treat chronic or incidental pain. The anchors may be applied to electrode lead bodies in or near the spine to treat, for example, pain (e.g., the spinal cord region caudal of the occiput or other spinal regions). In other embodiments, the anchors may be applied to electrode lead bodies implanted near peripheral or cranial nerves. For example, the anchors may be applied to an electrode lead body implanted near the vagus nerve (e.g., in the facial/cranial region) for treatment of several disorders, including but not limited to balance issues, headache, migraines, etc. In some embodiments, the anchors may be applied to an electrode lead body used for peripheral nerve stimulation at any location in the body. In some embodiments, the anchors may be applied to implantable devices used outside of the field of neuromodulation. For example, the anchors may be applied to sutures used in soft tissue repair (e.g., rotator cuff repair) in place of clips. In some embodiments, the anchors may be applied to non-implantable or acute devices such as catheters, for example drug delivery catheters or drainage catheters.

Electrode leads may be implanted at one or more target tissue and/or anatomical features, for example in the pelvic region, to provide treatment and/or management electrical stimulation. However, implanted electrode lead(s) may experience axial tensile forces, compressive forces, torque, bending force, or any combination thereof forces, during activities of deep flexion or femoral rotation caused by e.g., motion from sitting to standing, moving up or down stairs, and/or movement while sleeping, particularly in the pelvic region due to the anatomy of the pelvis. Moreover, the implanted electrode leads also experience compressive forces from surrounding soft tissues in instances where a subject with an implanted electrode is sitting or lying supine. The forces acted upon the electrode lead may migrate and/or displace the electrode lead from its target implantation location within the pelvis thus diminishing the treatment provided by electrical stimulation to the target implantation site. The devices, systems, methods, and/or kits described elsewhere herein, provide a solution to fix and/or anchor the electrode lead at the target implantation location. The devices, systems, methods, and/or kits, as described elsewhere herein, may comprise one or more anchors with one or more barbs that may fix and/or prevent unwanted displacement of the electrode lead from its targeted implanted location. The orientation and/or rotational angle of the one or more anchors and/or the one or more barbs, as described elsewhere herein, may provide better than expected fixation of the electrode lead at or nearby spatially heterogenous tissues e.g., ligaments and/or other connective tissue within the pelvis where the electrode lead is implanted.

Provided herein are devices, systems, methods, and/or kits for accessing a target tissue, for example in the pelvic cavity, and anchoring an electrical lead at the targeted tissue for treatment by electrical nerve stimulation. The tissue may comprise connective tissue, nerve tissue, muscle tissue, ligament issue, fascia tissue, fat, or any combination thereof. The connective tissue may comprise tissue of a sacrotuberous ligament, sacrospinous ligament, fascial and periosteal tissues of the falciform process, other dense tissues in the region medial to the ischial tuberosity, or any combination thereof. The target tissue may comprise a pudendal nerve or a tissue adjacent the pudendal nerve. Described herein are devices, systems, methods, and/or kits for providing electrical nerve stimulation to prevent an episode of incontinence, treat pain, treat sexual dysfunction, or any combination thereof, in an individual in need thereof. The devices, systems, and methods for placing an electrical lead to a target nerve site may comprise an introducer sheath, an obturator, and a needle, and/or a pusher, described elsewhere herein. The pusher may also be referred herein as a lead positioning guide (LPG) or an anchor positioning guide (APG). The pusher, also referred herein as LPG or APG, may be used to stabilize the position or produce axial (push) force to the anchor when the sheath is removed. The introducer sheath may comprise a sheath elongate shaft having a lumen, a sheath handle at a distal end of the elongated body, wherein the introducer sheath is configured to receive the electrode on an outer surface of the sheath elongate shaft. The obturator may comprise an obturator elongate shaft having a lumen and an obturator handle at a distal end of the obturator elongate shaft, wherein the obturator elongate shaft is configured to fit inside the sheath lumen. The needle may comprise a needle elongate shaft, a needle handle at a distal end of the needle elongate shaft, and a needle tip at the proximal end of the needle elongate shaft, wherein the needle elongate shaft is configured to fit inside the obturator lumen. The devices, systems, and methods for placing an electrical lead to a target tissue described herein may allow for easier access to the target tissue and for an accurate electrode placement despite complex three-dimensional anatomy in the pelvic region. The use of a closed loop and/or feed forward algorithm stimulation of the electrode lead may reduce or minimize issues with issues of stimulation tolerability, which may reduce the effectiveness of nerve stimulation (e.g., PNS) over time. The devices, systems, methods and/or kits provided herein may be compatible with electrophysiological guidance, alone or in combination with radiological guidance, for accurate and reproducible placement of electrodes at a target tissue (e.g., a pudendal nerve). The devices, systems, and methods provided herein may allow for more accurate and reproducible placement of electrodes on the difficult-to-access tissues of the pelvic cavity that vary less with the skill of the healthcare professional performing the procedure. In some implementations, robotic arm(s) and/or robotic control may be used for the surgical procedure. In some cases, stereotactic guidance may be used for manually and/or robotically-assisted placement and/or manipulation of the instruments and/or implants during the procedure.

Described herein are devices, systems, methods, and/or kits for accessing a region, for example the pelvic region, in a subject to place and fix an electrical lead on to a target tissue. The electrical lead may provide electrical stimulation to the target tissue to treat a pelvic condition. The pelvic condition may comprise urinary incontinence, fecal incontinence, pain, sexual dysfunction, voiding dysfunction, or any combination thereof. The devices, systems, methods, and/or kits described herein may provide electrical nerve stimulation to prevent an episode of incontinence in an individual in need thereof. The devices, systems, and methods provided herein may access the pudendal nerve by an ischiorectal approach. In cases, the ischiorectal approach may comprise the lead introducer penetrating or passing close to the sacrotuberous ligament and directing the lead to the pudendal nerve trunk at a location proximal to Alcock's canal. The devices, systems, and methods provided herein may access the pudendal nerve by a low gluteal approach, also referred herein as a low posterior approach. The low gluteal approach may comprise where the lead introducer and the lead passes in a space between sacrotuberous and sacrospinous ligaments and passes anteriorly in the ischiorectal fossa below the pelvic floor. The lead placed using the low gluteal approach may stimulate the anterior branches of the pudendal nerve (including the dorsal genital nerve).

### Electrode Lead Introducer

Provided herein are devices, systems, and methods for introducing and placing one or more electrode leads at one or more target tissues, for example within the pelvic region. The target tissue may comprise a pudendal nerve to treat incontinence. The target tissue may comprise a target tissue to receive electrical stimulation for sexual dysfunction. The target tissue may comprise a target tissue to receive electrical stimulation for pain treatment and/or management. Although certain devices, systems, methods, and kits are described herein with respect to the pelvic region to treat and/or manage pelvic conditions, the methods and devices can be used in other regions of the body or to treat other conditions as described elsewhere herein.

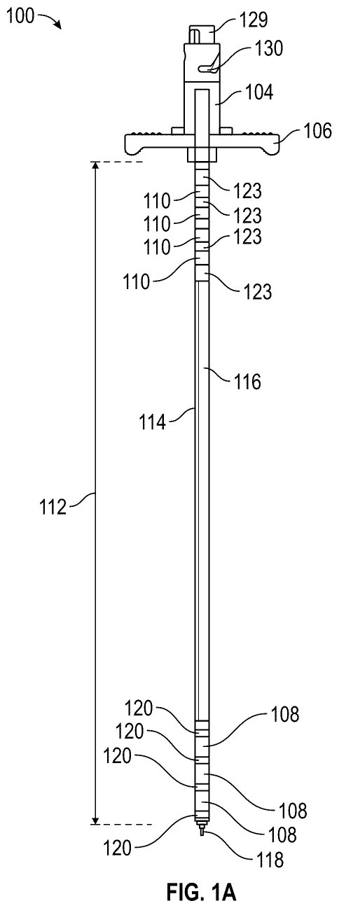

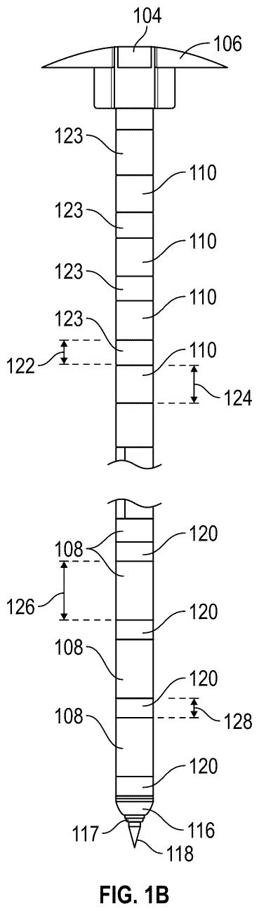

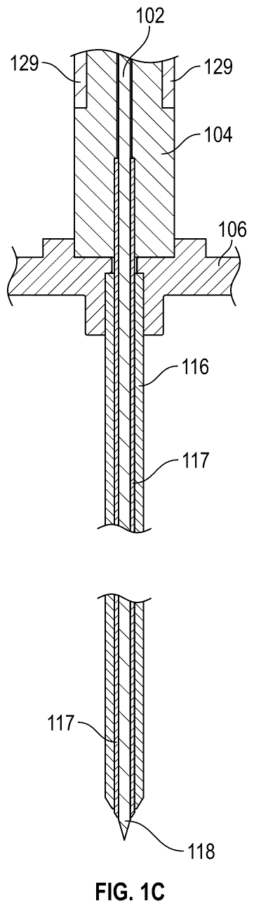

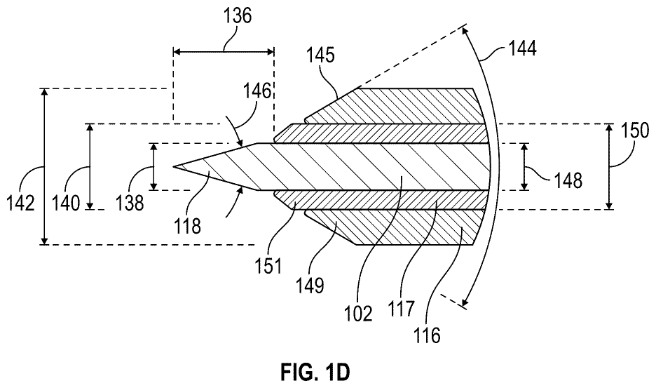

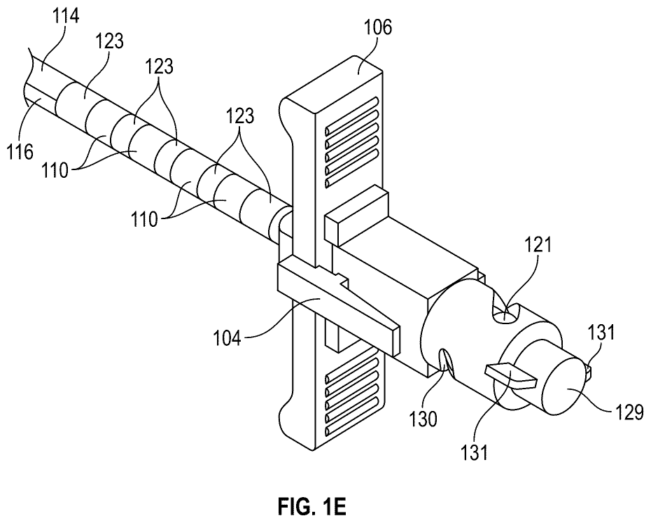

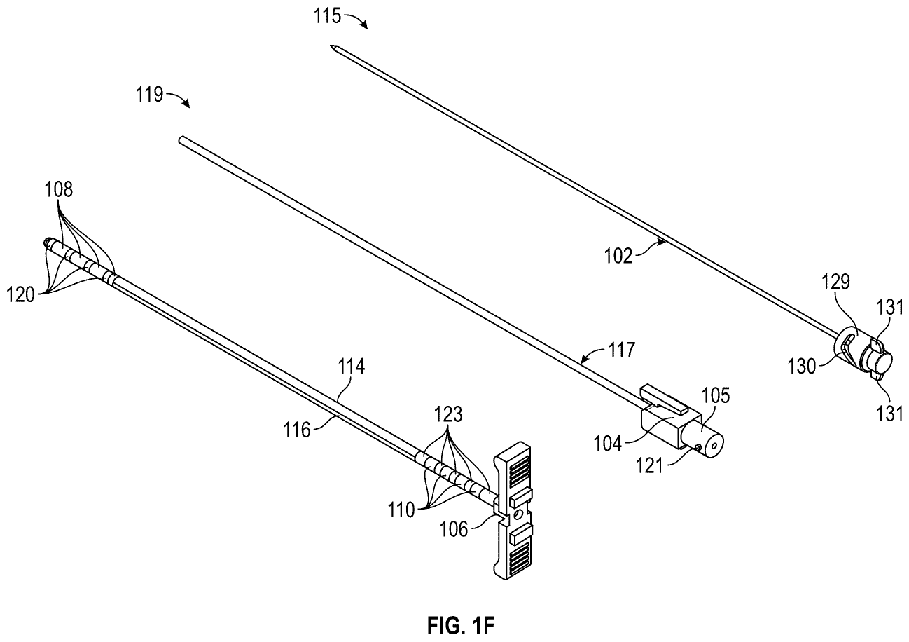

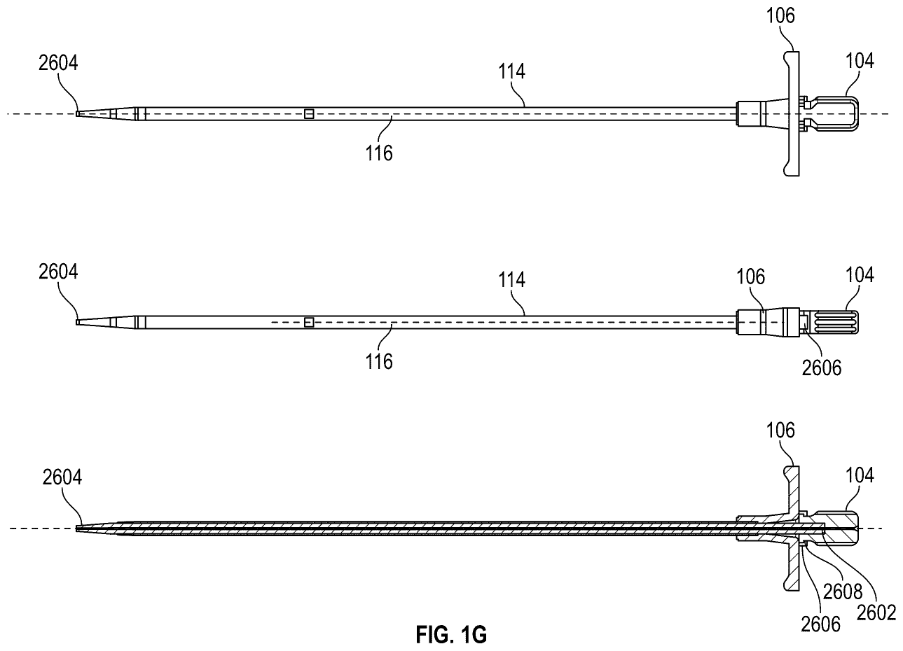

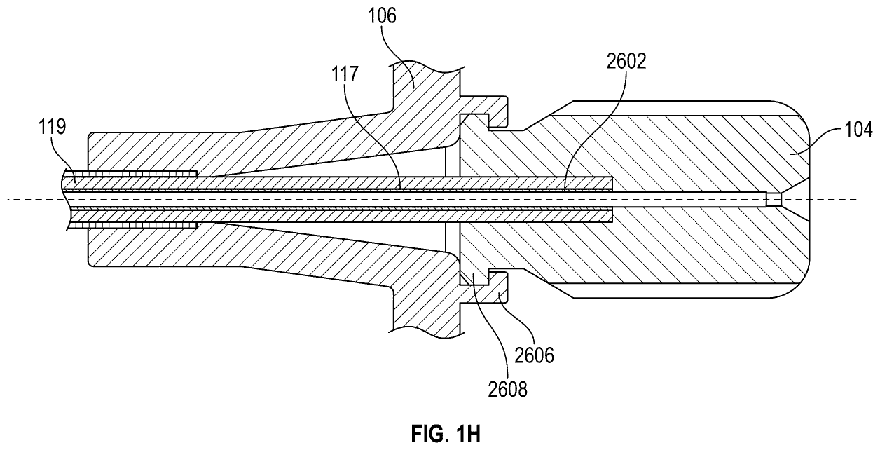

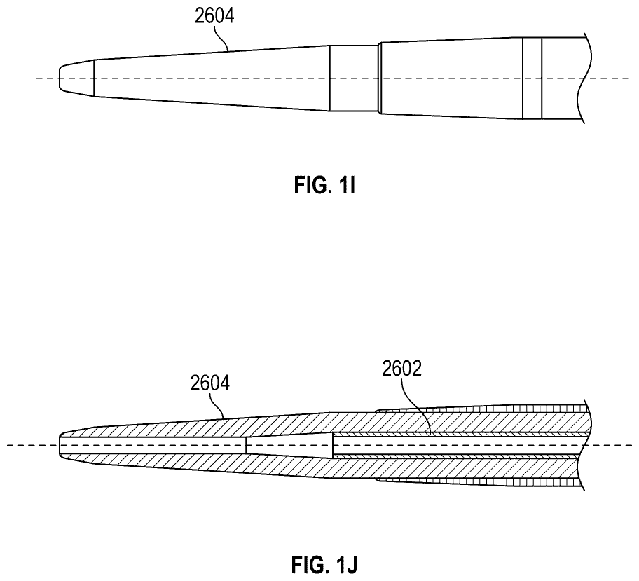

An electrode lead introducer used for placing the electrode at the target tissues may comprise an introducer sheath, a dilator (also referred herein as an obturator), and a needle. FIGS. 1A-1F show an embodiment of the electrode lead introducer 100. The electrode lead introducer may comprise an introducer sheath 114, an elongated body 116, an obturator 119, a needle 115, one or more electrodes (110, 120), one or more regions of electrode insulation (108, 123) or any combination thereof. The elongated body 116 may be partially or wholly covered, coated, or surrounded by the introducer sheath 114.

The introducer sheath may comprise a sheath elongate shaft having a lumen and a sheath handle at a distal end of the sheath shaft. The introducer sheath may be configured to receive the electrode on an outer surface of the sheath elongate shaft. In some embodiments, a proximal end of the sheath elongate shaft may be angled. The angle of the proximal end of the sheath elongate shaft may allow for advancing the device with little to no damage to surrounding tissue. The introducer sheath may have a diameter ranging from about 0.5 mm to about 5 mm.

The obturator may comprise an obturator elongate shaft having a lumen and an obturator handle at a distal end of the obturator elongate shaft. The obturator elongate shaft may be configured to fit inside the sheath lumen. The obturator may have a diameter ranging from about 0.5 mm to about 5 mm.

The needle may comprise a needle elongate shaft, a needle handle at a distal end of the needle elongate shaft, and a needle tip at the proximal end of the needle elongate shaft. The needle elongate shaft may be configured to fit inside the obturator lumen. The obturator handle may comprise a latch configured to attach to the sheath handle. In some embodiments, the needle tip may be configured to protrude beyond an end of the obturator lumen. The needle tip may protrude at least 1 mm beyond the end of the obturator lumen. The needle tip may be configured to protrude by a movement of the needle handle. The needle tip may be configured to be retractable into the obturator lumen. The needle tip may be angled from the needle elongate shaft. The needle tip angle may be configured to advance the needle tip through a soft tissue. The needle may have a diameter of about 0.4 mm to about 2 mm. In some embodiments, the needle may have a diameter of between 12 and 26 gauge.

The elongated body 116 may be attached, fastened, and/or fused to a sheath handle 106 at one end of the elongated body 116, also referred herein as elongated shaft, as shown in FIGS. 1A, 1C, 1E, and 1F. The introducer sheath 114 may be integrated with the one or more electrodes and/or conductive regions (110, 120) and/or one or more regions of electrode insulation (108, 123). In some embodiments, the introducer sheath 114 may cover or surround, in whole or in part, non-conducting regions (108, 123) of one or more electrodes (110,120) integrated within the elongated body 116. The introducer sheath 114 may comprise a non-conductive biocompatible material, including but not limited to high-density polyethylene (HDPE), fluorinated ethylene propylene (FEP), polycarbonate, plastics, or any combination thereof. In some embodiments, at least a portion of the introducer sheath 114 may comprise a radiopaque additive, including but not limited to barium sulfate (BaSO₄, bismuth subcarbonate (BiO)₂CO₃, bismuth oxychloride (BiOCl), bismuth trioxide (Bi₂O₃), or tungsten (W). For example, the material used for the tip of the introducer sheath 114 may comprise barium sulfate (BaSO₄) additive so a user, medical personnel, and/or surgeon may visualize the tip within the patient's body. In some instances, there may be one or more radiopaque markers disposed on the introducer sheath 114. In some instances, the introducer sheath 114 may be single use and/or disposable. The introducer sheath 114 may be autoclavable and/or may be cleaned by conventional sterilization methodologies used for other similar medical devices (i.e., obturators, trocars, endoscopes, etc.).

The sheath handle 106 may be configured to allow a user, medical personnel, and/or a surgeon to manipulate and/or navigate the electrode lead inserter as it is advanced into a patient or subject. The sheath handle 106 may comprise an ergonomic geometry, where such ergonomic geometry may be configured to be operated with a single hand of a user, medical personnel, and/or a surgeon, freeing up the other hand of the user, medical personnel, and/or surgeon for other tasks. Although the techniques described herein may be performed manually, in other embodiments, the instruments may be incorporated by or controlled by robotic systems and/or facilitated using augmented reality.

The mechanical stiffness of the material for the elongated body 116 and the introducer sheath 114 may be chosen to allow for easy insertion of the electrode lead introducer 100 into a patient. The Young's modulus of the introducer sheath 114 and the elongated body 116 may allow for a user, medical personnel, and/or surgeon to maneuver the electrode lead inserter into a deep surgical plane of the pelvic region. The Young's modulus of the introducer sheath 114 and/or the elongated body 116 may prevent bowing or flexing of the combined elongated body 116 and introducer sheath when the user, medical personnel, and/or surgeon exerts a force on the distal end of the device when inserting into a patient. The Young's modulus of the introducer sheath 114 and/or the elongated body 116 may lower the total mechanical work necessary to insert the electrode lead introducer into deep muscle and/or fat surgical planes in the pelvic region, while maintaining the position of the guidewire. The flexibility of the assembly allows the introducer and lead to follow the course of the guidewire without disturbing the position close to the nerve. A higher stiffness of the sheath may allow for easier delivery of the lead in regions with high tissue density or tissue resistance. A higher stiffness of the sheath may allow for more accurate placement of the lead to the target site in regions with high tissue density or tissue resistance around the target site. The stiffness of the materials may be characterized by Young's modulus. The introducer sheath 114 may have a Young's modulus of about 10 mega pascals (MPa) to about 10,000 MPa. The elongated body 116 may have a Young's modulus of about 10 MPa to about 10,000 MPa. The sheath of the introducer may have a higher stiffness than a sheath of an introducer typically used to access a sacral nerve. The higher stiffness may allow for easier access to the target tissue e.g., a pudendal nerve and easier electrode lead placement on target anatomy. The combination of the sheath and needle may have a stiffness similar to the combined sheath and dilator of an introducer typically used to access the sacral nerve. In some instances, the combination of the sheath and needle of the introducer may have a higher stiffness than the combined sheath and dilator of an introducer typically used to access the sacral nerve.

The elongated body 116 may comprise a length 112. The length 112 of the device may enable proper manipulation of the device within patients comprising varying anatomical features, to properly place the one or more electrode leads, as described elsewhere herein. The length of the elongated body may refer to an insertable length. In some cases, the anatomical feature variations between subjects may comprise an enlargement or reduction in anatomical features surrounding or adjacent the pudendal, sacral nerves, or any combination or branches thereof.

The length of the elongated body 116 may comprise a distance from about 10 centimeters (cm) to about 20 cm. For example, the length of the elongated body 116 may comprise a distance from about 12 cm to about 20 cm, about 13 cm to about 20 cm, or about 14 cm to about 20 cm. The length of the elongated body 116 may comprise a distance of about 12 cm, about 13 cm, about 14 cm, about 15 cm, about 16 cm, about 17 cm, about 18 cm, about 19 cm, or about 20 cm. In some cases, the length of the elongated body 116 may comprise a distance of at least about 12 cm, about 13 cm, about 14 cm, about 15 cm, about 16 cm, about 17 cm, about 18 cm, or about 19 cm. In some cases, the length of the elongated body 116 may comprise a distance of at most about 13 cm, about 14 cm, about 15 cm, about 16 cm, about 17 cm, about 18 cm, about 19 cm, or about 20 cm.

FIG. 1D shows, in some embodiments, the elongated body 116, comprising an inner lumen diameter 150 that is configured to allow for the obturator 119 outer diameter 140 to pass through the inner lumen of the elongated body with a slip-fit mechanical interface.

The elongated body inner lumen diameter 150 may be about 0.5 mm to about 3 mm. In some cases, the elongated body inner lumen diameter 150 may comprise a distance of about 0.5 to about 4 mm, or about 0.5 mm to about 5 mm. The elongated body inner lumen diameter 150 may comprise a distance of about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 2 mm, about 2.5 mm, or about 3 mm. In some cases, the elongated body inner lumen diameter 150 may comprise a distance of at least about 1.2 mm, about 1.3 mm, about 1.4 mm, about 1.5 mm, about 1.6 mm, about 1.7 mm, about 1.8 mm, about 2 mm, or about 2.5 mm. In some cases, the inner lumen diameter 150 may comprise a distance of at most about 1.3 mm, about 1.4 mm, about 1.5 mm, about 1.6 mm, about 1.7 mm, about 1.8 mm, about 2 mm, about 2.5 mm, or about 3 mm. In some cases, the elongated body inner lumen diameter 150 may be a diameter that accept a guidewire such that the guidewire and the inner lumen diameter 150 comprise a slip-fit mechanical interface.

The elongated body outer diameter 142 may comprise a diameter of about 0.5 mm to about 10 mm. For example, the elongated body outer diameter 142 may comprise a diameter of about 1 mm to about 10 mm, about 2 mm to about 10 mm, or about 3 mm to about 10 mm. The elongated body outer diameter 142 may comprise a diameter of about 0.5 mm, about 1 mm, about 1.5 mm, about 2 mm, about 2.5 mm, about 3 mm, about 3.5 mm, about 4 mm, about 4.5 mm, about 5 mm, about 6 mm, about 8 mm, or about 10 mm. In some cases, the elongated body outer diameter 142 may comprise a diameter of at least about 1 mm, about 1.5 mm, about 2 mm, about 2.5 mm, about 3 mm, about 3.5 mm, about 4 mm, about 4.5 mm, about 5 mm, about 6 mm, or about 8 mm. In some cases, the elongated body outer diameter 142 may comprise a diameter of at most about 1.5 mm, about 2 mm, about 2.5 mm, about 3 mm, about 3.5 mm, about 4 mm, about 4.5 mm, about 5 mm, about 6 mm, about 8 mm, or about 10 mm.

The elongated body 116 may comprise an angled facet 145 on one end of the elongated body 116, as shown in FIG. 1D. The angled facet 145 of the elongated body 116 may be configured to allow for the electrode lead introducer 100 to penetrate into a subject receiving an implanted electrode in a similar manner as a pointed needle. The angle facet 145 of the proximal end of the sheath elongate shaft may allow for advancing the device with little to no damage to surrounding tissue. The angled facet 145 may be angled at an angle 144 with respect to a mirrored angled facet 149 of the elongated body 116 separated by 180 degrees.

The angle 144 may comprise a value of about 30 degrees to about 90 degrees. For example, the angle 144 may comprise a value of about 45 degrees to about 90 degrees, or about 60. The angle 144 may comprise a value of about 30 degrees, about 35 degrees, about 40 degrees, about 45 degrees, about 50 degrees, about 55 degrees, about 60 degrees, about 65 degrees, about 70 degrees, about 75 degrees, about 80 degrees, or about 90 degrees. In some cases, the angle 144 may comprise a value of at least about 30 degrees, about 35 degrees, about 40 degrees, about 45 degrees, about 50 degrees, about 55 degrees, about 60 degrees, about 65 degrees, about 70 degrees, about 75 degrees, or about 80 degrees. In some cases, the angle 144 may comprise a value of at most about 35 degrees, about 40 degrees, about 45 degrees, about 50 degrees, about 55 degrees, about 60 degrees, about 65 degrees, about 70 degrees, about 75 degrees, about 80 degrees, or about 90 degrees.

The obturator 119 may comprise an obturator elongated body 117 having a lumen and an obturator handle 104 at an end of the obturator elongated body, as shown in FIG. 1F. The obturator elongated body 117 may be comprised of a plastic (e.g., ABS), metal, or any combination thereof. For example, the metal may comprise stainless steel, aluminum, titanium, or any combination thereof. The obturator may be comprised of a stainless-steel, aluminum, titanium, or any combination thereof material for the obturator elongated body 117, yet comprise a plastic handle 104. The obturator elongated body 117 may be configured to fit inside the elongated body 116 lumen.

The obturator 119 may further comprise a stiffener tube 2602, as shown in FIGS. 1G and 1J. The stiffener tube 2602 may be comprised of a plastic (e.g., ABS), metal, or any combination thereof. For example, the stiffener tube 2602 may be comprised of stainless steel. The stiffener tube 2602 may run down the center of the elongated body 117. In some cases, the stiffener tube may be molded into the elongated body 117. The stiffener tube 2602 may be configured to improve the rigidity of the obturator 119. The stiffener tube may improve control and positioning of the obturator 119. The obturator 119 may comprise a tapered distal tip 2604.

The obturator elongated body 117 may comprise an outer diameter 140. The outer diameter 140 of the obturator elongated body 117 may comprise a diameter of about 0.5 mm to about 5 mm. For example, the outer diameter 140 of the obturator elongated body 117 may comprise a diameter of about 0.6 mm to about 5 mm, or about 1 mm to about 5 mm. The outer diameter 140 of the obturator elongated body 117 may comprise a diameter of about 0.5 mm, about 0.6 mm, about 0.7 mm, about 0.8 mm, about 0.9 mm, about 1 mm, about 1.5 mm, about 2 mm, about 2.5 mm, about 3 mm, about 4 mm, or about 5 mm. In some cases, the outer diameter 140 of the obturator elongated body 117 may comprise a diameter of at least about 0.5 mm, about 0.6 mm, about 0.7 mm, about 0.8 mm, about 0.9 mm, about 1 mm, about 1.5 mm, about 2 mm, about 2.5 mm, about 3 mm, or about 4 mm. In some cases, the outer diameter 140 of the obturator elongated body 117 may comprise a diameter of at most about 0.6 mm, about 0.7 mm, about 0.8 mm, about 0.9 mm, about 1 mm, about 1.5 mm, about 2 mm, about 2.5 mm, about 3 mm, about 4 mm, or about 5 mm.

The obturator 119 may comprise an inner lumen, as seen in FIG. 1D. The inner lumen may comprise an inner diameter 148. The inner lumen of the obturator 119 may comprise a diameter such that the needle body 102 and obturator inner lumen may be mechanically coupled with a slip fit interface.

The inner diameter 148 of the inner lumen of the obturator 119 may comprise a diameter of about 0.2 mm to about 1.4 mm. For example, the inner diameter 148 of the inner lumen of the obturator 119 may comprise a diameter of about 0.3 mm to about 1.4 mm, or about 0.5 mm to about 1.4 mm. The inner diameter 148 of the inner lumen of the obturator 119 may comprise a diameter of about 0.2 mm, about 0.3 mm, about 0.4 mm, about 0.5 mm, about 0.6 mm, about 0.7 mm, about 0.8 mm, about 0.9 mm, about 1 mm, about 1.2 mm, or about 1.4 mm. In some cases, the inner diameter 148 of the inner lumen of the obturator 119 may comprise a diameter of at least about 0.2 mm, about 0.3 mm, about 0.4 mm, about 0.5 mm, about 0.6 mm, about 0.7 mm, about 0.8 mm, about 0.9 mm, about 1 mm, or about 1.2 mm. In some cases, the inner diameter 148 of the inner lumen of the obturator 119 may comprise a diameter of at most about 0.3 mm, about 0.4 mm, about 0.5 mm, about 0.6 mm, about 0.7 mm, about 0.8 mm, about 0.9 mm, about 1 mm, about 1.2 mm, or about 1.4 mm.

The obturator 119 may be configured to assist the insertion of the elongated body 116 into the patient/subject receiving the electrode lead implant by providing structural rigidity. The obturator elongated body 117 may comprise a region 151 that protrudes a distance out from the elongated body 116, as seen in FIG. 1D. The region 151 that protrudes a distance out from the elongated body 116 may comprise a blunt protrusion. The region 151 that protrudes a distance out from the elongated body 116 may comprise a protrusion with angle parallel to that of the angle facet 145 of the elongated body 116, described elsewhere herein. The region 151 may comprise a tapered tip. The tapered tip may comprise a fine, bullet tip configured to penetrate regions with high tissue density.

The distance of the protrusion 151 that may extend beyond the end of the elongated body 116 may extend by a distance of about 0.2 mm to about 3 mm. For example, the distance of the protrusion 151 that may extend beyond the end of the elongated body 116 may extend by a distance of about 0.4 mm to about 3 mm, about 1 mm to about 3 mm, or about 1.4 mm to about 3 mm. The distance of the protrusion 151 that may extend beyond the end of the elongated body 116 may extend by a distance of about 0.2 mm, about 0.4 mm, about 0.6 mm, about 0.8 mm, about 1 mm, about 1.2 mm, about 1.4 mm, about 1.6 mm, about 1.8 mm, about 2 mm, about 2.5 mm, or about 3 mm. In some instances, the distance of the protrusion 151 that may extend beyond the end of the elongated body 116 may extend by a distance of at least about 0.2 mm, about 0.4 mm, about 0.6 mm, about 0.8 mm, about 1 mm, about 1.2 mm, about 1.4 mm, about 1.6 mm, about 1.8 mm, about 2 mm, or about 2.5 mm. In some instances, the distance of the protrusion 151 that may extend beyond the end of the elongated body 116 may extend by a distance of at most about 0.4 mm, about 0.6 mm, about 0.8 mm, about 1 mm, about 1.2 mm, about 1.4 mm, about 1.6 mm, about 1.8 mm, about 2 mm, about 2.5 mm, or about 3 mm.

The obturator handle 104 may mechanically couple to the sheath handle 106, as shown in FIG. 1A and FIG. 1E. The mechanical coupling between the obturator handle 104 and the sheath handle 106 may comprise a hook and latch, quick release, or any combination thereof. The obturator handle 104 may comprise a coupling receptacle 105, configured to receive and fasten to the coupling mechanism of the needle handle 129, as seen in FIG. 1E. The coupling receptacle 105 may comprise a coupling feature 121 configured to interface with the needle handle 129 when the needle body 102 is inserted into the inner lumen of obturator elongated body 117. For example, the coupling feature 121 may be configured to slide within a track on the needle handle 129. The coupling feature 121 may interface with the track of the needle handle 129 at a location of the track with an interference fit, thereby providing an interference fit based mechanical fastening between the needle 115 and the obturator 119.

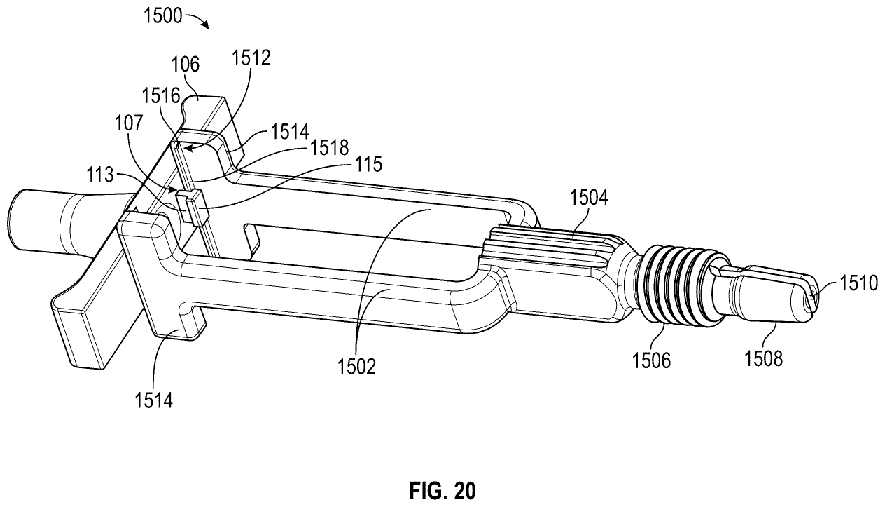

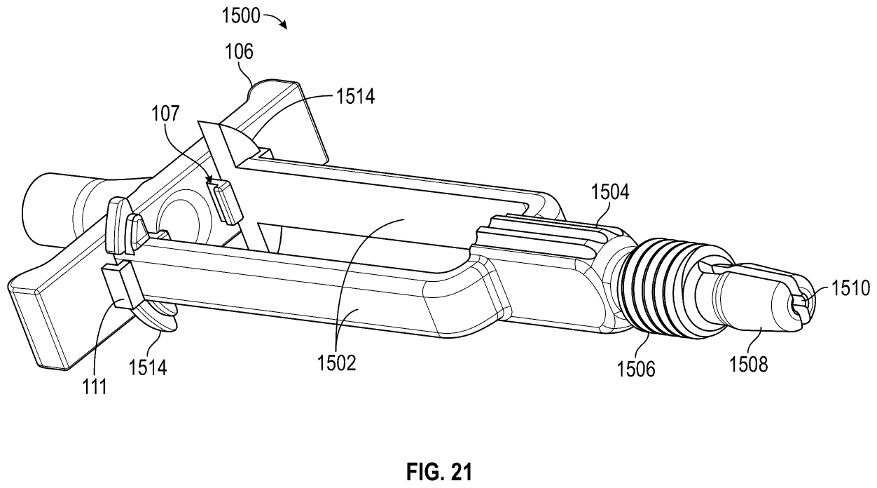

The obturator handle 104 may mechanically couple to the sheath handle 106, as shown in FIG. 1H. The mechanical coupling between the obturator handle 104 and the sheath handle 106 may comprise a hook and latch, quick release, or any combination thereof. For example, the obturator handle 104 may comprise an overhang 2606. The introducer handle 106 may comprise receiving tabs 2608 configured to receive and fasten to the overhang 2606 of the obturator handle 104. The receiving tabs 2608 may be configured to be coupled to receive and couple a LPG, as shown in FIGS. 20-21.

The obturator 119 may comprise an obturator elongated body 117 having stiffener tube 2602 and an obturator handle 104 at an end of the obturator elongated body. The stiffener tube 2602 may be comprised of a plastic (e.g., ABS), metal, or any combination thereof. The metal may comprise stainless steel, aluminum, titanium, or any combination thereof. The obturator stiffener tube may be comprised of a stainless-steel, aluminum, titanium, or any combination thereof material while the obturator handle 104 may comprise a plastic. The obturator handle comprises a high viscosity polyamide. For example, the obturator handle comprises Vestamid.

The obturator 119 may comprise an obturator elongated body 117 having a lumen and an obturator handle 104 at an end of the obturator elongated body, as shown in FIG. 1F. The obturator elongated body 117 may be comprised of a plastic (e.g., ABS), metal, or any combination thereof.

The needle 115 may comprise a needle elongated body 102, a needle handle 129 at one end of the needle elongate shaft, and a needle tip 118 at the other end of the needle elongate shaft. The needle elongated body 102 may be configured to fit into the obturator inner lumen, designated by an inner diameter 148 of the obturator inner lumen. The needle elongated body may comprise an outer diameter 138, as seen in FIG. 1D. The fit may comprise a slip fit between the inner diameter 148 of the obturator inner lumen and the outer diameter 138 of the needle elongated body. The needle 115 may be comprised of stainless steel, aluminum, titanium, or any combination thereof. The needle 115 may be hollow or maybe be partially hollow and/or partially solid. The needle 115 may be comprised of a rigid non-deformable plastic and/or polymer.

The needle elongated body outer diameter 138 may comprise a diameter of about 0.1 mm to about 3 mm. For example, the needle elongated body outer diameter 138 may comprise a diameter of about 0.3 mm to about 3 mm, about 0.5 mm to about 3 mm, or about 1 mm to about 3 mm. The needle elongated body outer diameter 138 may comprise a diameter of about 0.1 mm, about 0.2 mm, about 0.3 mm, about 0.4 mm, about 0.5 mm, about 0.6 mm, about 0.8 mm, about 1 mm, about 1.5 mm, about 2 mm, about 2.5 mm, or about 3 mm. In some instances, the needle elongated body outer diameter 138 may comprise a diameter of at least about 0.1 mm, about 0.2 mm, about 0.3 mm, about 0.4 mm, about 0.5 mm, about 0.6 mm, about 0.8 mm, about 1 mm, about 1.5 mm, about 2 mm, or about 2.5 mm. In some instances, the needle elongated body outer diameter 138 may comprise a diameter of at most about 0.2 mm, about 0.3 mm, about 0.4 mm, about 0.5 mm, about 0.6 mm, about 0.8 mm, about 1 mm, about 1.5 mm, about 2 mm, about 2.5 mm, or about 3 mm.

The needle elongated body outer diameter 138 may comprise a diameter of about 12 American Wire Gauge (AWG) to about 26 AWG. For example, the needle elongated body outer diameter 138 may comprise a diameter of about 12 AWG to about 14 AWG, about 12 AWG to about 16 AWG, about 12 AWG to about 18 AWG, about 12 AWG to about 20 AWG, about 12 AWG to about 22 AWG, about 12 AWG to about 24 AWG, about 12 AWG to about 26 AWG, about 14 AWG to about 16 AWG, about 14 AWG to about 18 AWG, about 14 AWG to about 20 AWG, about 14 AWG to about 22 AWG, about 14 AWG to about 24 AWG, about 14 AWG to about 26 AWG, about 16 AWG to about 18 AWG, about 16 AWG to about 20 AWG, about 16 AWG to about 22 AWG, about 16 AWG to about 24 AWG, about 16 AWG to about 26 AWG, about 18 AWG to about 20 AWG, about 18 AWG to about 22 AWG, about 18 AWG to about 24 AWG, about 18 AWG to about 26 AWG, about 20 AWG to about 22 AWG, about 20 AWG to about 24 AWG, about 20 AWG to about 26 AWG, about 22 AWG to about 24 AWG, about 22 AWG to about 26 AWG, or about 24 AWG to about 26 AWG. The needle elongated body outer diameter 138 may comprise a diameter of about 12 AWG, about 14 AWG, about 16 AWG, about 18 AWG, about 20 AWG, about 22 AWG, about 24 AWG, or about 26 AWG. In some instances, the needle elongated body outer diameter 138 may comprise a diameter of at least about 12 AWG, about 14 AWG, about 16 AWG, about 18 AWG, about 20 AWG, about 22 AWG, or about 24 AWG. In some instances, the needle elongated body outer diameter 138 may comprise a diameter of at most about 14 AWG, about 16 AWG, about 18 AWG, about 20 AWG, about 22 AWG, about 24 AWG, or about 26 AWG.

The needle handle 129 may comprise a coupling feature 130 configured to couple to the coupling feature 121 of the obturator, described elsewhere herein. The needle handle coupling feature 130 may comprise a path or a slot feature, whereby the obturator coupling feature 121, upon inserting the needle 115 into the inner lumen of the obturator, may travel within and apply a tension and/or holding force when rotated around the central axis of the needle 115 and obturator 119. The needle handle may be rotated by rotating one or more flanges 131 of the needle handle 129. The rotation may be accomplished by the user, medical personnel, surgeon or any combination thereof personnel. In some embodiments, the rotation may be accomplished by a motor.

The needle tip 118 may configured to protrude beyond an end of the obturator lumen. The needle tip 118 may protrude 136 at least 1 mm beyond the end of the obturator lumen. The needle tip may be configured to protrude by a movement of the needle handle 129. The needle tip 118 may be configured to be retractable into the obturator lumen.

The needle tip 118 may have an angle 146 ranging from about 15 degrees to about 45 degrees from the needle elongated body. In some embodiments, the needle tip 118 may have an angle 146 ranging from about 25 degrees to about 50 degrees, or about 35 degrees to about 50 degrees. The needle tip 118 may have an angle 146 ranging from about 15 degrees to about 45 degrees from the needle elongated body about 15 degrees, about 25 degrees, about 35 degrees, about 40 degrees, about 45 degrees, about 50 degrees, or about 1 degree. The needle tip 118 may have an angle 146 ranging from about 15 degrees to about 45 degrees from the needle elongated body at least about 15 degrees, about 25 degrees, about 35 degrees, about 40 degrees, about 45 degrees, or about 50 degrees. The needle tip 118 may have an angle 146 ranging from about 15 degrees to about 45 degrees from the needle elongated body at most about 25 degrees, about 35 degrees, about 40 degrees, about 45 degrees, about 50 degrees, or about 1 degree. The needle tip 118 angle 146 may be configured to advance the needle tip through a soft tissue.

The electrode lead introducer 100 may comprise one or more electrodes (120, 110), as seen in FIGS. 1A, 1B, 1E, and 1F, configured to provide electrical stimulation and/or to measure electrical signals adjacent to a target tissue in patient's pelvic region. For example, the target tissue may comprise a pudendal nerve, a sacral nerve, another nerve, or a nerve branch, or a combination thereof. The electrodes may be configured to assist the user, medical personnel, and/or surgeon, in navigating to target region of the patient's pelvic region. The target region of the patient's pelvic region may comprise portions of the patient's pudendal, sacral, or any combination thereof nerves or any branches thereof. In some cases, the electrodes may be adjacent to one or more insulators (108, 123). The one or more insulators (108, 123) may be configured to not conduct and/or sense electrical current.

The electrode lead introducer 100 may comprise two sets of one or more electrodes (120, 110) and two sets of one or more insulators (108, 123) whereby each set of electrodes and/or insulators are located at opposite ends of the electrode lead introducer, as seen in FIGS. 1A-1B. Designating for purposes of example only, the needle tip 118 as the proximal end of the electrode lead introducer 100, the first set of one or more electrodes 120 and/or the first set of one or more insulators 108 may be located at a proximal end of the electrode lead introducer, as shown in FIG. 1B. Designating for purposes of example only, the sheath handle 106 as the distal end of the electrode lead introducer 100, the second set of one or more electrodes 110 and/or the second set of one or more insulators 123 may be located at a distal end of the electrode lead introducer. The one or more electrodes located at the proximal end of the electrode lead introducer 120 may be in electrical communication with the one or more electrodes located at the distal end of the electrode lead introducer 110. The proximal end one or more electrodes 120 may be configured to detect and/or provide electrical signals to a target tissue in patient's pelvic region. For example, the target tissue in the patient's pelvic region comprises a pudendal nerve, a sacral nerve, or a combination thereof. The target tissue in the patient's pelvic region may comprise a trunk of a nerve or a branch of the nerve or a combination thereof. The one or more electrodes (120, 110) at the distal and/or proximal region of the electrode lead introducer may comprise at least one, at least two, at least three, at least four, at least five, or at least six electrodes. The one or more electrodes (120, 110) at the distal and/or proximal region of the electrode lead introducer may comprise at most one, at most two, at most three, at most four, at most five, or at most six electrodes. The distal one or more electrodes may be configured to couple to terminations of a hook type probe, where the hook type probe may provide an electrical stimulation signal and/or detect an electrical signal via the one or more electrodes at the distal end 120. The hook type probe may be in electrical communication with the one or more distal and/or proximal electrodes.

The one or more electrodes located at the distal 110 and proximal 120 end and/or the one or more insulators located at the distal 120 and proximal 108 end of the electrode lead introducer may comprise electrodes and insulators of varying length, as can be seen in FIG. 1B. The one or more proximal electrodes 120, may comprise a length 128. The length 128 of the proximal one or more electrodes 120 may comprise about 0.8 mm to about 2 mm. For example, the length 128 of the proximal one or more electrodes 120 may comprise about 1 mm to about 2 mm, or about 1.3 mm to about 2 mm. The length 128 of the proximal one or more electrodes 120 may comprise about 0.8 mm, about 0.9 mm, about 1 mm, about 1.1 mm, about 1.2 mm, about 1.3 mm, about 1.4 mm, about 1.5 mm, or about 2 mm. In some cases, the length 128 of the proximal one or more electrodes 120 may comprise at least about 0.8 mm, about 0.9 mm, about 1 mm, about 1.1 mm, about 1.2 mm, about 1.3 mm, about 1.4 mm, or about 1.5 mm. In some cases, the length 128 of the proximal one or more electrodes 120 may comprise at most about 0.9 mm, about 1 mm, about 1.1 mm, about 1.2 mm, about 1.3 mm, about 1.4 mm, about 1.5 mm, or about 2 mm.

The length 126 of the proximal one or more insulators 108 may comprise about 5 mm to about 7 mm. For example, the length 126 of the proximal one or more insulators 108 may comprise about 5.5 mm to about 7 mm, or about 6 mm to about 7 mm. The length 126 of the proximal one or more insulators 108 may comprise about 5 mm, about 5.1 mm, about 5.2 mm, about 5.3 mm, about 5.4 mm, about 5.5 mm, about 5.8 mm, about 6 mm, about 6.5 mm, or about 7 mm. In some cases, the length 126 of the proximal one or more insulators 108 may comprise at least about 5 mm, about 5.1 mm, about 5.2 mm, about 5.3 mm, about 5.4 mm, about 5.5 mm, about 5.8 mm, about 6 mm, or about 6.5 mm. In some cases, the length 126 of the proximal one or more insulators 108 may comprise at most about 5.1 mm, about 5.2 mm, about 5.3 mm, about 5.4 mm, about 5.5 mm, about 5.8 mm, about 6 mm, about 6.5 mm, or about 7 mm.

The length 124 of the distal one or more electrodes 110 may comprise a length of about 2.5 mm to about 4 mm. For example, the length 124 of the distal one or more electrodes 110 may comprise a length of about 2.6 mm to about 4 mm, or 3 mm to about 4 mm. The length 124 of the distal one or more electrodes 110 may comprise a length of about 2.5 mm, about 2.6 mm, about 2.7 mm, about 2.8 mm, about 2.9 mm, about 3 mm, about 3.1 mm, about 3.5 mm, or about 4 mm. In some cases, the length 124 of the distal one or more electrodes 110 may comprise a length of at least about 2.5 mm, about 2.6 mm, about 2.7 mm, about 2.8 mm, about 2.9 mm, about 3 mm, about 3.1 mm, or about 3.5 mm. In some cases, the length 124 of the distal one or more electrodes 110 may comprise a length of at most about 2.6 mm, about 2.7 mm, about 2.8 mm, about 2.9 mm, about 3 mm, about 3.1 mm, about 3.5 mm, or about 4 mm. The first electrode of the one or more distal electrodes 108 may be spaced a distance of at least about 1.5 mm from the distal most portion of the elongated body 116.

The length 122 of the distal one or more insulators 123 may comprise a length of about 1.2 mm to about 3 mm. For example, the length 122 of the distal one or more insulators 123 may comprise a length of about 1.5 mm to about 3 mm, or about 1.8 mm to about 3 mm. The length 122 of the distal one or more insulators 123 may comprise a length of about 1.2 mm, about 1.3 mm, about 1.4 mm, about 1.5 mm, about 1.6 mm, about 1.7 mm, about 1.8 mm, about 1.9 mm, about 2 mm, about 2.5 mm, or about 3 mm. In some cases, the length 122 of the distal one or more insulators 123 may comprise a length of at least about 1.2 mm, about 1.3 mm, about 1.4 mm, about 1.5 mm, about 1.6 mm, about 1.7 mm, about 1.8 mm, about 1.9 mm, about 2 mm, or about 2.5 mm. In some cases, the length 122 of the distal one or more insulators 123 may comprise a length of at most about 1.3 mm, about 1.4 mm, about 1.5 mm, about 1.6 mm, about 1.7 mm, about 1.8 mm, about 1.9 mm, about 2 mm, about 2.5 mm, or about 3 mm.

Usually, the electrodes may be manufactured by various methods. The electrode may comprise a flexible printed circuit. The electrode may be wrapped around the outer surface of the sheath elongate shaft. In some embodiments, the electrode may be bonded to the outer surface of the sheath elongate shaft. The manufacturing method chosen may facilitate a large-scale manufacturing of the electrode in bulk. In some embodiments, the manufacturing method chosen may facilitate accurate manufacturing of the electrode with low tolerances.

The electrodes may be designed to deliver various amounts of voltage, current, and/or power. The electrodes may be designed to deliver a voltage of about 10V per electrode. The electrodes may be designed to deliver a voltage of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 V per electrode. In some embodiments, the electrodes may be designed to deliver a voltage of at most about 5, 10, 15, 20, 25, 30, 35, 40, 45, or 50 V per electrode. The electrodes may be designed to deliver a voltage of about 1 to about 50 V per electrode, about 1 to about 40 V per electrode, about 1 to about 30 V per electrode, or about 1 to about 20 V per electrode. The electrodes may be designed to deliver a current of about 10 mA per electrode. In some embodiments, the electrodes may be designed to deliver a current of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mA per electrode. The electrodes may be designed to deliver a current of at most about 5, 10, 15, 20, 25, 30, 35, 40, 45, or 50 mA per electrode. The electrodes may be designed to deliver a current of about 1 to about 50 mA per electrode, about 1 to about 40 mA per electrode, about 1 to about 30 mA per electrode, or about 1 to about 20 mA per electrode. The electrodes may be designed to deliver a power (VA) of about 0.1 W per electrode. In some embodiments, the electrodes may be designed to deliver a power (VA) of at least about 0.01, 0.05, 0.1, 0.5, 1, 2, 3, 4, or 5 W per electrode. In some embodiments, the electrodes may be designed to deliver a power (VA) of at most about 0.05, 0.1, 0.5, 1, 2, 3, 4, 5, 6, 7, 8, 9, or 10 W per electrode. The electrodes may be designed to deliver a power (VA) of about 0.01 to about 10 W per electrode, about 0.01 to about 5 W per electrode, or about 0.01 to about 1 W per electrode.

Provided herein are devices for placing an electrode lead to at a target tissue in a patient's pelvic region, the device having one or more of the following instruments: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. The stiffness of the sheath and the needle together may be sufficient to allow secure, accurate placement of the electrode lead at a target tissue in a patient's pelvic region. For example, the target tissue may comprise a pudendal nerve. In some embodiments, the stiffness of the sheath may be sufficient to allow secure, accurate placement of the electrode lead at a target tissue in a patient's pelvic region. For example, the target tissue may comprise a pudendal nerve.

Described herein are devices for placing an electrode lead at a target tissue in a pelvic region of a patient, the device may include one or more of the following features: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. The needle may be removable from the introducer sheath. The insertable length of the sheath may be about 10 cm to about 20 cm. The introducer sheath may have an outer diameter of about 1 mm to about 5 mm. The introducer sheath may have an inner diameter of about 1 mm to about 3 mm. The introducer sheath may have an inner diameter sufficient for the obturator and the needle to pass through. The inner diameter of the needle shaft may be sufficient for the guidewire to pass through. The needle tip may be blunt and may have a lumen. The needle tip may extend about 1 mm to about 5 mm beyond the end of the sheath elongate shaft. The stiffness of the sheath and the needle together may be sufficient to allow secure, accurate placement of the electrode lead at a target tissue in a patient's pelvic region. For example, the target tissue may comprise a pudendal nerve. In some embodiments, the stiffness of the sheath may be sufficient to allow secure, accurate placement of the electrode lead at a target tissue in a patient's pelvic region. For example, the target tissue may comprise a pudendal nerve. In some embodiments, the stiffness of the sheath is higher than stiffness of sheath for sacral nerve lead placement.

Described herein are devices for placing an electrode lead to at a target tissue in a patient's pelvic region, the device may include one or more of the following features: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. The needle may be removable from the introducer sheath. The insertable length of the sheath may be about 10 cm to about 20 cm. The introducer sheath may have an outer diameter of about 1 mm to about 5 mm. For example, the introducer sheath may have an inner diameter of about 1 mm to about 3 mm. The introducer sheath may have an inner diameter sufficient for the obturator and the needle to pass through. The inner diameter of the needle shaft may be sufficient for the guidewire to pass through. The needle tip may be blunt and may have a lumen. The needle tip may extend about 1 mm to about 5 mm beyond the end of the sheath elongate shaft. The sheath may comprise a plurality of electrically isolated electrodes. The plurality of electrodes may form about a plurality of wide bands around the sheath with a gap between electrodes and the first band about at least 1 mm from the end of the sheath elongate shaft. The stiffness of the sheath and the needle together may be sufficient to allow secure, accurate placement of the electrode lead at a target tissue in a patient's pelvic region. For example, the target tissue may comprise a pudendal nerve. In some embodiments, the stiffness of the sheath may be sufficient to allow secure, accurate placement of the electrode lead at a target tissue in a patient's pelvic region. For example, the target tissue may comprise a pudendal nerve. The stiffness of the sheath for pudendal nerve lead placement may be higher than stiffness of sheath for sacral nerve lead placement. The electrodes may be configured to deliver a voltage of about 5V to about 15V per electrode. The electrodes may be configured to deliver a current of about 5 mA to about 15 mA per electrode. The electrodes may be configured to deliver a power of about 0.05 W to about 0.5 W per electrode.

Provided herein are devices for placing an electrode lead to a target tissue in a patient's pelvic region, the device may include one or more of the following instruments: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. The needle may be removable from the introducer sheath. The insertable length of the sheath may be about 15 cm. The introducer sheath may have a maximum outer diameter of about 5 mm. The introducer sheath may have an inner diameter of about 1 mm to about 2 mm. The introducer sheath may have an inner diameter sufficient for the obturator and the needle to pass through. The inner diameter of the needle shaft may be sufficient for the guidewire to pass through. The needle tip may be blunt and may have a lumen. The needle tip may extend a maximum of 3 mm beyond the end of the sheath elongate shaft. The sheath may comprise four electrically isolated electrodes forming about 1.5 mm wide bands around the sheath with a gap between electrodes of about 5 mm and the first band about 1.5 mm from the end of the sheath elongate shaft. The stiffness of the sheath and the needle together may be sufficient to allow secure, accurate placement of the electrode lead at a target tissue in a patient's pelvic region. For example, the target tissue may comprise a pudendal nerve. In some embodiments, the stiffness of the sheath may be sufficient to allow secure, accurate placement of the electrode lead at a target tissue in a patient's pelvic region. For example, the target tissue in the patient's pelvic region may comprise a pudendal nerve. The stiffness of the sheath for pudendal nerve lead placement may be higher than stiffness of sheath for sacral nerve lead placement. The electrodes may be configured to deliver a voltage of about 10V per electrode. The electrodes may be configured to deliver a current of about 10 mA per electrode. The electrodes may be configured to deliver a power of about 0.1 W per electrode.

Described herein are devices for placing an electrode lead at a target tissue in a patient's pelvic region, the device may include one or more of the following instruments: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. The needle tip may be blunt and has a lumen. The needle tip may be configured to protrude between about 1 mm to 5 mm beyond the end of the obturator lumen. The needle tip may be configured to be retractable into the obturator lumen. The needle tip angle may be configured to advance the needle tip through tissue. The angle of the proximal end of the sheath elongate shaft may allow for advancing the device with little to no damage to surrounding tissue. The electrode may be wrapped around the outer surface of the sheath elongate shaft.

Provided herein are devices for placing an electrode lead at a target tissue in a patient's pelvic region, the device may include one or more of the following instruments: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. The needle tip may be blunt and has a lumen. The needle tip may be configured to protrude between about 1 mm to 5 mm beyond the end of the obturator lumen. The needle tip may be configured to be retractable into the obturator lumen. The needle tip angle may be configured to advance the needle tip through tissue. The angle of the proximal end of the sheath elongate shaft may allow for advancing the device with little to no damage to surrounding tissue. The electrode may be wrapped around the outer surface of the sheath elongate shaft.

Described herein are devices for placing an electrode lead to at a target tissue in a patient's pelvic region, the device may include one or more of the following instruments: an introducer sheath comprising a sheath elongate shaft having a lumen and a sheath handle at a distal end of the sheath shaft, wherein the introducer sheath is configured to carry an electrode on an outer surface of the sheath elongate shaft; an obturator comprising an obturator elongate shaft having a lumen and an obturator handle at a distal end of the obturator elongate shaft, wherein the obturator elongate shaft is configured to fit inside the sheath lumen; and a needle comprising a needle elongate shaft having a lumen, a needle handle at a distal end of the needle elongate shaft, and a needle tip at the proximal end of the needle elongate shaft, wherein the needle elongate shaft is configured to fit inside the obturator lumen, and wherein the lumen of the needle elongate shaft is configured to allow the electrode lead to pass through; wherein the introducer sheath has a stiffness sufficient to guide the placement of the electrode lead at the target site. The needle tip may be blunt and may have a lumen. The needle tip may be configured to protrude between about 1 mm to 5 mm beyond the end of the obturator lumen. The needle may be removable from the introducer sheath. The insertable length of the sheath may be about 10 cm to about 20 cm. The introducer sheath may have an outer diameter of about 1 mm to about 5 mm. The introducer sheath may have an inner diameter of about 1 mm to about 3 mm. The introducer sheath may have an inner diameter sufficient for the obturator and the needle to pass through. The inner diameter of the needle shaft may be sufficient for the guidewire to pass through. The needle tip may be blunt and may have a lumen. The needle tip may extend about 1 mm to about 5 mm beyond the end of the sheath elongate shaft. The sheath may comprise a plurality of electrically isolated electrodes. The plurality of electrodes may form about a plurality of wide bands around the sheath with a gap between electrodes and the first band about at least 1 mm from the end of the sheath elongate shaft. The needle tip may be configured to be retractable into the obturator lumen. The electrode may be wrapped around the outer surface of the sheath elongate shaft. The needle tip angle may be configured to advance the needle tip through tissue. The angle of the proximal end of the sheath elongate shaft may allow for advancing the device with little to no damage to surrounding tissue. The obturator may have a diameter of about 1 mm to about 4 mm. The introducer sheath may have a diameter of about 1 mm to about 5 mm. The introducer sheath and the needle may have a combined Young's modulus sufficient to allow for the device to penetrate a deep surgical plane in an individual. The deep surgical plane may comprise a surgical plane of muscle, fat, or any combination thereof. The introducer sheath may have a Young's modulus of about 10 mega pascal (MPa) to about 10,000 MPa. The introducer sheath and needle may have a combined Young's modulus sufficient to allow a user to place the lead adjacent to a target tissue in a patient's pelvic region. For example, the target tissue in the patient's pelvic region may comprise a pudendal nerve. The stiffness of the sheath and the needle together may be sufficient to allow secure, accurate placement of the electrode lead at a target tissue in the patient's pelvic region. For example, the target tissue may comprise a pudendal nerve. In some embodiments, the stiffness of the sheath may be sufficient to allow secure, accurate placement of the electrode lead at a target tissue in the patient's pelvic region. For example, the target tissue may comprise a pudendal nerve. The stiffness of the sheath for pudendal nerve lead placement may be higher than stiffness of sheath for sacral nerve lead placement. The electrodes may be configured to deliver a voltage of about 5V to about 15V per electrode. The electrodes may be configured to deliver a current of about 5 mA to about 15 mA per electrode. The electrodes may be configured to deliver a power of about 0.05 W to about 0.5 W per electrode. The needle tip may be configured to be retractable into the obturator lumen. The needle tip angle may be configured to advance the needle tip through tissue. The angle of the proximal end of the sheath elongate shaft may allow for advancing the device with little to no damage to surrounding tissue. The electrode may be wrapped around the outer surface of the sheath elongate shaft.

### Electrode Lead Placement

Described herein are methods, devices, systems, and/or kits for placing at least one electrode lead at a target tissue, for example in the pelvic region. The target tissue may comprise a pudendal nerve to treat incontinence. In some cases, the target tissue may comprise a target tissue to receive electrical stimulation for sexual dysfunction. In some instances, the target tissue may comprise a target tissue to receive electrical stimulation for pain treatment and/or management. The electrode lead may be implanted at one or more target nerves in the pelvic region. For example, the target nerve(s) may comprise one or more of the pudendal nerve, tibial nerve, peroneal nerve, sacral nerve, cauda equina, pelvic parasympathetic nerves, lumbar sympathetic nerves, and/or nerve branches leading to and from such nerves. Although certain devices, systems, methods, and kits are described herein with respect to the pelvic region to treat and/or manage pelvic conditions, the methods and devices can be used in other regions of the body or to treat other conditions as described elsewhere herein.

In some embodiments, the devices, systems, methods, and/or kits provided herein may be specific to neuromodulation surgical techniques associated with a pelvic nerve, e.g., one or more of a pudendal nerve, sacral nerve, tibial nerve, peroneal nerve, sacral nerve, cauda equina, pelvic parasympathetic nerves, lumbar sympathetic nerves, and/or nerve branches leading to and from such nerves, etc. In some embodiments, the devices, systems, methods, and/or kits provided herein may be specific to training of a neuromodulation surgical technique associated with one or more nerves associated with incontinence, including but not limited to urinary incontinence (such as urge, stress or mixed urinary incontinence), overactive bladder, fecal incontinence, etc. Some embodiments are particular well suited for training of tissue (e.g., nerve) targeting in the pelvic region because, for example, pelvic nerves may have an increased level of difficulty for surgeons to locate and place a device, such as an electrode lead, adjacent to, because of the complicated anatomical structures and the tortuous pathways followed by the nerves.

The methods, devices, systems, and/or kits provided herein may be used to place at least one electrode lead on a nerve that serve one or more muscles controlling urination to treat urinary incontinence. In some cases, the methods, devices, systems, and/or kits provided herein may be used to place at least one electrode lead on a nerve that serve one or more muscles used for urination to treat fecal incontinence. In some cases, methods, devices, systems, and/or kits provided herein may be used to bilaterally place at least one electrode lead on each side of the body at a nerve that serves one or more muscles controlling or used for urination. In some embodiments, the devices, systems, methods, and/or kits provided herein may be used for neuromodulation surgical techniques associated with one or more of a tibial nerve, peroneal nerve, sacral nerve, cauda equina, pelvic parasympathetic nerves, lumbar sympathetic nerves, and/or nerve branches leading to and from such nerves influencing bowel or bladder function. For example, bilateral stimulation of the pudendal nerve may allow for better control and/or effectiveness in treating urinary or fecal incontinence than unilateral stimulation. In some embodiments, bilateral stimulation may be beneficial when one lead is implanted at one target nerve, e.g., a pudendal nerve, and the other lead is implanted at another target nerve, e.g., a sacral nerve. Often, accessing the pudendal nerve and placing an electrode or an electrode lead with minimal injury to the surrounding tissues may be difficult by the anatomical structure near the pudendal nerve. The introducer may allow for access of the pudendal nerve by one or more anatomical paths with little damage to surrounding tissues. In some cases, the introducer may access the pudendal nerve by an ischiorectal approach, where the introducer directed to penetrate or pass close to the sacrotuberous ligament and place the lead to the pudendal nerve trunk at a target location proximal to Alcock's canal. In some cases, the introducer may access the pudendal nerve by a low gluteal approach, where the introducer is directed to pass in the space between sacrotuberous ligament and sacrospinous ligament and to pass anteriorly in the ischiorectal fossa below the pelvic floor to place the lead on the anterior branches of the pudendal nerve to stimulate the pudendal nerve and the dorsal genital nerve.

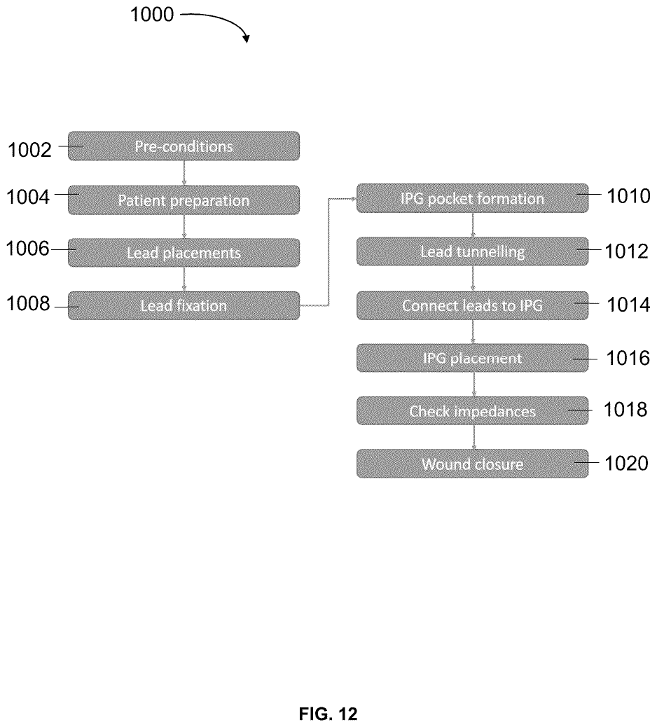

Provided herein are methods, devices, and systems for implanting at least one electrode lead and an implantable pulse generator (IPG) in an individual. Usually, the implantation procedure may involve one or more of steps of pre-condition, patient preparation, nerve localization, placement of one or more leads, fixation of the leads, IPG pocket formation, lead tunneling, connecting the one or more leads to IPG, IPG placement, check of impedances, and tissue closure. FIG. 12 shows a flowchart 1000 of the steps in for performing electrode lead and implantable pulse generator (IPG) implantation. In some embodiments, the implantation procedure involves sequential steps of pre-condition 1002, patient preparation 1004, placement of one or more leads 1006, fixation of the leads 1008, IPG pocket formation 1010, lead tunneling 1012, connecting the one or more leads to IPG 1014, IPG placement 1016, check of impedances 1018, and wound closure 1020. The steps may be performed by a healthcare professional or a surgeon in an individual to treat incontinence. In some embodiments, the steps may be performed by or controlled by robotic systems and/or facilitated using augmented reality.

Provided herein are methods, devices, and systems to allow for accessing the pudendal nerve by one or more anatomical paths. First, the patient may be positioned in a prone position so as to elevate the buttocks to enable lead insertion by posterior or gluteal approaches. Often, the buttocks may be marked using surface landmarks, including but not limited to greater trochanter and ischial tuberosity, to locate the surface position of the ischial spine. Using these landmarks and plain radiological imaging (C-arm image intensifier of equivalent), marking needles and/or needle electrodes may be inserted to locate the pudendal nerve at each target location. In some cases, the target location may comprise distal/pelvic floor via low gluteal approach and proximal trunk of the pudendal nerve via gluteal approach. The nerves may be additionally, or alternatively, located by intra-operative electrophysiology (EMG responses). The nerves may be additionally, or alternatively, located by visual motor responses. The nerves may be additionally, or alternatively, located by urethral pressure measurements. Once the nerves are located, the introducer may be used to follow the path of the needles to each target location. The introducers at the location may be advanced and fine alterations may be made to their position such that stimulation via a defined proportion of the total number of electrodes leads to a pelvic floor EMG, urethral sphincter, or anal EMG response. The fine alterations may be made to position of the introducer and the electrodes such that stimulation by at least one of the electrodes leads to a pudendal EMG response. In some cases, the fine alterations may be made to position of the introducer and the electrodes such that stimulation by the majority or all of the electrodes leads to a response. Once the introducers are adequately positioned, the obturator may be withdrawn and replaced by the electrode leads using the markings provided to accurately align the lead electrodes with the rings on the introducer. The introducers may be carefully removed (under image intensification) so as not to disturb lead positioning. A small skin incision may be made to facilitate access to the leads, which may be then fixed in position e.g., by using the fixation devices (threaded onto the lead) and standard non-absorbable monofilament sutures to local fascia. The leads may be then tunneled to the future IPG site.

An electrode lead may be placed onto a target area of the target nerve for treating incontinence using the introducer device described herein. An electrode lead may be placed onto a target area of the target nerve for treating incontinence using a guidewire and/or sheath. For example, the target nerve(s) may comprise one or more of the pudendal nerve, tibial nerve, peroneal nerve, sacral nerve, cauda equina, pelvic parasympathetic nerves, lumbar sympathetic nerves, and/or nerve branches leading to and from such nerves. The electrode needle may comprise a Chiba needle. The lead or guidewire introducer may comprise a metal obturator or stiffening wire and an insulating plastic sheath. The lead or guidewire introducer may be modified to allow easier access to the pudendal nerve. The lead or guidewire introducer may be configured to perforate a ligament. The lead or guidewire introducer may be configured to allow access close to the sacrotuberous ligament. The sheath of the introducer to access the pudendal nerve may have a higher stiffness than a sheath of an introducer typically used to access the sacral nerve.

The lead may comprise a sensor that can obtain neurophysiological recordings from a nerve, such as the pudendal nerve. In some embodiments, the methods described herein may comprise placing a sensor on the pudendal nerve to obtain electrical signal from the pudendal nerve. The obtained electrical signal may be used to determine the level of neurostimulation of the pudendal nerve to prevent an incontinence episode. In some embodiments, individuals may control stimulation by performing a pelvic squeeze, wherein a sensor receiving a threshold EMG signal due to the pelvic squeeze may activate electrical stimulation.

### Pudendal Nerve

The pudendal nerve is a major nerve in the pelvic region. Usually, the pudendal nerve may run through the pelvic floor muscles that support organs and ends at external genitalia. Often, the pudendal nerve may send motor and sensation information from the genital area. The pudendal nerve may be crucial for sensation and function in the pelvic region. The pudendal nerve may be a part of the peripheral nervous system.

Typically, the pudendal nerve is found bilaterally, one for each side of the body, on the left and the right. The pudendal nerve may usually arise from the sacral plexus in the lowest part of the spine. The sacral plexus comprises a bundle of nerves located on the back of the pelvis. The sacral plexus may comprise a complex network of nerves that give and receive feedback on movement and sensation to the thighs, lower legs, feet, and part of the pelvis. Usually, the pudendal nerve connects to the S2 to S4 sacral spinal nerve roots in the sacral plexus and runs through the pelvis and gluteal region at the upper end of the femur. Often, the pudendal nerve passes through the greater sciatic foramen, exits the gluteal region through the lesser sciatic foramen, and travels alongside the pudendal artery and vein into the pudendal canal, also referred herein as the Alcock's canal, a narrow tunnel-like opening in the pelvis. The pudendal nerve may divide into smaller nerve branches after entering the pudendal canal. The pudendal nerve may branch into the inferior rectal nerve, perineal nerve, and dorsal genital nerve. The pudendal nerve may run on the inner side of and medial to the ischial bone. The pudendal nerve may be difficult to access surgically because it runs in three different planes. The inferior rectal nerve may control the anal sphincter and sends sensory and motor information to the anal sphincter and anal canal. The pudendal nerve may play a role in reflex control of bladder contraction and emptying. The perineal nerve may control the pelvic floor muscles and the urethral sphincter. The perineal nerve may provide sensory and motor information from the perineum and the labia or scrotum. The dorsal nerve may send sensory information, including but not limited to touch, pleasure, pain, to the skin of penis or clitoris.

The motor function of the pudendal nerve may control the movement of one or more muscles. The motor function of the pudendal nerve may control the movement one or more of anal sphincter muscles and urethral sphincter muscles. The anal sphincter muscles may aid in holding in and release of feces. The urethral sphincter muscles may aid in holding in and release of urine. The pudendal nerve may provide sensory information about touch, pleasure, pain, and temperature of various anatomy, including but not limited to penis, vagina, perineum, anus, and anal canal. Injuries to the pudendal nerve may result in one or more of loss of sensation in the nerve's distribution, fecal and urinary incontinence, sexual dysfunction, or a combination thereof.

### Pre-Conditions

The individual experiencing incontinence and being prepared for treatment by electrical nerve stimulation may undergo various pre-condition steps before the start of the implantation procedure. The implantation procedure may be performed in a sterile operating room environment with laminar flow or a similar condition. The sterile operating room environment may have limited entry and movement of personnel. The equipment used for the procedure, including but not limited to the introducer, may be sterilized prior to the procedure. The introducer may comprise materials compatible with standard sterilization procedures, including but not limited to ethylene oxide gas, gamma irradiation, and autoclave sterilization. The surgical table may be capable of various patient positioning and X-ray C-arm access. In some embodiments, a radiographer, also known as radiologic technologist, may be present during the procedure to work with an image intensifier. One or more non-invasive imaging methods may be used along the anatomical path of the introducer during the implantation procedure to provide images of one or more of the anatomy, needle insertions, the introducer, the electrodes, and/or the leads. A patient controller may be fully charged and linked to the IPG prior to surgery. The IPG may be linked and charged through its packaging to maintain sterility. In some embodiments, the patient controller may be placed in a sterile bag and linked during surgery.

### Patient Preparation

The individual may be prepared for treatment by electrical nerve stimulation before the start of the implantation procedure. The IPG implantation site may be pre-marked in relation to posture and clothing to increase comfort for the individual after the procedure in their daily lives. The individual may be given a general anesthetic prior to the procedure. The individual may not receive any muscle relaxant drugs in preparation for the procedure. In some embodiments, the individual may be adequately positioned in a prone position, e.g., prone jack-knife, to allow surgical access. In some embodiments, left/right sided tilting and correct position may be checked prior to the procedure. A urethral transducer may be inserted in the individual to monitor and improve the accuracy of lead placement. In some embodiments, a transducer on urinary catheter may be used to monitor the progress of the lead placement. A transvaginal or transanal probe may be inserted safely into vagina of the individual for the purposes of measuring EMG. The transvaginal probe may be used to monitor and improve the accuracy of the lead placement. The transvaginal probe may additionally, or alternatively, be used to monitor the progress of the lead placement. An electrical ground pad may be placed on the individual away from surgical site. The skin of the individual, including but not limited to vaginal introitus, may be prepared, and draped prior to the procedure to reduce infection and surgical complications. A needle electrode may be inserted into the external anal sphincter of the individual for the purposes of measuring EMG. In some embodiments, an adhesive surface electrode may be applied onto the peri-anal skin of the individual for the purposes of measuring EMG.

In preparation for the implantation procedure, the individual may be positioned on an operating table in a prone position or a supine position. Since each patient may have a different constitution, the pelvis of each individual may have a different tilt when lying on an operating table. Thus, a method for standardizing the pelvic tilt of each individual patient while lying on an operating table may be beneficial for the correct positioning of the electrode leads during a lead implantation procedure. The clinician may take an x-ray image of the patient in a posterior-anterior (PA) view, or an anterior-posterior (AP) view, and use bone landmarks to analyze the x-ray image to determine whether a corrective action is necessary. The corrective action may comprise adjusting an operating table, e.g., left and right side tilt and/or head and foot end tilt, to alter an orientation of the pelvis. One or more corrective actions may be taken to achieve a target pelvic tilt. However, in some embodiments, no corrective action may be necessary. Although embodiments herein are discussed with respect to x-ray images, in some embodiments, another type of medical imaging may be used, e.g., CT scan, MRI scan, ultrasound imaging, etc.

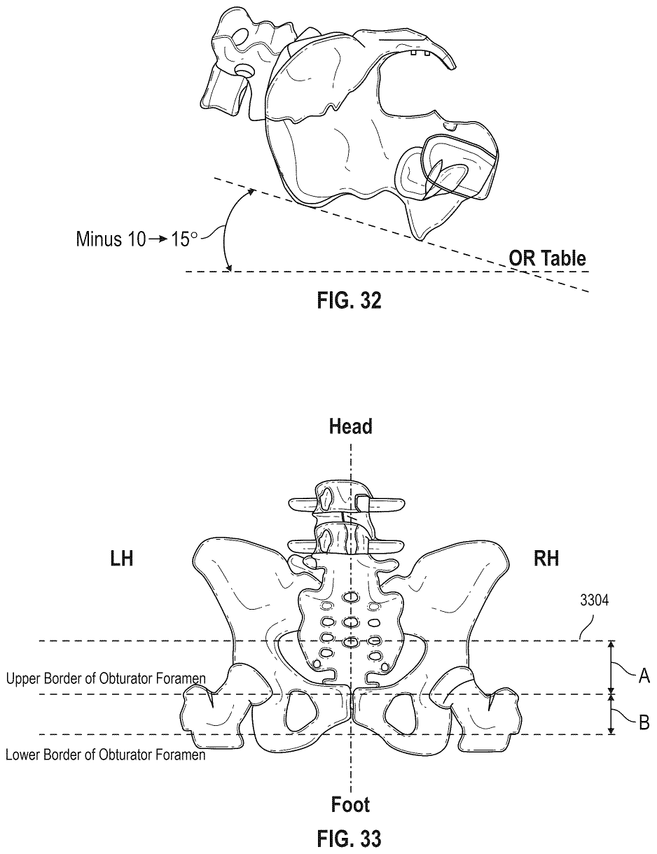

As illustrated in FIG. 32, a target pelvic tilt may comprise an angle of, for example, between −5 and −20 degrees, such as −5 degrees, −7 degrees, −10 degrees, −12 degrees, −15 degrees, −18 degrees, −20 degrees, or other values within the range. The target pelvic tilt may vary between different approaches for lead implantation. For example, the target pelvic tilt may be between −10 and −15 degrees for a ischiorectal approach. The target pelvic tilt may be different for a lower gluteal approach than for an ischiorectal approach for lead implantation.

In some cases, the manual process for standardizing a position of the patient on the operating table may be cumbersome for a clinician to perform during a medical procedure. Determining the pelvic tilt, or other asymmetry, of an individual patient may rely solely on the clinician's observations and may not be completely accurate at all times. The measurements relating to the patient anatomy, and the further instructions for adjusting the operating table, may require complex geometrical calculations that may be impractical for a clinician to perform in real time. The challenges of this process may lead clinicians to make errors, or to accept suboptimal positioning. In a procedure such as lead implantation, where precise positioning of the electrode lead at a target nerve is important for the desired results of neuromodulation, it may be increasingly important to have an accurate and/or automated patient preparation process. The automation may also decrease the overall time of the procedure, by eliminating the iterative steps that may be taken by a physician in trial-and-error positioning.

A control unit may be used to cause the corrective action based on one or more parameters from the x-ray image, or any other type of medical image disclosed herein. The control unit may include one or more actuators and a computing system. The one or more actuators may be mechanically coupled to at least the adjustment mechanisms of the operating table configured to control left and right side rotation of the table and/or tilting at the head and foot end of the table. In particular, the one or more actuators may lower one of the right or left sides of the operating table when a longitudinal symmetry of the x-ray image is poor. Additionally, or alternatively, the one or more actuators may lower one of the head or foot ends of the operating table when a pelvic tilt shown by the x-ray image is not within a target range.

The computing system may be configured to automatically determine key landmarks in the medical image. For example, the computing system may be configured to automatically determine the key bone landmarks in the x-ray image. The computing system may use artificial intelligence and/or machine learning to identify the landmarks. In some embodiments, the computing system may use image processing methods, e.g., edge detection, or other computer vision techniques, e.g., feature detection, for identifying the landmarks. The computing system may be trained on a database of x-ray images of the target anatomy labelled with the key landmarks. For example, the computing system may be trained on a set of x-ray images of the pelvis that have been labelled to identify the borders of the left and right obturator foramen and/or the lower margins of the sacroiliac joint. The computing system may then receive an x-ray image and provide it to the trained artificial intelligence model as an input. In response, the trained artificial intelligence model may output the x-ray image with markings identifying the key landmarks. The computing system may additionally, or alternatively, determine a ratio based on distances between the landmarks. The computing system may determine a corrective action based on a comparison of the determined ratio to a predetermined target ratio range. The computing system may additionally, or alternatively, determine a degree of longitudinal symmetry of the x-ray image based on a size and/or shape comparison of corresponding landmarks on the left and right side of the x-ray image. The computing system may determine a corrective action based on the determined degree of longitudinal symmetry. The computing system may cause an actuator to adjust the orientation of the operating table according to one or more determined corrective actions.

The x-ray images may demonstrate how the pelvis is tilted or inclined on the operating table. The clinician or a computing system identify one or more landmarks on the x-ray images used to assess the pelvic tilt of the individual. The landmarks may include specific bone structures. For example, as shown for the AP view model of the pelvis illustrated in FIG. 33, a first landmark may include a lower margin of the sacroiliac joint, and a second and third landmark may include the upper and lower borders of an obturator foramen. The one or more landmarks may each have a corresponding axis associated with the landmark. For example, each landmark may be marked with a horizontal axis crossing through the landmark on a left and right side of the image. As illustrated in FIG. 33, a first axis 3304 may be defined by a line crossing the lower margins of the sacroiliac joint on the left and right sides. A second axis 3308 may be defined by a line crossing the upper border of the left and right obturator foramina. A third axis 3312 may be defined by a line crossing the lower border of the left and right obturator foramina. In some embodiments, a computing system may be configured to automatically identify the one or more landmarks and/or corresponding axes in an x-ray image. The computing system may use a trained artificial intelligence model to identify the landmarks and/or corresponding axes. The computing system may be configured to output an x-ray image with markings denoting the landmarks and/or corresponding axes for the clinician to observe and analyze.

As illustrated by FIG. 33, a ratio may be determined by comparing a first distance A defined by the distance between the first and second axes 3304, 3308 to a second distance B defined by the distance between the second and third axes 3308, 3312. The determined ratio of the first distance to the second distance, also known as a tilt ratio, may indicate whether a corrective action at the head or foot end of the operating table is necessary. A target ratio range may be between 1 and 1.5, such as 1-1.05,1.05-1.10, 1.10-1.15, 1.15-1.20, 1.20-1.25, 1.25-1.30, 1.30-1.35, 1.35-1.40, 1.40-1.45, 1.45-1.50, or a range defined by any of these values. For example, a target ratio range may be between 1.0-1.25. If the tilt ratio is within the target ratio range, a corrective action at the head or foot end may not be necessary. If the tilt ratio is lower than the target ratio range, a corrective action may be necessary. For example, the corrective action may comprise lowering the head end of the operating table. Similarly, if the tilt ratio is higher than the target ratio range, a corrective action may be necessary. For example, the corrective action may comprise lowering the foot end of the operating table. In some embodiments, the computing system may be configured to automatically calculate the tilt ratio and determine whether a corrective action is necessary, and if so, determine which corrective action is appropriate. In some embodiments, the computing system may be configured to cause an actuator of the operating table to adjust the operating table based on the determined corrective action.

Additionally, or alternatively, an x-ray image may be used to identify whether a corrective action comprising adjusting the left or right tilt of the operating table is necessary. With an x-ray image of the relevant area of the patient body for lead implantation, a clinician or the computing system may identify a first landmark on a left side of the body and a second corresponding landmark on the right side of the body. For example, the first and second landmarks may include the left and right obturator foramen. The clinician or computing system may then identify a midline of the patient in the x-ray image and determine whether the first and second landmarks are adequately mirrored across the midline. Based on a determined degree of longitudinal symmetry, the clinician or the computing system may determine whether the operating table needs to be adjusted by rotating to the left or right side. If the landmarks are determined to be adequately symmetrical, no corrective action on the left or right tilt may be necessary. The longitudinal symmetry of the first and second landmarks may be assessed by observing on the x-ray image whether the landmarks are sufficiently equal in size and shape on each lateral side. For example, the obturator foramina provide easily observable and comparable landmarks on each lateral side of the body to determine whether the longitudinal symmetry of the patient is adequate for the implantation procedure. In some embodiments, the computing system may use a trained machine learning and/or artificial intelligence model to identify the landmarks on an x-ray image and provide a visual marker on or around the landmark, e.g., a borderline, for the clinician to visualize. The computing system may use the trained machine learning and/or artificial intelligence model to further analyze a size, shape, or other visual parameters of the landmarks to automatically determine whether the longitudinal symmetry of the patient is acceptable, and if corrective action is necessary. In some embodiments, the computer system may use image processing techniques or other computer vision techniques for determining a size, shape, or other visual parameters of the landmarks. For example, the computing system may calculate the areas of the left and right obturator foramen and determine whether a difference in the areas is higher than a predetermined threshold. If the difference in the areas of the foramen is higher than the threshold, a corrective action may be necessary. The computing system automatically performing the image analysis described herein may be beneficial as the analysis may be too complex and time-consuming for a surgeon to perform as part of preoperative procedures.

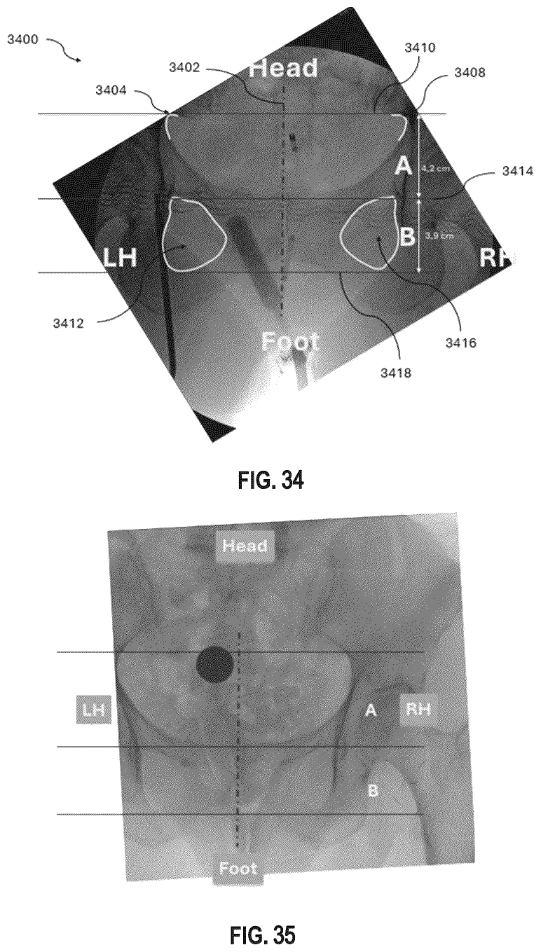

With respect to FIG. 34, an AP view x-ray image of the pelvis is shown with markings identifying bone landmarks, as described in some embodiments herein. The midline 3402 of the patient may be identified and may be marked on the image with a dotted line. The first landmark corresponding to the lower margin of the sacroiliac joints 3404, 3408 on each of the left and right side of the image may be identified. A first axis 3410 may be defined by the line crossing both points 3404, 3408 of the first landmark identified on each lateral side of the body. The clinician may also identify the right and left obturator foramina 3412, 3416, which are shown circled on the left and right hand sides of the image, respectively. A second landmark may comprise the upper border of the obturator foramina, and a third landmark may comprise the lower border of the obturator foramina. The second and third axes 3414, 3418 may be defined by the lines crossing both points of the upper and lower borders of the foramina, respectively, on the left and right hand sides of the image. The first, second, and third axes 3410, 3414, 3418 may be substantially parallel to each other. The size and/or shape of the left and right obturator foramina may be compared to determine whether they are sufficiently similar to indicate an acceptable longitudinal symmetry. In the illustrated example of FIG. 34, the left obturator foramen 3416 and the right obturator foramen 3412 are sufficiently similar in shape and size to indicate a good longitudinal symmetry. Thus, no corrective action to the left or right rotation of the operating table would be necessary. A pelvic tilt ratio may then be determined from the x-ray image, the tilt ratio comprising the ratio of the distance A to the distance B, wherein A is the distance between the first and second axes 3410 and 3414, and B is the distance between the second and third axes 3414 and 3418. As illustrated in FIG. 34, the distance A is measured as 4.2 cm, and the distance B is measured as 3.9 cm, resulting in a pelvic tilt ratio of about 1.08. A target pelvic tilt ratio range may be about 1 to 1.25, which encompasses the determined tilt ratio of 1.08. Thus, no corrective action to the head and foot ends of the operating table would be necessary. The pelvic tilt represented by the image in FIG. 34 may be −12.5 degrees, which may be within a target range of pelvic tilt, such as −10 degrees to −15 degrees.

FIG. 35 illustrates an example AP view x-ray image showing when no corrective action is required. The left and right obturator foramina may be identified. A clinician or a computing system may determine that a longitudinal symmetry of the left and right foramina is acceptable, as described elsewhere herein. Thus, no corrective action to adjust the left or right rotation of the operating table may be necessary. The first, second, and third axes associated with the first, second, and third bone landmarks, respectively, may be identified and marked, as discussed elsewhere herein. The distances between the axes may be used to determine a tilt ratio, as discussed elsewhere herein. With respect to FIG. 35, the tilt ratio may be determined to be 1.25. The tilt ratio of 1.25 may be determined to be within the target range, e.g., 1 to 1.25. Since the tilt ratio is within the target range, no corrective action may be required to adjust the head or foot ends of the operating table. The pelvic tilt for the x-ray image shown in FIG. 35 may be −10 degrees, which may be within a target range of pelvic tilt, as described elsewhere herein.

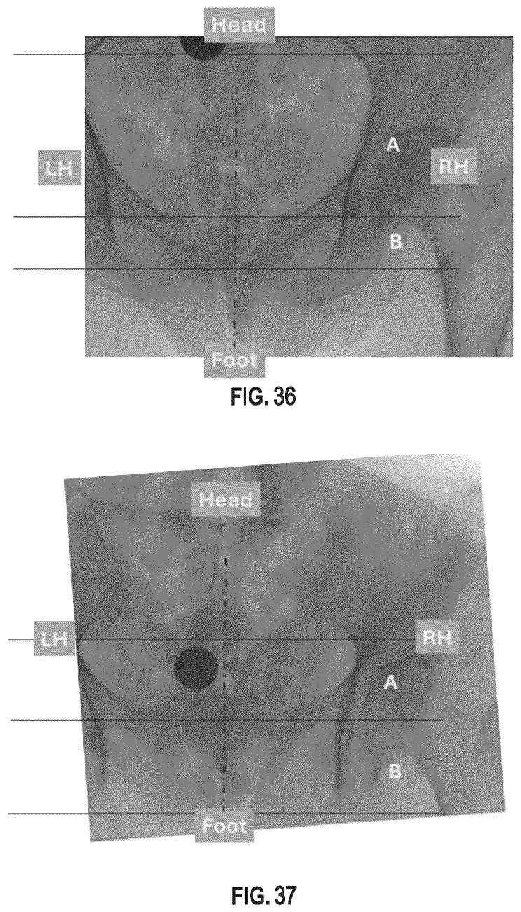

FIGS. 36 and 37 illustrate AP view example x-ray images showing when corrective action is necessary for adjusting a head or foot end of the operating table to adjust the pelvic tilt. In both examples, the longitudinal symmetry was determined to be acceptable based on the similar size and shape of the obturator foramina. The tilt ratio may be calculated based on first, second, and third axes of corresponding identified bone landmarks, as described elsewhere herein. When the determined tilt ratio exceeds a target range, a corrective action may comprise lowering the foot end of the operating table. For example, with respect to FIG. 36, the tilt ratio may be determined, as described elsewhere herein, as 3, which may be higher than the predetermined target range, e.g., 1 to 1.25. Thus, the corresponding corrective action determined from the x-ray image of FIG. 36 may comprise lowering the foot end of the table. When the tilt ratio falls below the target range, a corrective action may comprise lowering the head end of the operating table. The pelvic tilt for the x-ray image shown in FIG. 36 may be +10 degrees, which may be outside a target range of pelvic tilt, e.g., −10 to −15 degrees, as described elsewhere herein, thus requiring a corrective action. For example, with respect to FIG. 37, the tilt ratio may be determined, as described elsewhere herein, as 0.85, which may be lower than the predetermined target range, e.g., 1 to 1.25. Thus, the corresponding corrective action may comprise lowering the head end of the table. The pelvic tilt for the x-ray image shown in FIG. 37 may be −20 degrees, which may be outside a target range of pelvic tilt, e.g., −10 to −15 degrees, as described elsewhere herein, thus requiring a corrective action.

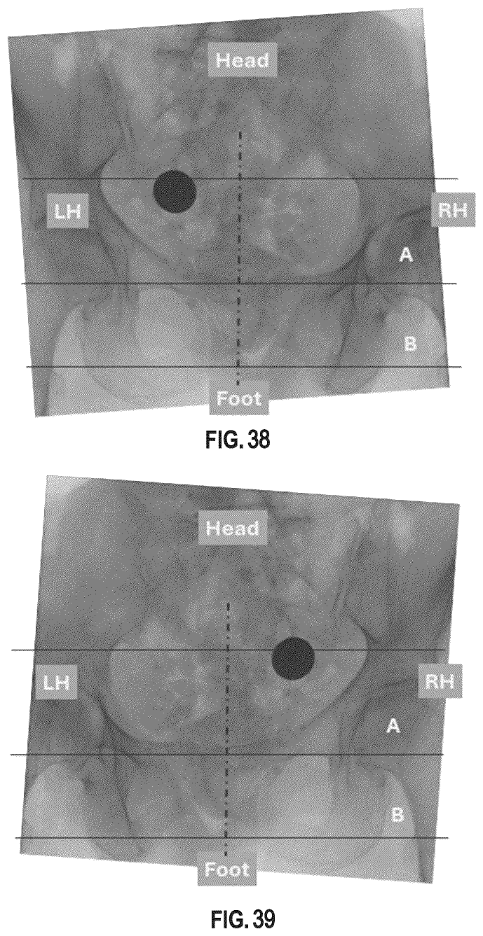

FIGS. 38 and 39 illustrate example AP view x-ray images showing when corrective action is necessary for adjusting a left or right rotation of the operating table to achieve longitudinal symmetry. The clinician or a computing system may identify a first landmark on a left side of the image and a corresponding second landmark on a right side of the image, as described elsewhere herein. For example, for an x-ray image of the pelvis, the first and second landmark may comprise the left and right obturator foramina. The first and second landmarks may be compared for shape, size, position, etc. to determine whether a degree of longitudinal symmetry of the x-ray image is adequate. In some embodiments, a size or shape comparison may be automatically performed using a machine learning and/or artificial intelligence model. For example, with respect to FIG. 38, the longitudinal symmetry is poor because the obturator foramen on the right hand side is noticeably smaller than the obturator foramen on the left hand side. In such a case, a corrective action may comprise lowering the right hand side of the operating table, e.g., by a rotation of 10 degrees. In another example, with respect to FIG. 39, the longitudinal symmetry is poor because the obturator foramen on the left hand side is noticeably smaller than the obturator foramen on the right hand side. In this case, a corrective action may comprise lowering the left hand side of the table, e.g., by a rotation of 10 degrees.

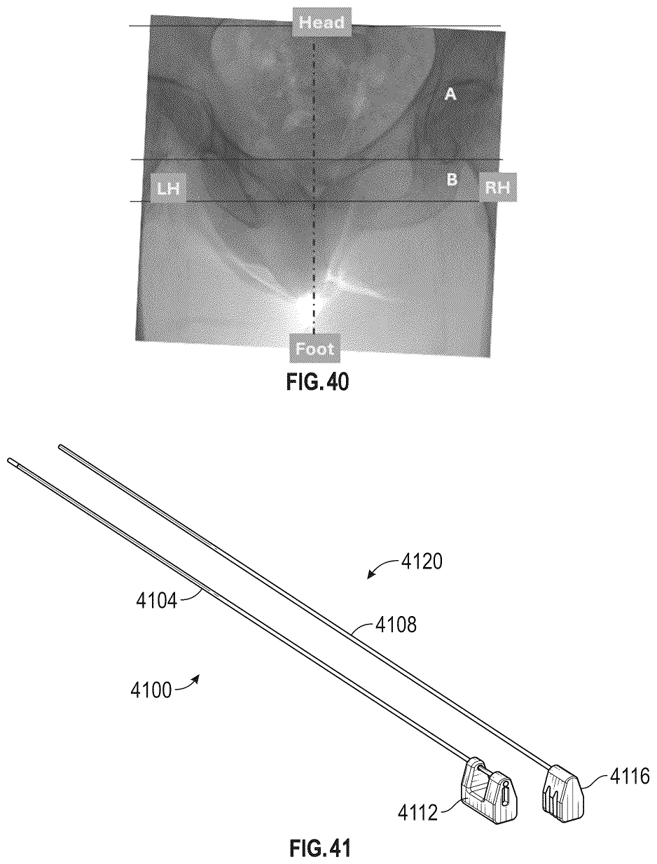

FIG. 40 illustrates an example AP view x-ray image showing when more than one corrective action is necessary. The clinician or a computing system may identify a first landmark on a left side of the image and a corresponding second landmark on a right side of the image to determine a degree of longitudinal symmetry, as described elsewhere herein. With respect to FIG. 40, the longitudinal symmetry may be poor because the obturator foramen on the left hand side is noticeably smaller than the obturator foramen on the right hand side. Thus, a first corrective action may comprise lowering a left hand side of the operating table, e.g., by a rotation of 10 degrees. The tilt ratio may also be calculated based on first, second, and third axes of corresponding identified bone landmarks, as described elsewhere herein. With respect to FIG. 40, the tilt ratio may be determined as 3.15, which may be higher than the predetermined target range, e.g., 1 to 1.25. The pelvic tilt for the x-ray image shown in FIG. 37 may be +10 degrees, which may be outside a target range of pelvic tilt, e.g., −10 to −15 degrees, as described elsewhere herein, thus requiring a corrective action. Thus, a second corrective action may comprise lowering a foot end of the operating table. The first and second corrective actions may be performed in any order, or simultaneously.

### Nerve Localization and Lead Placements

Provided herein are methods, devices, and systems to allow for accessing the pudendal nerve by one or more anatomical paths with little damage to tissues surrounding the pudendal nerve. Often, accessing the pudendal nerve and placing one or more leads with minimal injury to the surrounding tissues may be difficult by the anatomical structure near the pudendal nerve and because the pudendal nerve may run in three different planes of the body. For example, the pudendal nerve runs in a broadly caudad course on the inner side of the ischial bone and then turns forward to the ischiorectal fossa. Described herein are imaging-guided markings on skin to guide the anatomical path of the introducer to access the pudendal nerve. Radiological images of the gluteal area of the individual may be taken with a metal guide placed on the skin. The radiological images may be used to determine the locations of a series of surface markings on the skin in the gluteal region to provide the direction of the anatomical path of the introducer in the individual to access the pudendal nerve. The radiological images may be taken by fluoroscopy. In some embodiments, the radiological images may be additionally, or alternatively taken by x-ray. One or more electrode leads may be placed at or near a pudendal nerve trunk. Additionally, or alternatively, one or more electrode leads may be placed at or near the anterior branches of the pudendal nerve. The introducer may access the pudendal nerve by an ischiorectal approach, where the introducer is directed to penetrate or pass close to the sacrotuberous ligament and place the lead near the pudendal nerve trunk at a target location proximal to Alcock's canal in the region of the ischial spine. In some embodiments, the introducer may access the pudendal nerve by a low gluteal approach, where the introducer is directed to pass in the space between sacrotuberous ligament and sacrospinous ligament and to pass anteriorly in the ischiorectal fossa below the pelvic floor to place the lead on the anterior branches of the pudendal nerve to stimulate the pudendal nerve and the dorsal genital nerve.

Localizing a target nerve prior to insertion of any leads may beneficially allow for the one or more leads to be placed in an optimal position for stimulation and/or sensing of muscle activity. For example, localizing the pudendal nerve may allow for leads to be positioned more accurately and parallel to the pudendal nerve (e.g., at the pudendal nerve trunk). The placement of the lead parallel to the pudendal nerve trunk, rather than intersecting the nerve at a single point, may allow for a greater length of interaction between the electrode lead and the nerve such that each electrode along a length of the lead may be in an optimal range and/or position for stimulating the nerve. The lead may also be in a beneficial position close to the pelvic floor and/or obturator fossa to record pelvic floor muscle activity and/or obturator muscle activity. The nerve may be localized using one or more needles. The one or more needles may include a marking needle and/or a stimulating member (e.g., stimulating needle). The marking needle may indicate a horizontal and/or a vertical level of a nerve targeted for stimulation, such as the pudendal nerve. The marking needle may be inserted using one or more lines, such as a first line and a second line. The marking needle may be inserted at or near the intersection of the first line and the second line. The first line and second line may comprise one or more surface markings drawn on the skin. One or more of the surface markings may be guided by imaging. For example, the patient or subject may be placed in a prone position and the surface markings may be drawn using radiology techniques such as x-ray or fluoroscopy. In some embodiments, the surface markings may be guided by palpating the gluteal region. In some embodiments, the surface markings may comprise a radiopaque marker. In some embodiments, the approach may utilize two stimulating members. A first stimulating member may be used to mark the nerve and will generally intersect the nerve. A second stimulating member may be used to approach generally parallel to the pudendal nerve and place the lead parallel to the pudendal nerve as described above.

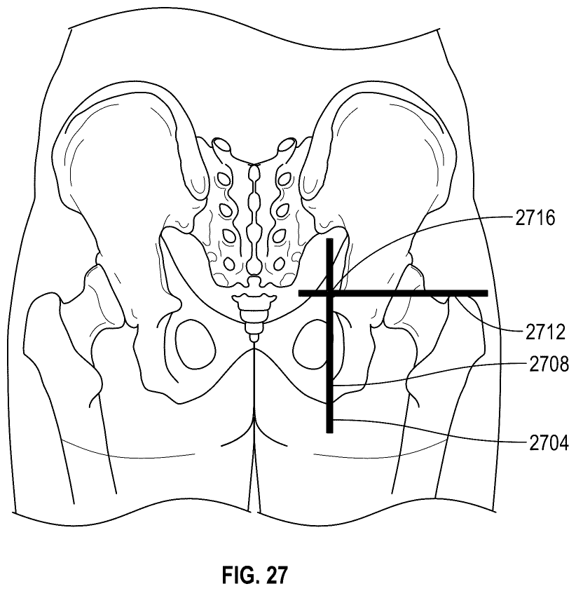

FIG. 27 shows a schematic of surface markings drawn on an individual to locate the pudendal nerve. A surface marking 2704 may be made by palpating the ischial tuberosity and marking the ischial tuberosity. An anterior-posterior x-ray image or fluoroscopy may be used to draw a vertical line surface marking 2708 corresponding to the inner rim of the ischial bone. A similar technique may be used to draw a horizontal line surface marking 2712 through the top of the greater trochanter and intersecting the vertical line surface marking 2708. The intersection 2716 between the two surface markings may indicate a position closely related to the ischial spine. In some embodiments, the one or more surface markings may be drawn on the contralateral side in a similar manner. The marking needle may be inserted perpendicularly into a position indicated by an intersection of one or more surface markings. In some embodiments, the marking needle may be inserted into a position adjacent an intersection of the surface markings. For example, the marking needle may be inserted at a position about 1 cm lateral to the intersection 2716.

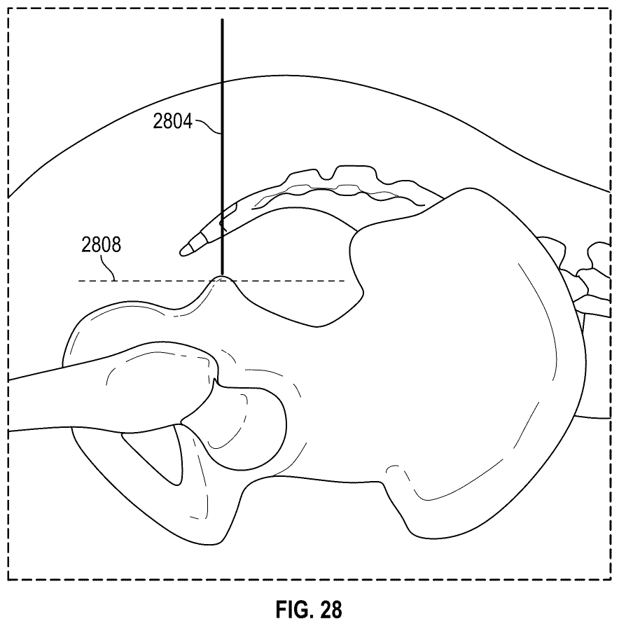

FIG. 28 shows a schematic of a lateral view of the anatomy and an inserted marking needle in an individual. The marking needle 2804 may be inserted until it comes into contact with the ischial bone to indicate a horizontal level of the ischial spine for guidance on a lateral x-ray. Thus, the tip of the marking needle 2804 may be easily visualized in a lateral view to indicate a vertical level 2808 at which the pudendal nerve turns inward at the ischial spine. The marking needle 2804 may have a length greater than the distance from an insertion point to the ischial spine of a specific individual. In some embodiments, the length of the marking needle 2804 may comprise about 6 cm to 20 cm. In some embodiments, the length of the marking needle 2804 may comprise about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, or a length in a range defined by any of these values. If the marking needle is a stimulating needle, the tip of the stimulating needle will mark the vertical level where the pudendal nerve runs when activate external anal sphincter EMG activity. The stimulating needle may be advanced slowly, just medial from the transecting point of the x-ray lines, and perpendicular to the skin. With stimulation (e.g., less than or equal to 3 mA or less than or equal to 2 mA), there may be an EMG response of the external anal sphincter and/or pelvic floor when the needle tip marks the vertical level (in lateral X-ray view) of the pudendal nerve. In other approaches, the marking needle 2804 may be introduced at an oblique angle with the needle at the inner side of the internal obturator muscle and next to Alcock's canal.

The one or more needles used to localize a target nerve may comprise a stimulating needle, or a stimulating needle assembly as described elsewhere herein. The stimulating needle, or stimulating needle assembly, may also be referred to elsewhere herein as a stimulation needle, or stimulation needle assembly. The stimulating needle may define the pathway for the introducer and/or leads for implantation at the target nerve. For example, the target nerve(s) may comprise one or more of the pudendal nerve, tibial nerve, peroneal nerve, sacral nerve, cauda equina, pelvic parasympathetic nerves, lumbar sympathetic nerves, and/or nerve branches leading to and from such nerves. The stimulating needle may be used to deliver a constant low level of stimulation while inserted into an individual, such that a bioelectrical response, e.g., EMG, EEG, ECG, etc., of the individual may be measured. For example, the stimulating needle may be used for the purpose of measuring EMG response to monitor and verify a desired pathway for the lead to be implanted. To reach the pudendal nerve, the stimulating needle may be inserted using an ischiorectal approach, starting in the ischiorectal fossa and medial to the ischial tuberosity. The stimulating needle may be advanced generally in a cephalad direction, passing through the lesser sciatic foramen toward the ischial spine. The stimulating needle may penetrate the skin about 5 mm to 10 mm medial to the ischial tuberosity, at a level determined by the marking needle. Under an ischiorectal approach, the stimulation needle may be directed and advanced in a horizontal plane toward the tip of the marking needle using a lateral view x-ray as guidance. Once the initial orientation of the stimulating needle in a lateral view x-ray is assured to be about level with the tip of the marking needle, the stimulating needle may be further directed and advanced using the guidance of an anterior-posterior view x-ray toward the tip of the marking needle.

The stimulating needle may be connected to an external stimulator. The stimulator may provide about 6 mA or less of current to the stimulating needle. The stimulator may provide a current of about 1 mA, 2 mA, 3 mA, 4 mA, 5 mA, or 6 mA. Using higher stimulation currents may result in nonselective contractions throughout the surrounding region, inhibiting the effectiveness of measuring EMG response as a positioning tool. The threshold for a measured EMG response indicating proper placement may be in a range between 10 μV and 30 μV. For example, the threshold EMG response may be about 10 μV, 12 μV, 14 μV, 16 μV, 18 μV, 20 μV, 22 μV, 24 μV, 26 μV, 28 μV, or 30 μV. The threshold EMG response may be 20 μV or greater. Pudendal nerve stimulation advantageously may provide a response in the external anal sphincter (EAS), external urethral sphincter (UES), and/or the pelvic floor, while sacral nerve stimulation may only result in a pelvic floor response. For locating the pudendal nerve, the stimulating needle may be advanced in a cephalad trajectory using an ischiorectal approach, and an EMG response may be measured in at least the EAS and pelvic floor to verify correct placement at the pudendal nerve. The EMG response in the EAS and/or UES may be measured using one or more recording, or sensing, needles. The EMG response in the pelvic floor may be measured using a transvaginal probe or an EMG needle (e.g., passing lateral of the anus until pelvic floor activity is measured). As the stimulating needle is advanced, the pathway of the needle stimulation may comprise directly stimulating the pelvic floor, then fatty tissue, and then stimulating the pudendal nerve, in that order. Thus, the order of the corresponding detected EMG response may comprise pelvic floor only, then no response, followed by EAS and/or pelvic floor response. An optimal placement of the stimulating needle at or near the pudendal nerve may result in both an EAS and a pelvic floor EMG response, indicating effective stimulation of the pudendal nerve. In some embodiments, an optimal placement of the stimulating needle may result in an EAS response but no pelvic floor response. If there is no measured EAS response, the stimulating needle placement may be adjusted axially and/or vertically, guided by imaging. A placement of the stimulating needle resulting in only a pelvic floor response may be acceptable if the needle position has been adjusted multiple times without resulting in an EAS response. For example, if the stimulating needle position is adjusted at least 5 times without achieving an EAS response, then a stimulating needle position giving only a pelvic floor response may be acceptable. Once the proper EMG response is obtained, the position of the stimulating needle may be confirmed by a lateral x-ray and/or the marking needle. A well-positioned stimulating needle for the pudendal nerve may be just posterior to the ischial spine and just medial to the marking needle. In some methodologies, a position of the stimulating member may be confirmed based only on an EMG response at the external anal sphincter.

The stimulating and/or marking needle may be used with a removable stylet. Some needles may comprise an open bore without a removable stylet. When a needle with a lumen and without a stylet is inserted into a patient, there may be blood or tissue that clogs the lumen of the needle and prevents proper insertion of the needle. Thus, a needle with a removable stylet may allow for insertion of the needle without substantial interference from blood or tissue. The stylet may also provide additional rigidity to the needle during insertion, such that the needle body does not deflect or bend upon insertion. Once inserted, the stylet may optionally be removed to aspirate or withdraw any fluid or tissue.

FIG. 41 illustrates a stimulating needle assembly comprising a stimulating needle 4100 and a removable stylet 4120. The stimulating needle 4100 may comprise an elongated needle body 4104 and a needle grip 4112. The removable stylet 4120 may comprise an elongated stylet body 4108 and a stylet grip 4116. The grip of the needle or stylet may also be referred to herein as a hub. The needle body 4104 may comprise an inner lumen. The needle body 4104 may comprise a proximal opening leading to the inner lumen. The needle body 4104 may or may not comprise a distal opening. The stimulating needle 4100 may comprise a distal tip. The distal tip may allow the needle to be advanced through tissue, unlike a guidewire. In some cases, the needle 4100 may not comprise a distal tip. The stylet body 4108 may be configured to be inserted into the inner lumen of the needle body 4104. The stylet 4120 may or may not comprise a distal tip. The stylet body 4108 may be inserted into the proximal opening of the needle body 4104 at a proximal end of the needle grip 4112 and into the inner lumen of the elongated needle body 4104.

The stimulating needles disclosed herein may be configured to conduct stimulation from a proximal connection portion to a distal stimulating portion to deliver stimulation to an individual only from a specific region of the needle, e.g., at or near the distal end. The connection portion may always be proximal to the stimulating portion. For example, referring to FIG. 41, the needle body 4104 may be comprised of a conductive material. The needle body 4104 may comprise one or more insulated portions, e.g., conductive material coated in an insulating material, along a length of the needle body 4104. The insulated portions may comprise insulating coating, e.g., polymer coating, on the outer needle body and the inner lumen. The needle body 4104 may comprise an uninsulated stimulating portion for delivering stimulation at or near a distal end of the needle 4100, e.g., at a distal tip or at a distal electrode. In some embodiments, the needle 4100 may include one or more distal electrodes. In some embodiments, the needle 4100 may include one or more electrodes that partially extend around the outer circumference of the needle body, e.g., to deliver targeted stimulation. The needle body 4104 may also comprise an uninsulated connection portion for connection to an external source of stimulation. The connection portion may be located at or near a proximal end of the needle body 4104. As illustrated in FIG. 41, the connection portion of the stimulating needle 4100 is located at the proximal end of the needle body 4104 with the needle hub 4112. However, in some embodiments, the connection portion may be located in a middle portion of the needle body, as disclosed elsewhere herein. Any of the stimulating and/or marking needles disclosed herein may include one or more depth markings along a length of the needle body.

The stimulating needle 4100 and/or marking needle may comprise a connection region, or connection portion, configured to receive or interface with an electrical connector. The connection region may be an outer portion of the elongate needle body of the needle. For example, a J-clip may be used to interface the connection region of the needle with a source of electrical stimulation. The source of electrical stimulation may comprise an external stimulator providing a stimulation current. The J-clip may be configured to hook around the outer portion of the needle body. Some stimulating needles may not have a well-defined and secure region for electrical connection. For these needles, a connection region must be identified by a user and an attachment to a J-clip or other connector device may not be completely secure, e.g., may slide off or slide onto an insulated portion. Thus, a needle with a well-defined and secure electrical connection region may be beneficial.

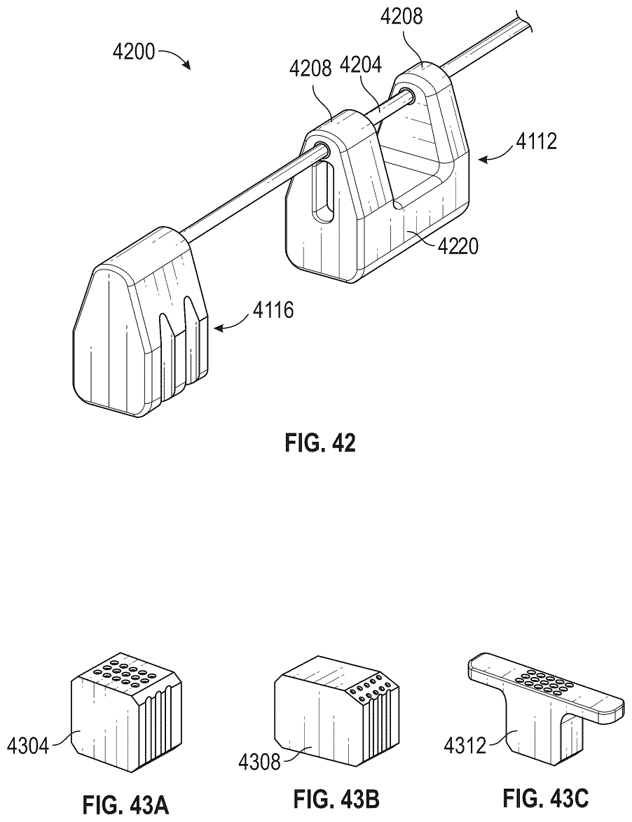

FIG. 42 illustrates a stimulating needle assembly 4200 comprising the needle 4100 (FIG. 41) with a needle grip 4112 and the stylet 4120 (FIG. 41) with a stylet grip 4116. The needle grip 4112 may comprise a grip body 4212 and two arms 4208 extending in a same direction from the grip body 4212. The two arms 4208 may extend from proximal and distal ends of the grip body 4212. The two arms 4208 of the needle grip 4112 may hold the elongate needle body 4104 and define a connection region 4204 on the needle body. The connection region 4204 may be configured to conduct current received from a connector coupled to an external stimulator, through the needle body 4104, to a distal stimulating region of the needle. For example, a J-clip may hook around the needle body to electrically connect an external stimulator to the stimulating needle 4100 at the connection region 4204. The space, or window, 4216 between the two arms 4208 and the grip body 4212 of the needle grip 4112 may be used for positioning the electrical connector, e.g., the J-clip, throughout the duration of the connection to the external stimulator. The arms 4208 of the needle grip 4112 may prevent the J-clip or other electrical connector from inadvertently sliding off of the needle. Furthermore, a user may be able to easily visualize the connection region 4204 and know where to connect the J-clip or other connector on the needle. The needle grip 4112 may be permanently attached to the needle body 4104. In some cases, the needle grip 4112 may be removable from the needle body 4104, as discussed elsewhere herein.

The stimulating and/or marking needle, including any of the embodiments of needles or needle assemblies disclosed herein, may be used with a needle guide. For example, a kit may be provided including a needle guide and a plurality of needles or needle assemblies, embodiments of which are described herein. When inserting the stimulation needle, a clinician may require multiple insertion attempts to find an optimal response, as discussed elsewhere herein. However, inserting and removing the needle multiple times in the same vicinity may be burdensome. For example, a clinician may determine that the needle needs to be only a small distance away, e.g., 1-3 mm, from the previous location. Thus, a needle guide may be configured to allow for easy insertion of a plurality of needles, or needle assemblies, in close proximity around a small area, instead of removing one needle and inserting it again. Furthermore, the needle guide may allow for a side-by-side comparison of a plurality of stimulation needles to determine which needle elicits the best response, as described elsewhere herein.

FIGS. 43A-43C illustrate various types of needle guides, such as a trunk needle guide 4304 (FIG. 43A), an anterior needle guide 4308 (FIG. 43B), and a T-handle needle guide 4312 (FIG. 43C). The needle guide may comprise a guide body comprising a plurality of channels formed within the body. Each of the plurality of channels may comprise a proximal opening on a proximal side of the body facing away from the patient and a corresponding distal opening on an opposing distal side of the body facing the patient. Each of the plurality of needles, or needle assemblies, may be inserted through a corresponding channel of the needle guide and into a patient. The channels of the needle guide may each be equidistant from adjacent channels to provide multiple options for finding the best spot to stimulate. For example, each opening of the channels may be spaced from each other by a distance of at least about 1 to no more than 5 mm, such as no more than 1 mm, no more than 2 mm, no more than 3 mm, no more than 4 mm, no more than 5 mm, or other values within the range. The plurality of channels may be formed substantially parallel to each other, so that the one or more needles may be kept parallel to each other upon insertion and do not inadvertently bend into each other. As shown in FIG. 43B, the channels of the anterior channel guide 4308 may be formed at an angle relative to the body of the needle guide, to allow for a standardized angular insertion of multiple needles. As shown in FIG. 43C, the T-handle needle guide 4312 may comprise a T-handle configured to be gripped by a user to allow for an easier grip when inserting the one or more needles into the needle guide.

The stimulating and/or marking needle and the corresponding stylets may include a low profile needle grip. Some stimulating needles include a large grip portion having a square or rectangular cross-section that makes it difficult to insert multiple needles in close proximity, e.g., within 1-3 mm of each other. The low profile needle grip may allow for insertion of multiple needles in close proximity with a needle guide. For example, a needle may comprise a tapered grip that does not interfere with the needle grips of an adjacent needle when used in a needle guide.

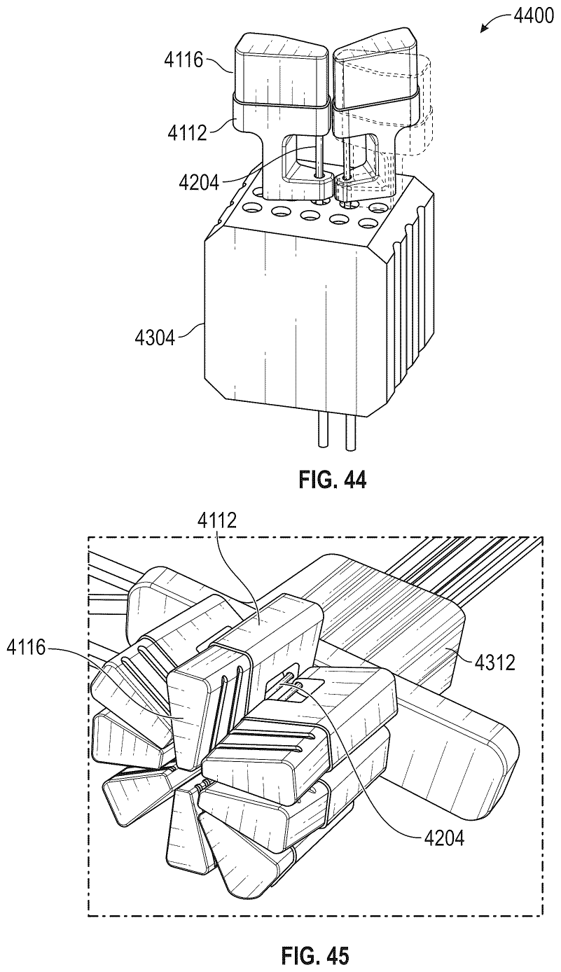

FIG. 44 illustrates an example of a needle guide assembly 4400 comprising a plurality of stimulating needle assemblies, e.g., stimulating needle assembly 4200 (FIG. 42), inserted into a needle guide 4304. The stimulating needle assembly may comprise a stimulating needle with a needle grip 4112, and a removable stylet with a stylet grip 4116. The needle grip 4112 may comprise a grip body that tapers toward a first or tapered end of the grip holding the needle body. For example, as shown in FIG. 42, the two arms 4208 of the needle grip 4112 may taper from the grip body 4220 towards an end of the grip where the needle body 4104 is attached. The stylet grip 4116 may have a cross-sectional shape corresponding to a cross-sectional shape of the needle grip 4112. The stylet grip 4116 and/or the needle grip 4112 may include gripping features formed on the grip, such as ridges or grooves. The needle grips of the plurality of needles inserted into the needle guide may be arranged circumferentially around the needle bodies of the plurality of needles. For example, as shown in FIGS. 44 and 45, each of the plurality of needles inserted into the needle guide may be positioned in the needle guide with the tapered end of the needle grip 4112 facing inward towards the tapered end(s) of the other needle grips. A needle guide and the tapered needle grips may allow for the insertion of 2-16 needles, such as 2 needles, 4 needles, 6 needles, 8 needles, 10 needles, 12 needles, 14 needles, 16 needles, or a value within this range, simultaneously within a needle guide. Advantageously, the connection regions 4204 of the each of the plurality of needles may each still be accessed from a window 4216 (FIG. 42) defined between the arms 4208 of the needle grip 4112, even when the needles are simultaneously inserted into the needle guide, allowing for side-by-side comparisons of the responses elicited by each needle to determine the optimal placement, as described elsewhere herein. As shown in FIG. 45, the needle grips 4112 of the eight needles inserted into the T-handle needle guide 4312 are arranged circumferentially around the needle bodies with the tapered end(s) of the needle grips 4112 facing inwards and the outer connection regions 4204 are accessible.

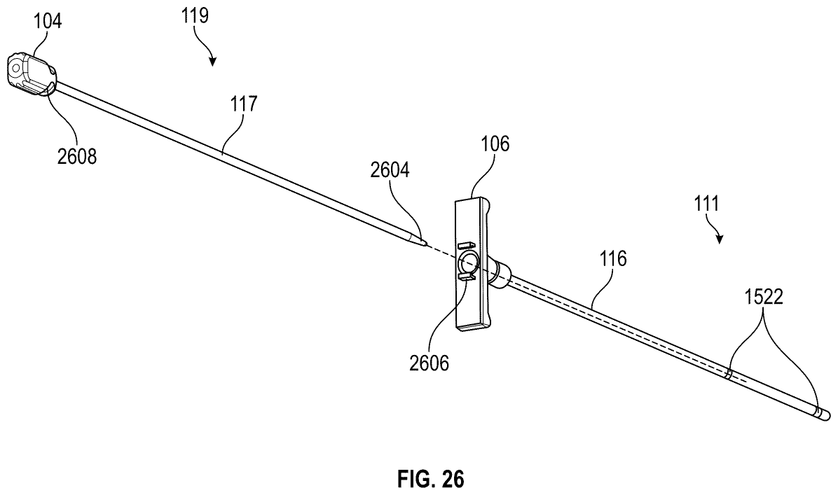

Once the stimulating needle is adequately positioned, a guidewire may be inserted fully through the stimulating needle. The guidewire may include markings that may prevent ingress of the guidewire into the deeper tissue. The markings may comprise graduated depth markings, wherein each marking is spaced by a same distance, e.g., 5 cm. The markings may comprise laser-made markings, dual color PTFE coatings. or textural differences on the guidewire. For example, the guidewire may be textured such that it alternates between a rough and smooth finish of the metal to create the markings. Haptic feedback or fluoroscopy may indicate when the guidewire has reached the distal end of the stimulating needle. The stimulating needle may then be retracted and removed from the patient. The guidewire should be carefully held in position, avoiding any further retraction or advancement. An introducer, such as the introducer assembly shown in FIG. 26, may then be placed over the guidewire and the guidewire may be removed, such that the lead may be inserted. The introducer assembly may comprise an introducer sheath and an obturator, such as the introducer 111 and obturator 119 shown in FIG. 26, as discussed elsewhere herein. The proper placement of the introducer may be confirmed by visualization of a radiopaque introducer sheath and/or one or more radiopaque markers on the introducer sheath. The lead may be implanted using the introducer and/or a lead positioning guide, using devices and methods described herein, such that the tip of the lead is implanted at the same position reached by the tip of the stimulating needle, e.g., parallel to the pudendal nerve.

In some embodiments, a guidewire may not be necessary for the placement of the electrode lead. A guidewire may move within the tissue during or after the removal of the stimulating needle from the tissue. A guidewire may not be able to be directly inserted into the tissue without a premade insertion path as it may lack the distal tip included on a needle. Furthermore, a surgeon may have to hold the guidewire steady in place as the introducer assembly is advanced over the guidewire. This may cause inadvertent migration and/or bending of the guidewire from the position determined by the stimulating needle. The guidewire may inadvertently lose the position determined by the stimulating needle. The stimulating needle, or stimulating needle assembly, may be more rigid than a guidewire. Furthermore, a stimulating needle may comprise a distal tip configured to pierce tissue to create a path into the pelvic area, which may include tough ligaments. The distal tip may comprise a beveled and/or faceted tip. The distal tip may be angled to facilitate introduction into the tissue. Thus, it may be beneficial to eliminate the step involving the guidewire by allowing the introducer assembly to be advanced directly over a needle or needle assembly into an individual. The increased stiffness of the stimulating needle assembly compared to a guidewire may also increase the stiffness of the introducer assembly as it is advanced over the stimulating needle assembly, e.g., through and/or past a ligament complex. In some embodiments, the stimulating needle assembly may also continue to provide stimulation to verify the position as the introducer is advanced into and through the tissue, while a guidewire may not be able to deliver said stimulation. In some embodiments, the stimulating needle may not have an inner lumen, as a guidewire may not be necessary in the implantation procedure. In some cases, the stimulating needle may have an inner lumen that is sized to not allow for the insertion of a guidewire or a stylet. The stimulating needle for use without a guidewire may have an increased rigidity as compared to a stimulating needle for use with a guidewire, as the inner lumen may be eliminated or significantly decreased in diameter. In some cases, the needle may not bend and may not be flexible. The needle may have an outer diameter of about 30 gauge to about 12 gauge. The needle may have an outer diameter of at least 28 gauge, at least 26 gauge, at least 24 gauge, at least 22 gauge, at least 20 gauge, at least 18 gauge, at least 16 gauge, at least 14 gauge, at least 12 gauge, or a diameter in a range defined by any of these values. The needle may have an outer diameter of less than 12 gauge, less than 14 gauge, less than 16 gauge, less than 18 gauge, less than 20 gauge, less than 22 gauge, less than 24 gauge, less than 26 gauge, less than 28 gauge, or a diameter in a range defined by any of these values. In some embodiments, where the needle has an inner lumen, a needle wall may have a thickness of at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, and/or less than 1.0 mm, less than 0.9 mm, less than 0.8 mm, less than 0.7 mm, less than 0.6 mm, less than 0.5 mm, less than 0.4 mm, less than 0.3 mm, less than 0.2 mm or a thickness in a range defined by any of these values. In some embodiments, where the needle has an inner lumen, the inner lumen diameter may be less than 0.1 mm, less than 0.2 mm, less than 0.3 mm, less than 0.4 mm, less than 0.5 mm, less than 0.6 mm, less than 0.7 mm, less than 0.8 mm, or a diameter in a range defined by any of these values.

The stimulating needles disclosed herein may be beneficial for locating a target nerve, e.g., the pudendal nerve or the sacral nerve, tibial nerve, peroneal nerve, cauda equina, pelvic parasympathetic nerves, lumbar sympathetic nerves, and/or nerve branches leading to and from such nerves in the pelvic area. For example, the pudendal nerve may be surrounded by tough tissue, e.g., sacrotuberous ligaments, for which it may be beneficial for the stimulating needle to have an increased rigidity to prevent and/or reduce deflection of the needle during insertion, as disclosed elsewhere herein.

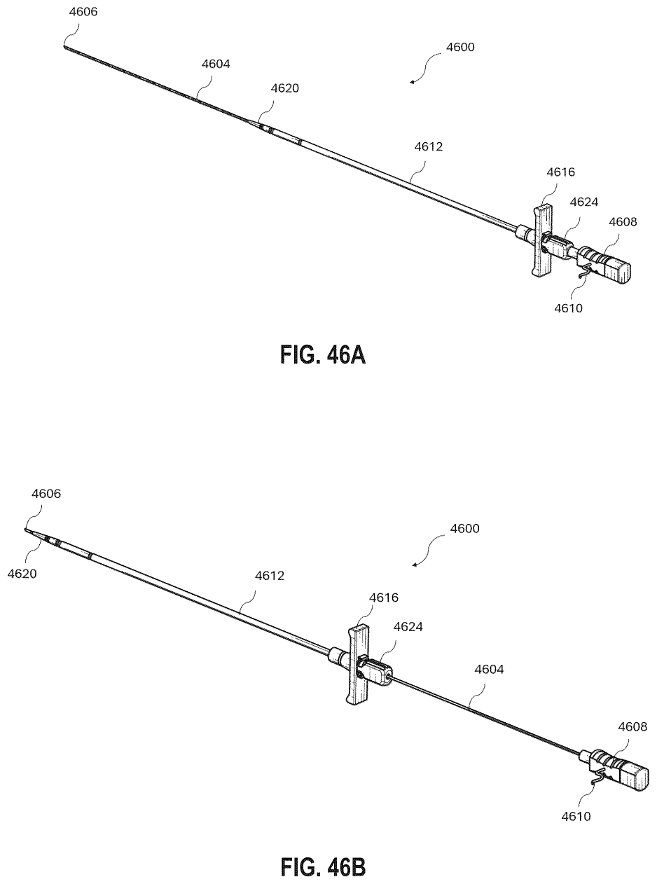

In some embodiments, the introducer assembly may be provided over the stimulation needle assembly prior to insertion of the stimulation needle assembly. For example, the surgeon may receive a stimulation needle and introducer assembly, such as the assembly 4600 shown in FIGS. 46A and 46B. The stimulation needle and introducer assembly 4600 may be pre-assembled. In some embodiments, the stimulation needle and introducer assembly 4600 may be modular and a medical personnel may assemble the components to form the stimulation needle and introducer assembly prior to operation. For example, a distal end of the stimulating needle assembly may be inserted into a proximal end of the introducer assembly prior to insertion of the combined assembly into the patient. In some embodiments, the stimulation needle assembly may be secured to the introducer assembly after insertion of the stimulation needle assembly into the introducer assembly. The securement may be reversible such that the stimulation needle assembly may be removed from the introducer assembly once in position. For example, a hub or handle of the stimulation needle assembly may be reversibly secured to a hub or handle of the introducer assembly.

Referring to FIG. 46A, the stimulation needle and introducer assembly 4600 may comprise a stimulation needle assembly. The stimulation needle assembly may comprise a stimulation needle, and may or may not comprise an insertable stylet. In some embodiments, the stimulation needle assembly may comprise only a stimulation needle, with or without a hub. In some embodiments, the stimulation needle assembly may comprise a stimulation needle and an integrated stylet or a removable stylet. In some cases, the stylet may provide the connection to external stimulation and deliver the stimulation from the stimulation needle assembly, as disclosed elsewhere herein. The stimulation needle may or may not have any of the features disclosed herein with respect to stimulating needles, e.g., stimulating needle 4100 (FIG. 41). The stimulation needle may comprise an elongate needle body 4604 and a needle hub 4608. The stimulation needle may comprise a distal tip 4606. The needle body 4604 may comprise one or more depth markings along the length of the needle body 4604. In some embodiments, the needle body 4604 may not include an inner lumen. The stimulation needle hub 4608 may comprise a connector 4610 to a source of external stimulation. The connector 4610 may be integrated within the needle hub 4608, or may be removably connected, e.g., a J-clip.

Referring to FIG. 46A, the stimulation needle and introducer assembly 4600 may comprise an introducer assembly. The introducer assembly may comprise an introducer sheath 4612 and an introducer handle 4616. The introducer assembly may comprise an obturator disposed within the introducer sheath, the obturator comprising an obturator body 4620 and an obturator handle 4624. The introducer sheath 4612 and/or the obturator body 4620 may be tapered at a distal end. The obturator body 4620 may have an inner diameter larger than an outer diameter of the stimulating needle body to allow the introducer assembly to be advanced over the needle body 4604. The stimulating needle body 4604 may have an outer diameter less than the innermost diameter of the introducer assembly. The stimulating needle body 4604 may have an outer diameter less than the inner diameter of the obturator body 4620, with the difference between the outer diameter of the stimulating needle and the inner diameter of the obturator being less than 0.02 mm, less than 0.03 mm, less than 0.05 mm, less than 0.1 mm, less than 0.15 mm, less than 0.2 mm, less than 0.25 mm, less than 0.3 mm, less than 0.35 mm, less than 0.4 mm, less than 0.5 mm, or a value in a range defined by any of these values. In some embodiments, the introducer assembly may comprise an introducer sheath 4612 and introducer handle 4616, without an obturator. In some embodiments, without an obturator, the stimulating needle assembly may be configured to be sufficiently stiff for the advancement of the introducer assembly. For example, the stimulating needle assembly may have an outer diameter less than the inner diameter of the introducer sheath, with the difference between the outer diameter of the stimulating needle and the inner diameter of the introducer sheath being less than 0.03 mm, 0.05 mm, less than 0.1 mm, less than 0.15 mm, less than 0.2 mm, less than 0.25 mm, less than 0.3 mm, less than 0.35 mm, less than 0.4 mm, less than 0.5 mm, less than 0.6 mm, less than 0.7 mm, or a value in a range defined by any of these values.

Still referring to FIGS. 46A and 46B, the stimulation needle and introducer assembly 4600 may have a longer length than a typical stimulation needle. The elongate needle body 4604 may have a length that is greater than an entire length of the introducer assembly. The length of the needle body 4604 may be greater than the length of the introducer assembly by a length sufficient to be inserted from outside of the individual and extend to a target nerve, e.g., a pudendal nerve. Referring to FIG. 46A, a length of the needle body 4604 extending distally beyond a distal end, e.g., a distal tip, of the introducer assembly, as provided, may be sufficient to reach a target nerve of an individual from any of the approaches described herein, e.g., an ischiorectal approach. In some embodiments, the length of the needle body 4604 extending distally beyond the introducer assembly, as provided, may be sufficient to reach a pudendal nerve, but not a sacral nerve, of the individual. Thus, the introducer assembly may not need to be inserted into the individual when locating the nerve with the stimulation needle assembly of the combined assembly 4600, using the methods described herein. For example, the needle body 4604 may have an entire length greater than an entire length of the introducer assembly by at least 30 mm, at least 40 mm, at least 50 mm, at least 60 mm, at least 70 mm, at least 80 mm, at least 90 mm, at least 100 mm, at least 110 mm, at least 120 mm, at least 130 mm, at least 140 mm, at least 150 mm, and/or less than 200 mm, less than 150 mm, less than 100 mm, less than 80 mm, less than 50 mm, or in a range defined by any of these values. In another example, the length of the needle body 4604 extending distally beyond the introducer assembly, as provided, may be at least 30 mm, at least 40 mm, at least 50 mm, at least 60 mm, at least 70 mm, at least 80 mm, at least 90 mm, at least 100 mm, at least 110 mm, at least 120 mm, at least 130 mm, at least 140 mm, at least 150 mm, and/or less than 200 mm, less than 150 mm, less than 120 mm, less than 100 mm, less than 80 mm, less than 60 mm, or a length in a range defined by any of these values. For example, the needle body 4604 may have a total length of 300 mm or more, and the introducer assembly may have a length of 200 mm or more. Referring to FIG. 46B, the introducer assembly may be advanced over the stimulation needle assembly and into the tissue. The stimulation needle assembly and/or the introducer assembly may comprise one or more radiopaque markers to guide advancement. The stimulation needle assembly may continue to deliver stimulation as the introducer assembly is advanced into the tissue, since the needle remains connected to a source of external stimulation. The continued delivery of stimulation may be used to verify that the distal tip of the stimulation needle assembly remains at a target location as the introducer assembly is advanced into the tissue. Once the introducer assembly is in position, the stimulation needle assembly may be withdrawn from the introducer assembly, and a lead insertion process, as discussed elsewhere herein, may begin.

In some embodiments, as discussed elsewhere herein, a stimulating needle body may have an increased length to allow an introducer assembly to be directly advanced over the stimulating needle body, while still allowing for sufficiently precise handling. The total length of the stimulating needle body may be at least 125 mm, at least 150 mm, at least 175 mm, at least 200 mm, at least 225 mm, at least 250 mm, at least 275 mm, at least 300 mm, and/or less than 300 mm, less than 275 mm, less than 250 mm, less than 200 mm, less than 175 mm, less than 150 mm, or a length in a range defined by any of these values. The stimulating needle body may not have an integrated hub or handle at a proximal end of the stimulating needle, such that the introducer assembly can be advanced over the proximal end of the needle body. However, there may be a difficulty in handling an instrument of increased length without an integrated handle or hub. Additionally, or alternatively, a handle or hub at a proximal end of a long instrument may not be as effective for precise handling during advancement.

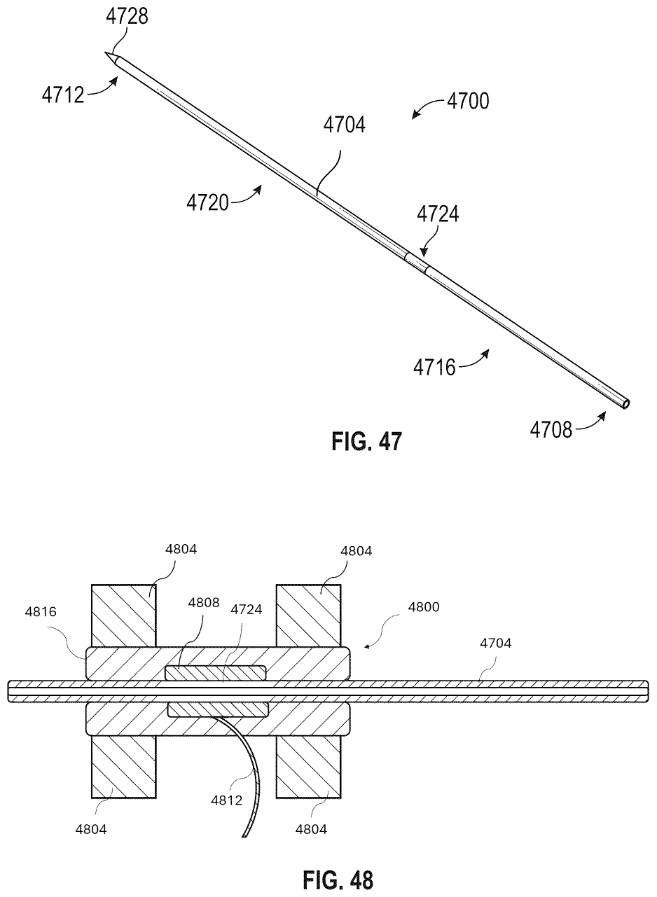

In some embodiments, the stimulating needle may have an outer connection region in a middle portion of the needle body. Referring to FIG. 47, a stimulation needle 4700 may include an needle body 4704. The needle body 4704 may include a proximal end 4708 and a distal end 4712. The needle body 4704 may comprise a proximal insulated region 4716, a distal insulated region 4720, and a connection region 4724. The connection region 4724 may be disposed between the proximal insulated region 4716 and the distal insulated region 4720. The connection region 4724 may be provided anywhere along the length of the needle body configured to remain outside of the patient. For example, the distal insulated region may have a length greater than the distance from the individual's skin to a target nerve. The distal insulated region may have a length greater than a distance from the individual's skin to a pudendal nerve from any of the approaches disclosed elsewhere herein, and/or a length short enough for precise handling. The distal insulated region may have a length of at least 30 mm, at least 40 mm, at least 50 mm, at least 60 mm, at least 70 mm, at least 80 mm, at least 90 mm, at least 100 mm, at least 110 mm, at least 120 mm, at least 130 mm, at least 140 mm, at least 150 mm, and/or less than 150 mm, less than 140 mm, less than 130 mm, less than 120 mm, less than 110 mm, less than 100 mm, less than 90 mm, less than 80 mm, less than 70 mm, less than 60 mm, less than 50 mm, less than 40 mm, or a length in a range defined by any of these values. In some embodiments, the proximal insulated region 4716 may have a length long enough such that the introducer assembly can be safely advanced over the proximal end of the needle body 4704 once the needle is in position. In some embodiments, the proximal insulated region 4716 may have a length long enough such that the introducer assembly may be provided over the proximal insulated region 4716 prior to insertion of the stimulating needle into the individual, and/or short enough to be precisely handled by a surgeon. The length of the proximal insulated region 4716 may be at least 20 mm, at least 30 mm, at least 40 mm, at least 50 mm, at least 60 mm, at least 70 mm, at least 80 mm, at least 90 mm, at least 100 mm, at least 110 mm, at least 120 mm, at least 130 mm, at least 140 mm, at least 150 mm, at least 160 mm, at least 170 mm, at least 180 mm, at least 190 mm, at least 200 mm, at least 210 mm, at least 220 mm, at least 230 mm, at least 240 mm, at least 250 mm, and/or less than 250 mm, less than 240 mm, less than 230 mm, less than 220 mm, less than 210 mm, less than 200 mm, less than 190 mm, less than 180 mm, less than 170 mm, less than 160 mm, less than 150 mm, less than 140 mm, less than 130 mm, less than 120 mm, less than 110 mm, less than 100 mm, less than 90 mm, less than 80 mm, less than 70 mm, less than 60 mm, less than 50 mm, less than 40 mm, less than 30 mm, or a length in a range defined by any of these values. In some embodiments, the needle body 4704 may not include a proximal insulated region and the connection region 4724 may be provided at the proximal end of the needle body 4704. The needle 4700 may comprise an uninsulated stimulating region at or near a distal end 4712 configured to deliver stimulation. For example, the stimulating region may be at a distal tip 4728, or one or more distal electrodes. The outer surface of the needle body 4704 may be uninsulated at the connection region 4724 to allow the needle to conduct stimulation received from a connection to an external stimulator. The connection region 4724 may be configured to interface with a connector providing a connection to an external stimulator. In some embodiments, the connector may comprise a J-clip. In some embodiments, the connector may comprise a handle. In some embodiments, the connector may comprise a removable hub connected to an external source of stimulation, as discussed elsewhere herein.

In some embodiments, a stimulation needle assembly may comprise a needle that may not include a hub or handle at a proximal end of the needle. The lack of a hub or handle may allow for an introducer assembly to be advanced over the proximal end of the needle once it is in position at a target nerve. However, a connection to a source of external stimulation and/or a structure for handling the stimulation needle assembly needle may still be needed. In some embodiments, the stimulating needle assembly may include a removable stylet with a hub and configured to deliver stimulation. The removable stylet may comprise a stylet body and a stylet hub. The stimulating needle may comprise an inner lumen configured to receive the removable stylet. The stimulating needle may include a distal tip. In some embodiments, the distal tip of the needle may be configured to be advanced into the tissue of an individual. In some embodiments, the distal tip of the needle may be rounded, tapered, and/or blunt, as the distal tip of the removable stylet may provide the tissue penetration of the needle assembly. The stimulating needle may be insulated along an entire length of the needle body. The stimulating needle body may have a length less than a length of the stylet body. When the stylet is fully inserted into the needle, a distal end of the stylet may extend distally beyond the distal tip of the needle. The distal end of the stylet extending distally beyond the distal tip of the needle may comprise an uninsulated stimulating region to deliver the stimulation from the external stimulator. Once the stimulation needle assembly is properly placed, the stylet may be removed from the needle, and the introducer assembly may be advanced over the proximal end of the needle, as the needle does not include a hub.

In some embodiments, the stimulating needle assembly may include a removable hub. The removable hub may allow for improved handling of a long instrument, e.g., the stimulating needle. For example, when the needle is over 100 mm long, or any other length disclosed herein, a removable hub may be beneficial for connection at a region that is distal to the proximal end of the needle, e.g., a middle portion of the needle. Additionally, or alternatively, the removable hub may provide a connection between the needle and an external source of stimulation. Additionally, Referring to FIG. 48, the removable hub 4800 may be attached to the stimulating needle body 4704 at a connection region 4724 to interface with an uninsulated portion of the stimulating needle body 4704. The connection region 4724 of the stimulating needle may be arranged anywhere along the stimulating needle that remains external to the patient. For example, the connection region 4724 may be disposed between proximal and distal insulated regions of the stimulating needle, as discussed elsewhere herein. In another example, the connection region 4724 may be at a proximal end of the stimulating needle. The removable hub 4800 may comprise a contact 4808 configured to interface with the connection region 4724 of the stimulating needle. The contact 4808 may be used to deliver stimulation to the needle via a connector 4812, e.g., a lead or wire, from an external source of stimulation. The removable hub 4800 may comprise a hub body 4816. The hub body 4816 may be gripped by a user and provide for handling of the needle during advancement. The removable hub 4816 may comprise a locking mechanism. The locking mechanism may be configured to secure the hub 4800 to the needle body 4704. The locking mechanism may be reversible to allow the hub 4800 to be removed from the needle body 4704 prior to advancement of the introducer assembly. The locking mechanism may be configured to exert at least 15 N, at least 20 N, at least 25 N, at least 30 N, and/or less than 35 N, less than 30 N, less than 25 N, less than 20 N, or a value in a range defined by any of these values, of compressive axial force onto the needle body 4704 to secure the hub 4800 to the needle body 4704 without damaging the needle body. The locking mechanism may comprise one or more lock features 4804. The lock features 4804 may comprise a lock nut, a clip, a latch, etc. Each of the lock features 4804 may be configured to exert at least 2 N, at least 4 N, at least 6 N, at least 8 N, at least 10 N, at least 12 N, at least 15 N, at least 20 N, at least 30 N, and/or less than 35 N, less than 30 N, less than 25 N, less than 20 N, less than 15 N, less than 10 N, less than 8N, less than 6 N, less than 4 N, or a force in a range defined by any of these values, of compressive axial force onto the needle body 4704. In some embodiments, the hub body 4816 comprises a tapered end (not shown in FIG. 48), e.g., a tapered proximal end and/or a tapered distal end. The tapered end may taper towards a longitudinal axis of the needle body 4704. The one or more lock features 4804 may be disposed at a tapered end of the hub body 4816.

In some embodiments, lead placement may comprise a bilateral placement at one or more target nerves, wherein one or more leads are placed at or near both sides of the target nerve. For example, the target nerve(s) may comprise one or more of the pudendal nerve, tibial nerve, peroneal nerve, sacral nerve, cauda equina, pelvic parasympathetic nerves, lumbar sympathetic nerves, and/or nerve branches leading to and from such nerves. Thus, the devices and methods described herein with respect to nerve localization and lead placement may also be used or performed in similar ways on a contralateral side of the individual. The target nerve may comprise the pudendal nerve. In some embodiments, a target nerve may comprise the sacral nerve. In some embodiments, one or more leads may be placed at both the sacral nerve and the pudendal nerve on one or both sides of the body. For example, one or more leads may be placed at the pudendal nerve on one side of the body, and one or more leads may be placed at the sacral nerve on the contralateral side of the body. As another example, one or more leads may be placed at the pudendal nerve on one side of the body, and one or more leads may be placed at the sacral neve on the same side of the body.

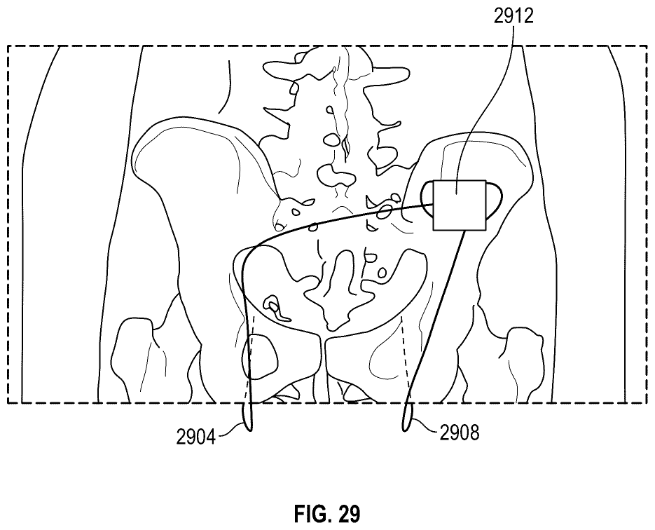

FIG. 29 shows a schematic of a bilateral lead placement at the pudendal nerve. A first lead 2904 may be positioned in the left side of the body at the trunk of the left pudendal nerve. A second lead 2908 may be positioned contralateral to the first lead 2904, in the right side of the body at the trunk of the right pudendal nerve. The first and second leads 2904, 2908 may be tunneled through the gluteal region to connect to a unilateral IPG 2912. The length of the lead contralateral to the IPG may be greater than the length of the lead ipsilateral to the IPG. As illustrated by FIG. 29, the first lead 2904 may be longer than the second lead 2908. For example, the length of the first lead 2904 may be about 550 mm and the length of the second lead 2908 may be about 400 mm. The difference between the length of the first lead 2904 and the second lead 2908 may be between about 100 mm and 200 mm. For example, the difference between the length of two bilateral leads may be about 100 mm, about 110 mm, about 120 mm, about 130 mm, about 140 mm, about 150 mm, about 160 mm, about 170 mm, about 180 mm, about 190 mm, about 200 mm, or a length in a range defined by any of these values.

The low gluteal approach may use surface markings on the skin to traverse the gluteal muscles to reach the ischial spine whereupon electrophysiological responses are used to guide further placement. The ischiorectal approach may enter the skin lateral to the anus near the ischial tuberosity and uses transvaginal or transrectal palpation of the ischial spine +/−electrophysiological responses to place the electrode lead. The low gluteal approach and ischiorectal approach to access the pudendal nerve may benefit from guidance by radiological imaging. It is generally considered that such approaches may target the area of the pudendal nerve at or proximal to the region of Alcock's canal i.e., the nerve trunk. The proximal pudendal nerve trunk may have a fascicular anatomy in which its distal branches are represented as individual fascicles or distinct groups of fascicles. The fascicular anatomy of the pudendal nerve may affect the accuracy of lead placement, where small changes in lead position may favor certain fascicles and thence different motor or afferent effects.

Often, the pudendal nerve may be accessible to electrical stimulation, but the effectiveness of treating incontinence by PNS may be affected by the site of stimulation. Proximal stimulation to the PN trunk (i.e., above Alcock's canal) may provide direct motor stimulation to both urethral and anal sphincters. In some embodiments, proximal stimulation to the PN trunk may lead to some contraction of the pelvic floor/levator ani, based on stimulation being provided proximal to both inferior rectal and perineal nerve branches. In some embodiments, stimulation in the region of Alcock's canal may provide urethral sphincter contraction but less anal sphincter contraction. In some embodiments, the region of Alcock's canal still proximal to the perineal nerve, and some contraction of the pelvic floor/levator ani may be anticipated from stimulation of this region. Unilateral stimulation may lead to bilateral motor effects based on anatomical dissections. More distal stimulation (i.e., of the dorsal genital nerve) may lead to only effects mediated by afferent stimulation. The fascicular anatomy of the PN trunk may be important for accuracy of lead placement.

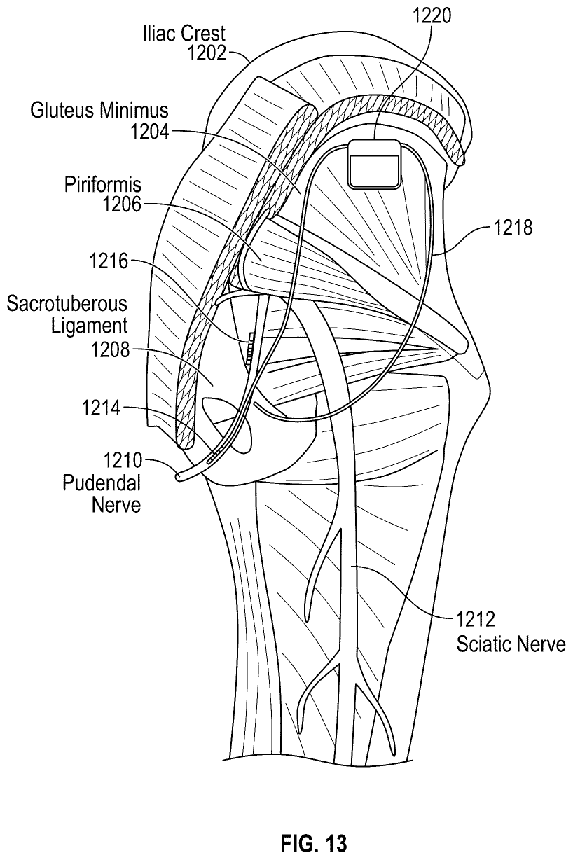

FIGS. 13-16B show the anatomical path desired for lead placements in some embodiments. FIG. 13 shows a schematic of the anatomy and the disposition of leads and IPG in an individual. FIG. 13 shows iliac crest 1202 of the ileum, gluteus minimus 1204, piriformis 1206, sacrotuberous ligament 1208, pudendal nerve 1210, and sciatic nerve 1212. The leads 1214, 1216 may be placed on one or more locations along the length of the pudendal nerve 1210. The wires 1218 of the leads 1214, 1216 may be connected to the IPG 1220. The placement of the leads 1214, 1216 on the pudendal nerve may be verified and fixed before their wires are connected to the IPG.

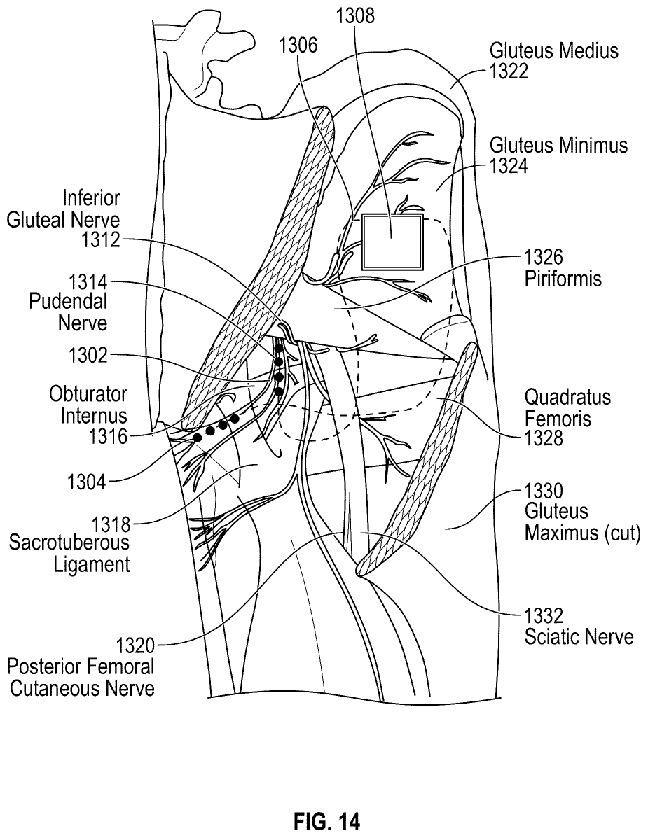

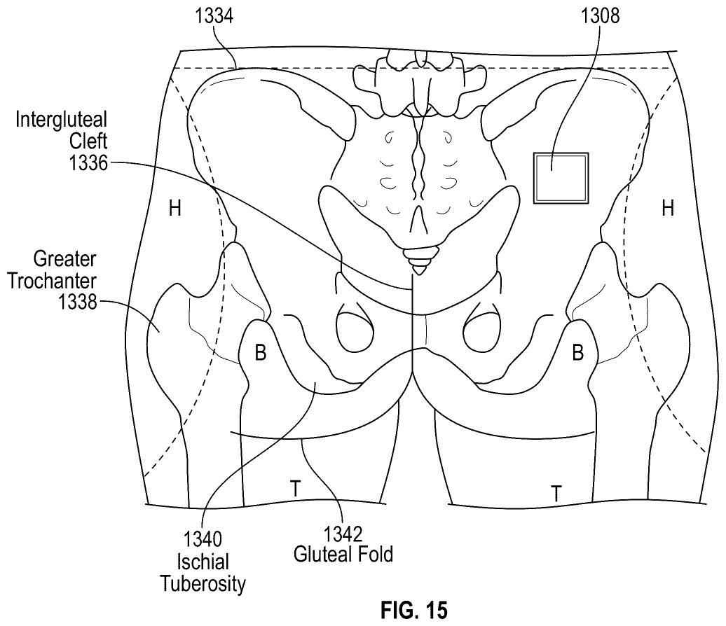

FIGS. 14 and 15 show schematics of the anatomy and implanted leads and IPG in an individual. FIG. 14 shows two leads 1302, 1304, each lead with four electrodes (shown as dark circles), placed on two sections of the pudendal nerve 1314. The wires 1306 of the leads 1302 and 1304 may be connected to the IPG 1308. Shown in FIG. 14 are the inferior gluteal nerve 1312, pudendal nerve 1314, obturator internus 1316, sacrotuberous ligament 1318, posterior femoral cutaneous nerve 1320, gluteus medius 1322, gluteus minimus 1324, piriformis 1326, quadratus femoris 1328, gluteus maximus 1330, and sciatic nerve 1332. FIG. 15 shows the IPG 1308 placed in a pocket in the buttock fat overlying the gluteal muscles. Shown in FIG. 15 are the iliac crest 1334, intergluteal cleft 1336, greater trochanter 1338 of the femur, ischial tuberosity 1340 of the pelvis, and gluteal fold 1342.

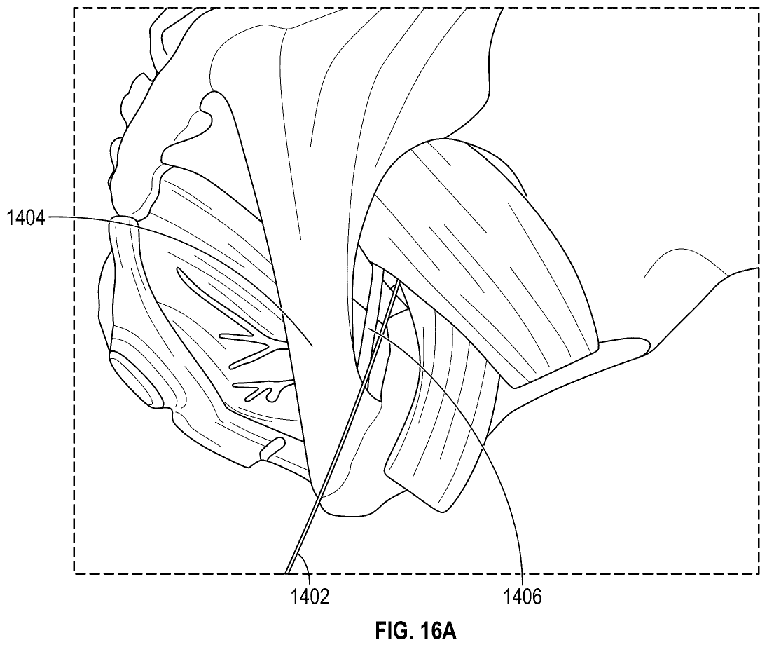

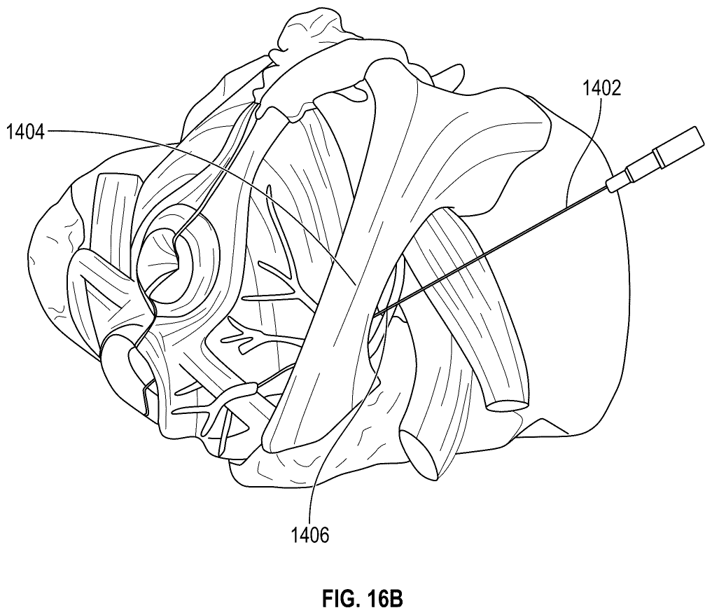

FIGS. 16A and 16B show embodiments of the anatomical paths of the leads using anatomical models. FIG. 16A shows an embodiment of the ischiorectal approach, where a needle 1402, representing the needle of the introducer, is shown passing the sacrotuberous ligament 1404 to access the pudendal nerve 1406. In some embodiments, the needle of the introducer may pierce or pass close to the sacrotuberous ligament 1404 in vivo. FIG. 16B shows embodiment of the low gluteal approach, where the needle 1402 is directed approximating a low gluteal approach to access the pudendal nerve 1406.

### Lead Fixation Devices

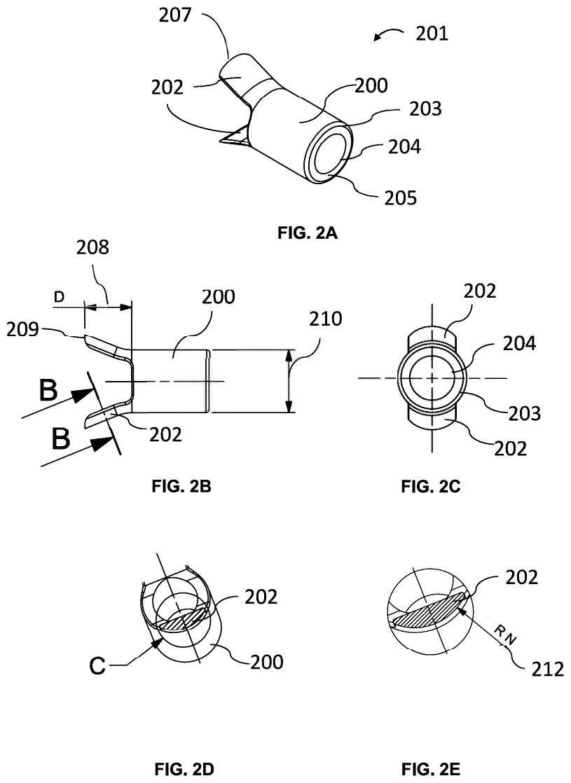

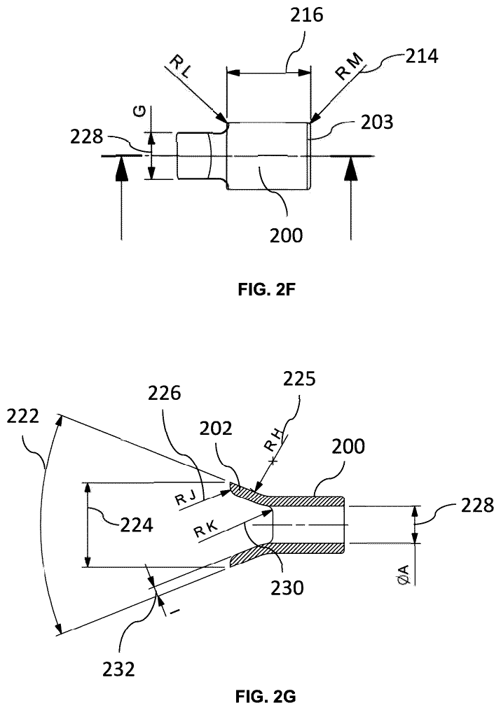

One or more leads (e.g., electrode leads) may be fixed at a target tissue (e.g., a pudendal nerve or tissue adjacent to the pudendal nerve) by a fixation method. For example, one or more leads may be fixed to the tissues surrounding the pudendal nerve. The electrode lead may be fixed at a pudendal nerve or tissue adjacent the pudendal nerve with one or more anchors 201, as shown in FIGS. 2A-2G. The anchors 201 may comprise a body 200 (e.g., a cylindrical body or a collar) that may comprise a lumen 204 configured to receive a lead, as described elsewhere herein. The anchor may comprise a first end 205 and a second end 207. The first end 205 may comprise a rounded and/or filleted edge 203 of a surface of the first end 205. The rounded and/or filleted edge 203 may allow for the anchor 201 to travel into and through an introducer elongated body lumen, as described elsewhere herein, compact or collapse anchor ahead of delivering and/or implanting the anchor and electrode lead. The rounded and/or filleted edge 203 may reduce the frictional force between an inner surface geometry of the introducer elongated body lumen, described elsewhere herein, and the rounded and/or filleted edge 203 of the first end, to collapse or compact the anchor without damaging the anchor or compromising the structural integrity of the anchor. The second end 207 may comprise a free end of one or more barbs 202, as shown in FIG. 2A. The free end of a barb of the one or more barbs may comprise a filleted, chamfered, curved and/or rounded edge 209, as shown in FIG. 2B. The filleted, curved and/or rounded edge 209 may provide a curvature that minimizes frictional forces acted upon e.g., filleted surface of the free end of one or more barbs 202 and an inner surface of an introducer elongated body lumen, described elsewhere herein, as the one or more barbs and electrode lead are translated into and out of the introducer elongated body lumen. Depending on the indication for the one or more leads, one or more parameters of the anchors and/or the barbs described herein may be varied, including but not limited to, material, profile, length, distance to electrodes, angle, number, etc.

The one or more anchors may be made and/or manufactured from a polymer. The polymer may comprise a thermoplastic polyurethane elastomer (TPU). For example, the polymer may comprise thermoplastic polyurethane, i.e., Pellethane™. In some embodiments, the polymer may comprise polytetrafluoroethylene (PTFE). In some embodiments, the anchor may comprise a deformable material.

The one or more anchors may be made from a material with a material stiffness of Shore about 50 D to about 80 D. The shore hardness indicated elsewhere herein by Shore, may comprise a stiffness measured by durometer. A durometer may measure Shore stiffness by determining a penetration of a durometer indenter foot into a sample test. The one or more anchors may be made from a material with a material stiffness of Shore about 40 D to about 90 D, about 50 D to about 80 D, about 50 D to about 70 D, or about 60 D to 70 D. The one or more anchors may be made from a material with a material stiffness of Shore about 50 D, about 52 D, about 54 D, about 56 D, about 58 D, about 60 D, about 65 D, about 70 D, about 75 D, or about 80 D. The one or more anchors may be made from a material with a material stiffness of Shore at least about 50 D, about 52 D, about 54 D, about 56 D, about 58 D, about 60 D, about 65 D, about 70 D, or about 75 D. In some cases, the one or more anchors may be made from a material with a material stiffness of Shore at most about 52 D, about 54 D, about 56 D, about 58 D, about 60 D, about 65 D, about 70 D, about 75 D, or about 80 D.

The rounded and/or filleted edge 203 of the body 200, may comprise a radius 214 of about 0.01 mm to about 0.3 mm. For example, the rounded and/or filleted edge 203 of the body 200, may comprise a radius 214 of about 0.08 mm to about 0.3 mm, or about 0.1 mm to about 0.3 mm. In some cases, the rounded and/or filleted edge 203 of the body 200, may comprise a radius 214 of about 0.01 mm, about 0.05 mm, about 0.08 mm, about 0.1 mm, about 0.15 mm, about 0.2 mm, or about 0.3 mm. In some cases, the rounded and/or filleted edge 203 of the body 200, may comprise a radius 214 of at least about 0.01 mm, about 0.05 mm, about 0.08 mm, about 0.1 mm, about 0.15 mm, or about 0.2 mm. In some cases, the rounded and/or filleted edge 203 of the body 200, may comprise a radius 214 of at most about 0.05 mm, about 0.08 mm, about 0.1 mm, about 0.15 mm, about 0.2 mm, or about 0.3 mm. In some cases, the body 200 may be tapered in a similar manner as the introducer to facilitate introduction of the anchor into the introducer. In some cases, the body 200 may comprise a deformable material shaped like a donut.

The body 200 of the anchor may comprise a length 216 of about 0.5 mm to about 6 mm. For example, the body 200 of the anchor may comprise a length 216 of about 1 mm to about 6 mm, about 2 mm to about 6 mm, or about 3 mm to about 6 mm. The body 200 of the anchor may comprise a length 216 of about 0.5 mm, about 1 mm, about 1.5 mm, about 2 mm, about 2.5 mm, about 3 mm, about 4 mm, about 5 mm, or about 6 mm. In some cases, the body 200 of the anchor may comprise a length 216 of at least about 0.5 mm, about 1 mm, about 1.5 mm, about 2 mm, about 2.5 mm, about 3 mm, about 4 mm, or about 5 mm. In some cases, the body 200 of the anchor may comprise a length 216 of at most about 1 mm, about 1.5 mm, about 2 mm, about 2.5 mm, about 3 mm, about 4 mm, about 5 mm, or about 6 mm.

The body 200 of the anchor may comprise an outer diameter 210 of about 0.5 mm to about 6 mm. The outer diameter may comprise a diameter of a circular cross section of the body 200 of the anchor. The body 200 of the anchor may comprise an outer diameter 210 of about 1 mm to about 6 mm, about 2 mm to about 6 mm, or about 3 mm to about 6 mm. The body 200 of the anchor may comprise an outer diameter 210 of about 0.5 mm, about 1 mm, about 1.5 mm, about 2 mm, about 2.5 mm, about 3 mm, about 4 mm, about 5 mm, or about 6 mm. In some cases, the body 200 of the anchor may comprise an outer diameter 210 of at least about 0.5 mm, about 1 mm, about 1.5 mm, about 2 mm, about 2.5 mm, about 3 mm, about 4 mm, or about 5 mm. In some cases, the body 200 of the anchor may comprise an outer diameter 210 of at most about 1 mm, about 1.5 mm, about 2 mm, about 2.5 mm, about 3 mm, about 4 mm, about 5 mm, or about 6 mm.

The body 200 of the anchor may comprise an inner diameter 228 of about 0.5 mm to about 6 mm. For example, the body 200 of the anchor may comprise an inner diameter 228 of about 1 mm to about 6 mm, about 2 mm to about 6 mm, or about 3 mm to about 6 mm. The body 200 of the anchor may comprise an inner diameter 228 of about 0.5 mm, about 1 mm, about 1.5 mm, about 2 mm, about 2.5 mm, about 3 mm, about 4 mm, about 5 mm, or about 6 mm. In some cases, the body 200 of the anchor may comprise an inner diameter 228 of at least about 0.5 mm, about 1 mm, about 1.5 mm, about 2 mm, about 2.5 mm, about 3 mm, about 4 mm, or about 5 mm. In some cases, the body 200 of the anchor may comprise an inner diameter 228 of at most about 1 mm, about 1.5 mm, about 2 mm, about 2.5 mm, about 3 mm, about 4 mm, about 5 mm, or about 6 mm.

The anchor 201 may comprise one or more barbs 202 e.g., a pair of barbs or two or more barbs. In some cases, the anchor may comprise two or more barbs. The two or more barbs of the anchor may comprise 2, 3, 4, 5, 6, 7, 8, 9, or 10 barbs. The barb 202 of the anchor may be made of the same material as the body 200 (e.g., a unibody material). The unibody material may comprise a material of injection molded plastic. By manufacturing the anchor body and one or more barbs from the same material, the cost of the anchors may be significantly reduced. The barbs 202 may be radially symmetric around the body of anchor 200. In some instances, two or more barbs of the anchor may not be radially symmetric around the body of the anchor 200. Two or more barbs of the anchor may be equally spaced along a circumference of a cross section of the anchor body (e.g., equally spaced at a rotation angle). Each of the two or more barbs 202 may be configured to extend along a radius of a circular cross-section of the body of the anchor 200.

The one or more barbs 202 may comprise thermally set barbs. Thermally set barbs may comprise a material configured to maintain, hold, and/or fix a geometry and/or shape of a barb when exposed to a temperature or temperature range. The thermally set barbs may comprise a shape memory polymer material configured to maintain a shape and/or geometry, as described elsewhere herein, when exposed to a temperature or temperature range. The shape memory polymer material may comprise thermoplastics, (meth)acrylates, polyurethanes, blends of polyurethane and polyvinylchloride, or any combination thereof.

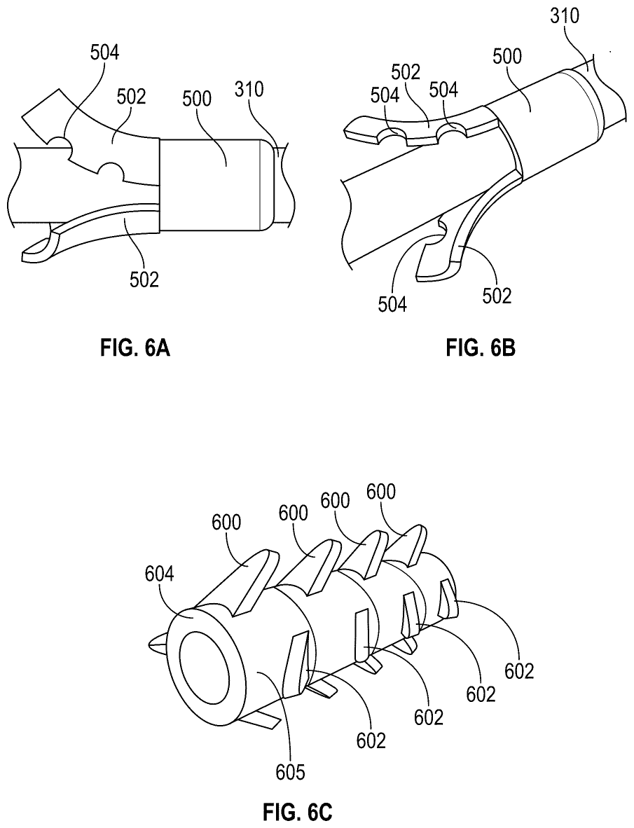

The barb 202 may comprise a rectangular, oval, or triangular profile. For example, a rectangular profile of the barb 202 is shown in FIGS. 2A-2C. In some cases, the barb (600, 602), may comprise a triangular profile, as shown in FIG. 6C. A first one or more triangular profile barb(s) 600 may be coupled to a first body segment 604, and a second one or more triangular profile barb(s) 602 may be positioned on a second body segment 602. The first 604 and second 605 body segments may be coupled to one another to form an anchor body. In some instances, the barb of the two or more barbs of an anchor may comprise a serrated barb 502, where the serrated barb comprises a profile with one or more cut features 504 or protrusions, as shown in FIGS. 6A and 6B. The one or more cut features may comprise a circular geometry. The circular geometry may comprise a diameter of about 0.25 mm to about 0.5 mm. The serrated barb 502 may be mechanically coupled and/or fixed to an anchor body 500. The serrated barb 502 and associated anchor body 500 may comprise dimension, as described elsewhere herein for barbs and anchor bodies. In some cases, a surface of the serrated barb 502 may increase friction between the surface of the serrated barb 502 a surface of tissue surrounding the serrated barb 502. The increase in friction may maintain and/or fix a position of an electrode lead to which the serrated barb 502 is coupled to.

The one or more barbs of the anchor 201 may comprise a length 208 of about 0.2 mm to about 5 mm. The one or more barbs of the anchor 201 may comprise a length 208 of about 0.5 mm to about 5 mm, about 1 mm to about 5 mm, or about 2 mm to about 5 mm. The one or more barbs of the anchor 201 may comprise a length 208 of about 0.2 mm, about 0.5 mm, about 0.8 mm, about 1 mm, about 1.5 mm, about 2 mm, about 3 mm, about 4 mm, or about 5 mm. The one or more barbs of the anchor 201 may comprise a length 208 of at least about 0.2 mm, about 0.5 mm, about 0.8 mm, about 1 mm, about 1.5 mm, about 2 mm, about 3 mm, or about 4 mm. The one or more barbs of the anchor 201 may comprise a length 208 of at most about 0.5 mm, about 0.8 mm, about 1 mm, about 1.5 mm, about 2 mm, about 3 mm, about 4 mm, or about 5 mm.

A cross-section of the barb 202, as shown in FIGS. 2B, 2D, and 2E may comprise a radius of curvature 212. The radius of curvature 212 may comprise a radius of about 0.5 mm to about 3 mm. The radius of curvature 212 may comprise a radius of about 1 mm to about 3 mm, or about 1.5 mm to about 3 mm. The radius of curvature 212 may comprise a radius of about 0.5 mm, about 1 mm, about 1.5 mm, about 2 mm, about 2.5 mm, or about 3 mm. In some cases, the radius of curvature 212 may comprise a radius of at least about 0.5 mm, about 1 mm, about 1.5 mm, about 2 mm, or about 2.5 mm. In some cases, the radius of curvature 212 may comprise a radius of at most about 1 mm, about 1.5 mm, about 2 mm, about 2.5 mm, or about 3 mm.

The barb 202 may comprise a thickness 232 of about 0.1 mm to about 1.5 mm. For example, the barb 202 may comprise a thickness 232 of about 0.2 mm to about 1.5 mm, or about 0.3 mm to about 1.5 mm. The barb 202 may comprise a thickness 232 of about 0.1 mm, about 0.2 mm, about 0.3 mm, about 0.35 mm, about 0.4 mm, about 0.5 mm, about 0.7 mm, about 1 mm, or about 1.5 mm. In some cases, the barb 202 may comprise a thickness 232 of at least about 0.1 mm, about 0.2 mm, about 0.3 mm, about 0.35 mm, about 0.4 mm, about 0.5 mm, about 0.7 mm, or about 1 mm. In some cases, the barb 202 may comprise a thickness 232 of at most about 0.2 mm, about 0.3 mm, about 0.35 mm, about 0.4 mm, about 0.5 mm, about 0.7 mm, about 1 mm, or about 1.5 mm.

The barb 202 when in a deployed, expanded, and/or extended state may form a curved surface 225 between the barb 202 and body 200 of the anchor. The curved surface 225 may comprise a radius of about 0.5 mm to about 2 mm. For example, the curved surface 225 may comprise a radius of about 0.7 mm to about 2 mm, or about 1 mm to about 2 mm. The curved surface 225 may comprise a radius of about 0.5 mm, about 0.7 mm, about 1 mm, about 1.1 mm, about 1.25 mm, about 1.3 mm, about 1.4 mm, about 1.5 mm, or about 2 mm. In some instances, the curved surface 225 may comprise a radius of at least about 0.5 mm, about 0.7 mm, about 1 mm, about 1.1 mm, about 1.25 mm, about 1.3 mm, about 1.4 mm, or about 1.5 mm. In some instances, the curved surface 225 may comprise a radius of at most about 0.7 mm, about 1 mm, about 1.1 mm, about 1.25 mm, about 1.3 mm, about 1.4 mm, about 1.5 mm, or about 2 mm.

The barb 202 when in a deployed, expanded, and/or extended state may form an internal curved surface 230 between the barb 202 and the body 200 of the anchor. The internal curved surface 230 comprises a radius of about 0.01 mm to about 0.4 mm. For example, the internal curved surface 230 comprises a radius of about 0.05 mm to about 0.4 mm, or about 0.2 mm to about 0.4 mm. The internal curved surface 230 comprises a radius of about 0.01 mm, about 0.05 mm, about 0.1 mm, about 0.15 mm, about 0.2 mm, about 0.25 mm, about 0.3 mm, or about 0.4 mm. In some cases, the internal curved surface 230 comprises a radius of at least about 0.01 mm, about 0.05 mm, about 0.1 mm, about 0.15 mm, about 0.2 mm, about 0.25 mm, or about 0.3 mm. In some cases, the internal curved surface 230 comprises a radius of at most about 0.05 mm, about 0.1 mm, about 0.15 mm, about 0.2 mm, about 0.25 mm, about 0.3 mm, or about 0.4 mm.

The second or free end 207 of a barb 202 may comprise a filleted, curved and/or rounded edge 209 with a radius 226 of about 0.01 mm to about 0.4 mm. For example, the free end 207 of a barb 202 may comprise a filleted, curved and/or rounded edge 209 with a radius 226 of about 0.05 mm to about 0.4 mm, or about 0.1 mm to about 0.4 mm. The free end 207 of a barb 202 may comprise a filleted, curved and/or rounded edge 209 with a radius 226 of about 0.01 mm, about 0.05 mm, about 0.1 mm, about 0.15 mm, about 0.2 mm, about 0.25 mm, about 0.3 mm, or about 0.4 mm. In some cases, the free end 207 of a barb 202 may comprise a filleted, curved and/or rounded edge 209 with a radius 226 of at least about 0.01 mm, about 0.05 mm, about 0.1 mm, about 0.15 mm, about 0.2 mm, about 0.25 mm, or about 0.3 mm. In some cases, the free end 207 of a barb 202 may comprise a filleted, curved and/or rounded edge 209 with a radius 226 of at most about 0.05 mm, about 0.1 mm, about 0.15 mm, about 0.2 mm, about 0.25 mm, about 0.3 mm, or about 0.4 mm.

When two or more of the barbs 202 are in an extended, deployed, and/or expanded state, as shown in FIGS. 2A-2C, and 2G, they may comprise a distance 224 between an outer surface of a first barb and an outer surface of a second barb of the two or more barbs 202. The distance 224 may comprise about 1 mm to about 5 mm. For example, the distance 224 may comprise about 2.5 mm to about 5 mm, or 3 mm to about 5 mm. The distance 224 may comprise about 1 mm, about 2.5 mm, about 3 mm, about 3.5 mm, about 4 mm, about 4.5 mm, or about 5 mm. In some cases, the distance 224 may comprise at least about 1 mm, about 2.5 mm, about 3 mm, about 3.5 mm, about 4 mm, or about 4.5 mm. In some cases, the distance 224 may comprise at most about 2.5 mm, about 3 mm, about 3.5 mm, about 4 mm, about 4.5 mm, or about 5 mm.

When two or more of the barbs 202 are in an extended, deployed, and/or expanded state, as shown in FIGS. 2A-2C, and 2G, they may form an angle 222 between an outer surface of a first barb and an outer surface of a second barb of the two or more barbs 202. The angle 222 may comprise about 20 degrees to about 180 degrees. For example, the angle 222 may comprise about 30 degrees to about 180 degrees, about 40 degrees to about 180 degrees, or about 50 degrees to about 180 degrees. The angle 222 may comprise about 20 degrees, about 25 degrees, about 30 degrees, about 40 degrees, about 50 degrees, about 60 degrees, about 70 degrees, about 80 degrees, about 90 degrees, about 120 degrees, about 160 degrees, or about 180 degrees. In some cases, the angle 222 may comprise at least about 20 degrees, about 25 degrees, about 30 degrees, about 40 degrees, about 50 degrees, about 60 degrees, about 70 degrees, about 80 degrees, about 90 degrees, about 120 degrees, or about 160 degrees. In some cases, the angle 222 may comprise at most about 25 degrees, about 30 degrees, about 40 degrees, about 50 degrees, about 60 degrees, about 70 degrees, about 80 degrees, about 90 degrees, about 120 degrees, about 160 degrees, or about 180 degrees.

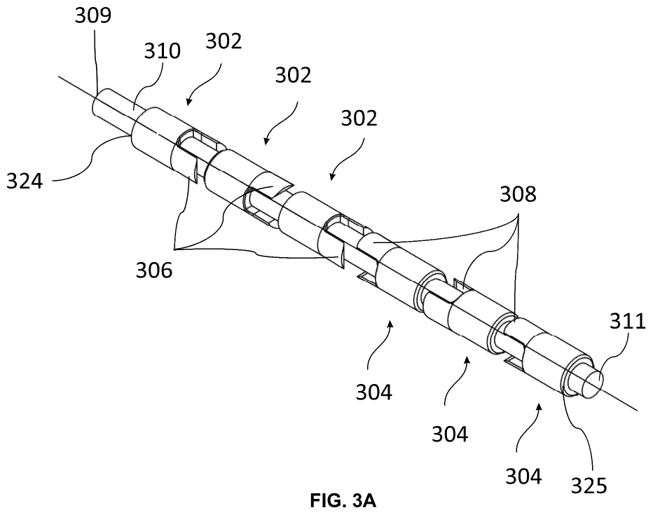

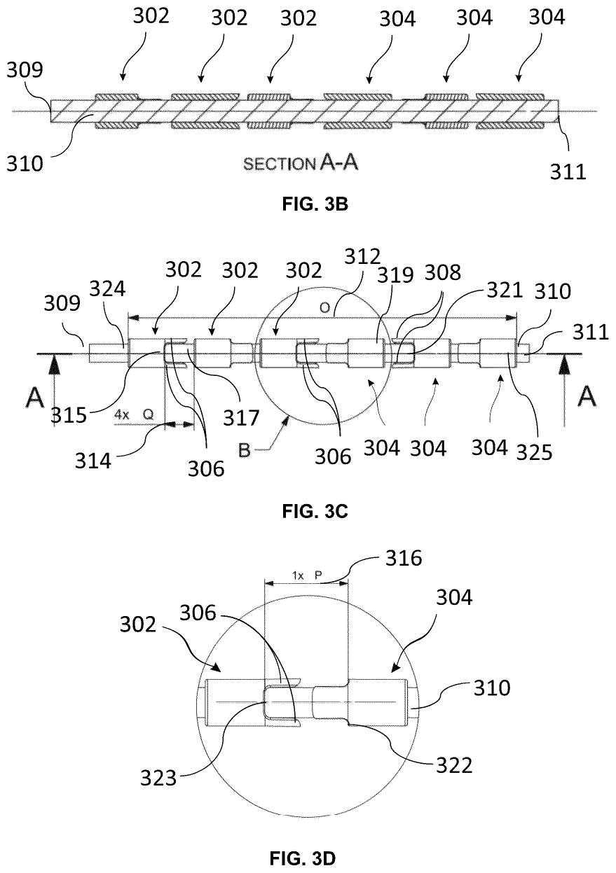

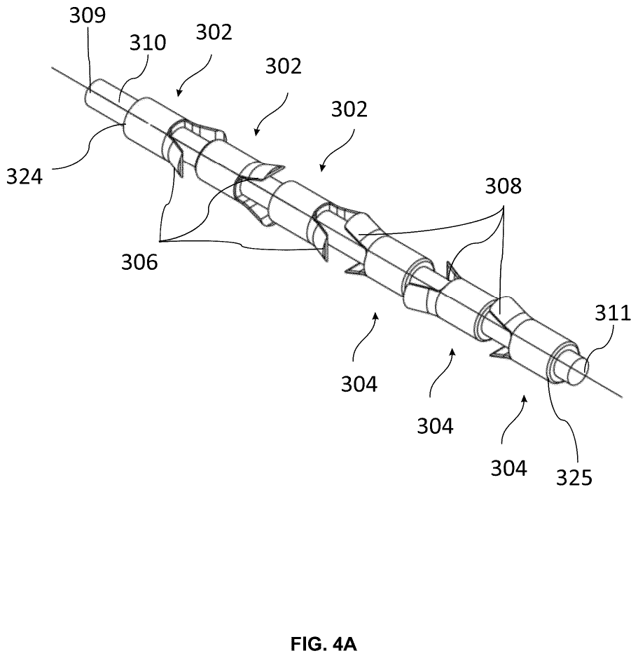

FIGS. 3A-3D illustrate an electrode lead device having a lead 310 and one or more anchors (302, 304). The lead 310 can include one or more electrodes. In some embodiments, the stimulation electrodes may be located near a distal end of the lead. Each of the one or more anchors (302, 304) may be positioned proximal of each of the one or more stimulation electrodes that is configured to contact or apply stimulation to the tissue. Although, in other configurations, the one or more anchors may be positioned elsewhere, for example, between electrodes.

As illustrated, each anchor (302, 304) includes a collar with one or more barbs (e.g., two, three, four, or more) extending from an end of the collar at an oblique angle relative to a longitudinal axis of the lead body. The barbs (306, 308) may only extend from one end of the collar. All of the barbs (306, 308) on an individual anchor may extend in the same axial direction. The barbs (306, 308) may be collapsible for introduction into the body. In some configurations, the barbs (306, 308) may all collapse in the same direction such that the barbs (306, 308) are all oriented in the same direction for delivery. The anchors (302, 304) may be arranged such that the barbs (306, 308) on adjacent anchors (302, 304) are circumferentially offset. For example, the barbs on a first anchor may be circumferentially offset from the barbs on a second, adjacent anchor by 90 degrees. The barbs on the first anchor can be entirely offset from the barbs on the second anchor. For example, where the anchors have only two barbs, the barbs on the first anchor will be entirely offset from the barbs on the second anchor. In other configurations, the barbs may be circumferentially offset by 30 degrees, 45 degrees, or 60 degrees. The barbs on the first anchor may extend in a same or different axial direction than the barbs on the second anchors. The anchors (302, 304) may be equidistant from each other.

As illustrated, there are a plurality of anchors (302, 304) with bi-directional orientation. A first set of anchors 302 may be oriented in a first direction and a second set of anchors 304 may be oriented in a second direction, different or opposite from the first direction. Together, the first set of anchors 302 and the second set of anchors 304 account for all of the plurality of anchors (302, 304). For example, the first set of anchors 302 may be oriented in a proximal or distal direction, while the second set of anchors 304 oriented in the other one of the proximal or distal direction. With the bi-directional configuration, the barbs 306 on the first set of anchors 302 may extend in the first direction, while the barbs 308 on the second set of anchors 304 extend in the second direction. In other words, the barbs 306 on the first set of anchors 302 may extend in the opposite direction from the barbs 308 on the second set of anchors 304. As illustrated, the first set of anchors 304 may be arranged adjacent to each other, and the second set of anchors 306 may be arranged adjacent to each other. The first set of anchors 302 may be positioned entirely between the stimulation electrodes and the second set of anchors 304. The barbs 306 on the first set of anchors 302 may extend in a proximal direction, and the barbs 308 on the second set of anchors 304 may extend in a distal direction. The barbs 306 on the first set of anchors 302 may extend toward the second set of anchors 304, and the barbs 308 on the second set of anchors 304 may extend toward the first set of anchors 302. The number of anchors in each of the first set of anchors 302 and the second set of anchors 304 may be the same or different. For example, the overall arrangement of anchors (302, 304) may be asymmetrical along a length of the lead 310. In other embodiments described below, a single anchor (302, 304) may incorporate bi-directional barbs (306, 308).

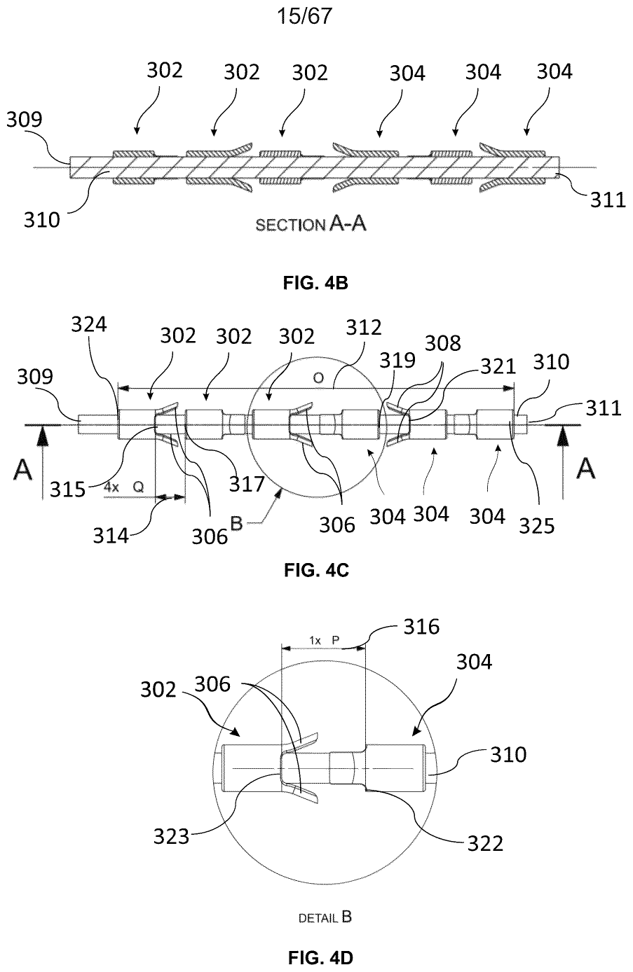

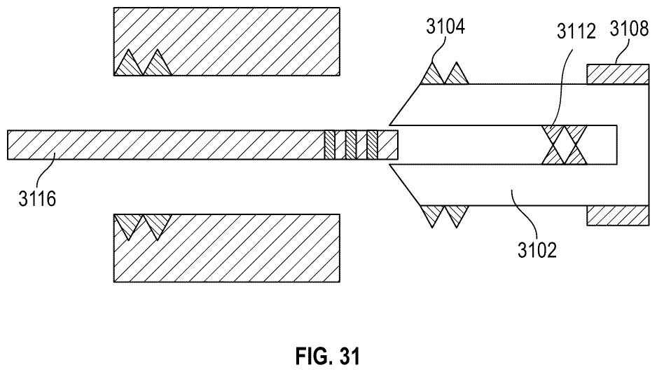

One or more anchors (302, 304, 306, 608) may be coupled to a lead 310 (e.g., an electrode lead), as shown in FIGS. 3A-3D and FIGS. 4A-4D. Depending on the indication, the number of anchors used for fixation of an electrode lead may be varied. For example, four anchors may be couple to the lead 310, rather than the six anchors illustrated in FIGS. 3A-3D and FIGS. 4A-4D. The one or more anchors (302, 304, 306, 308), may comprise an anchor body (302, 304) and one or more barbs (306, 308), as described elsewhere herein. The one or more anchors (302, 304, 306, 308) may be fixed in position on the lead 310. The one or more anchors may be fixed to the lead by adhesion. In some cases, the one or more anchors (302, 304, 306, 308) may be removably coupled to the lead 310. The one or more anchors may comprise one or more mating features on the inner surface of the anchor body, as shown in FIG. 31. The mating features 3112 of the anchor 3102 may be configured to mate with a corresponding mating feature of the lead 3116. For example, the inner surface of the body of each of the one or more anchors may comprise ridges or threads configured to mate with corresponding grooves or thread holes of the lead body. The anchor 3102 may comprise one or more gripping features 3108 configured to be gripped by fingers or a tool during attachment of the anchor 3102 to the lead 3116. In some cases, the anchor 3102 may comprise one or more teeth 3104 disposed on the outer surface of the anchor configured to grip to bone and/or tissue. The electrode lead body may include one or more stoppers at either or both ends of the array of anchors or therebetween. The stopper(s) may ensure the anchors do not migrate or slip off the lead during revision or other high axial forces. The stopper may be a tubular body, for example made of pellethane. The tubular body may be longer than one of the anchors. For example, the tubular body may have a length of at least 3 mm. The tubular body may be at least 0.5 longer than any one of the anchors.

The one or more anchors (302, 304, 306, 608) may comprise a first orientation or a second orientation. A first orientation of one or more anchors (302, 306) may comprise two or more barbs 306 that expand, as shown in FIG. 4A, towards a proximal end 311 of the lead 310. A second orientation of one or more anchors (304, 308) may comprise two or more barbs 308, as shown in FIG. 4A, that expand towards a distal end 309 of the lead 310. The proximal orientation of the expanded and/or extended two or more barbs of the first orientation with respect to the distal orientation of the expanded two or more barbs in the second orientation may provide a beneficial effect of stabilizing and fixing a position of the lead 310 at a target implanted tissue or anatomical feature (e.g., a pudendal nerve). The opposing orientation of the two or more barbs of a first orientation and a second orientation may maintain and/or fixes the position of the lead despite any force (e.g., a pushing, compressive, or tensile force) applied to the implanted electrode lead. The one or more anchors and/or barbs of opposing orientations may fix the position of the electrode lead against applied forces going inwards, outwards, and/or laterally. The one or more anchors and/or barbs of opposing orientations may provide stability in at least three different directions to prevent ingress, egress, and lateralization. As described elsewhere herein, such a benefit conferred by the orientation of the two orientations of anchor may improve the targeted treatment for a subject with the implanted lead by reducing lead migration relative to the tissue as the subject contracts and flexes muscles, moves, or during active loading (e.g., exercise) or passive loading (e.g., sitting or sleeping) of the region of their body where the lead is implanted thus improving robustness and efficacy of treating incontinence with electrical stimulation.

One or more anchors of a first orientation may be provided adjacent and/or spaced at any number of anchors away from the one or more anchors of a second orientation on a lead. For example, two anchors of a first orientation may be provided adjacent to one anchor of the second orientation on a lead, or two anchors of a second orientation may be provided adjacent to one anchor of the first orientation on the lead.

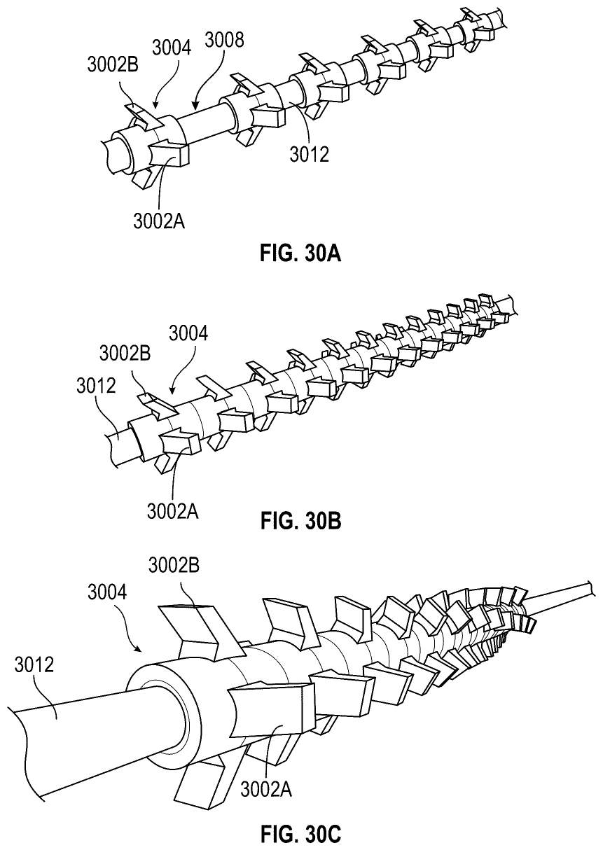

The one or more anchors in an anchor assembly of a lead may each comprise a separate collar with one or more barbs. The one or more barbs may extend from a portion of the collar of the anchor. As shown in FIGS. 30A-C, the one or more barbs may extend from a middle portion of the collar of each anchor. In some embodiments, the one or more barbs may extend from one or both ends of the collar. As shown in FIG. 30A, the one or more anchors 3004 may be spaced apart from each other by a distance 3008 along the length of the lead 3012. The distance 3008 may comprise a distance of about 1 mm to 20 mm. For example, the distance may comprise about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, or a distance in a range defined by any of the preceding values. In some cases, the distance may comprise less than 1 mm. As shown in FIG. 30B, the one or more anchors 3004 may be disposed immediately adjacent to each other, such that they are joined into a single unit. In some embodiments, the collar of each anchor 3004 may comprise one or more mating features such that a collar may interlock with an adjacent collar having a corresponding mating feature. The mating features may comprise a rectangular cut-out or a similar inter-digitating pattern. The one or more barbs on an individual anchor 3004 may extend in different axial directions. For example, the barbs on an individual anchor 3004 may have a bi-directional orientation. A first set of barbs 3002A on a single anchor 3004 may be oriented in a first direction, and a second set of barbs 3002B on the same anchor may be oriented in a second direction, different or opposite from the first direction. For example, the first set of barbs 3002A may be oriented in a proximal or distal direction, while the second set of barbs 3002B may be oriented in the other one of the proximal or distal direction. The one or more barbs may be arranged on the collar such that each barb is only adjacent to a barb of a different direction. For example, the anchor 3004 has one or more barbs alternating in direction along the circumference of the collar. In some embodiments, the barbs may be arranged such that two barbs of the same direction are adjacent each other. For example, an anchor may have a first set of barbs of one direction on one half of the collar, and a second set of barbs of a second direction on the other half of the collar. The barbs may be arranged as suitable for the particular human anatomy in which they will be deployed. The one or more anchors 3004 and bi-directional barbs 3002A, 3002B may share characteristics of any of the anchor and barb embodiments described herein, including but not limited to, material, angle, profile, length, distance to electrodes, number, etc.

The one or more anchors (302, 304, 306, 608) may comprise a length 312 measured from a surface of the distal most anchor body 302 to a surface of the proximal most anchor body 304, as shown in FIGS. 3C and 4C. The length 312 may comprise a distance of about 20 mm to about 50 mm. For example, the length 312 may comprise a distance of about 24 mm to about 50 mm, or about 30 mm to about 50 mm. The length 312 may comprise a distance of about 20 mm, about 22 mm, about 24 mm, about 26 mm, about 28 mm, about 30 mm, about 40 mm, or about 50 mm. In some cases, the length 312 may comprise a distance of at least about 20 mm, about 22 mm, about 24 mm, about 26 mm, about 28 mm, about 30 mm, or about 40 mm. In some cases, the length 312 may comprise a distance of at most about 22 mm, about 24 mm, about 26 mm, about 28 mm, about 30 mm, about 40 mm, or about 50 mm.

Two or more barbs 306 of each of one or more anchors (302, 306) of a first orientation or one or more anchors (304, 308) of a second orientation may be positioned at a rotation angle from each other, as shown in FIG. 3A and FIG. 4A. The rotation angle may comprise an angle of about 1 degree to about 180 degrees. For example, the rotation angle may comprise an angle of about 5 degrees to about 180 degrees, about 20 degrees to about 180 degrees, or about 30 degrees to about 180 degrees. The rotation angle may comprise an angle of about 1 degree, about 5 degrees, about 10 degrees, about 20 degrees, about 30 degrees, about 40 degrees, about 50 degrees, about 90 degrees, about 120 degrees, about 140 degrees, or about 180 degrees. In some cases, the rotation angle may comprise an angle of at least about 1 degree, about 5 degrees, about 10 degrees, about 20 degrees, about 30 degrees, about 40 degrees, about 50 degrees, about 90 degrees, about 120 degrees, or about 140 degrees. In some cases, the rotation angle may comprise an angle of at most about 5 degrees, about 10 degrees, about 20 degrees, about 30 degrees, about 40 degrees, about 50 degrees, about 90 degrees, about 120 degrees, about 140 degrees, or about 180 degrees.

Two or more barbs of adjacent anchors may comprise at least about a 90-degree angular rotation from one another. In some cases, two or more barbs of adjacent anchors may comprise less than a 90-degree angular rotation from one another. In some cases, as shown in FIG. 30C, the barbs on the anchors 3004 may form a spiral pattern which may facilitate burying into the tissue.

A first anchor and a second anchor of the first orientation or the second orientation may be provided at a distance 314 on the lead 310, as shown in FIGS. 3C and 4C. For a first anchor and a second anchor of the first orientation (302, 306), the distance 314 may be measured from a proximal surface 315 of the anchor body 200 of a first anchor to distal surface 317 of the second anchor. In some cases, a first anchor and a second anchor of the second orientation (304, 308), the distance may be measured from a proximal surface 319 of the anchor body 200 of a first anchor to distal surface 321 of the second anchor. The distance 314 may comprise a length of about 1.8 mm to about 5 mm. For example, the distance 314 may comprise a length of about 2 mm to about 5 mm, or about 3 mm to about 5 mm. In some cases, the distance 314 may comprise a length of at least about 1.8 mm, about 2 mm, about 2.1 mm, about 2.2 mm, about 2.4 mm, about 2.5 mm, about 3 mm, or about 4 mm. In some cases, the distance 314 may comprise a length of at most about 2 mm, about 2.1 mm, about 2.2 mm, about 2.4 mm, about 2.5 mm, about 3 mm, about 4 mm, or about 5 mm.

A first anchor (302, 306) of the first orientation may be provided at a distance 316 from a second anchor (304, 310) of the second orientation, as shown in FIGS. 3D and 4D. The distance 316 may be measured from a proximal surface 316 of the first anchor in the first orientation to a distal surface 322 of the second anchor in the second orientation. The distance 316 may comprise a length of about 2.5 mm to about 5 mm. For example, the distance 316 may comprise a length of 3 mm to about 5, or about 4 mm to about 5 mm. The distance 316 may comprise a length of about 2.5 mm, about 3 mm, about 3.1 mm, about 3.2 mm, about 3.4 mm, about 3.5 mm, about 4 mm, or about 5 mm. In some instances, the distance 316 may comprise a length of at least about 2.5 mm, about 3 mm, about 3.1 mm, about 3.2 mm, about 3.4 mm, about 3.5 mm, or about 4 mm. In some instances, the distance 316 may comprise a length of at most about 3 mm, about 3.1 mm, about 3.2 mm, about 3.4 mm, about 3.5 mm, about 4 mm, or about 5 mm.

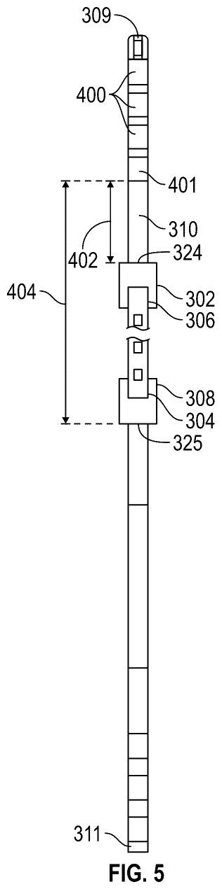

The one or more anchors of the first orientation (302, 306) and the second orientation (304, 308) may be positioned at a distance (402, 404) from the one or more electrodes 400 of the lead 310, as shown in FIG. 5. The distance 404 may comprise a length from the most proximal electrode 401 of the one or more electrodes 400 to a proximal surface of an anchor of the second orientation 325. One or more anchors may be positioned between one another by a distance of a set of up to three different distances. The one or more anchors may be releasably coupled to the lead at one or more distances from the one or more electrodes 400. A distance of an anchor from the one or more electrodes 400 may be chosen to avoid one or more target (e.g., sensitive) anatomical locations along an axis of the lead. A rotational angle of the one or more anchors coupled to the lead may be specific set to avoid one or more (e.g., sensitive) target anatomical locations along an axis of the lead. For example, the rotational angle of the barbs of one or more anchors may be adjusted to avoid the barbs from extending into and damaging sensitive tissue. The one or more anchors, described elsewhere herein, may comprise one or more barbs that do not extend and/or splay out at an angle from an axial axis of the anchor. The one or more barbs that do not extend and/or splay out may be pre-set prior to implantation to avoid a barb from extending or expanding into sensitive target tissues or anatomy surrounding the implanted lead.

The distance 404 may comprise a length of about 40 mm to about 100 mm. For example, the distance 404 may comprise a length of about 50 mm to about 100 mm, about 60 mm to about 100 mm, or about 70 mm to about 100 mm. The distance 404 may comprise a length of about 40 mm, about 45 mm, about 50 mm, about 55 mm, about 60 mm, about 70 mm, about 80 mm, about 90 mm, or about 100 mm. In some cases, the distance 404 may comprise a length of at least about 40 mm, about 45 mm, about 50 mm, about 55 mm, about 60 mm, about 70 mm, about 80 mm, or about 90 mm. In some cases, the distance 404 may comprise a length of at most about 45 mm, about 50 mm, about 55 mm, about 60 mm, about 70 mm, about 80 mm, about 90 mm, or about 100 mm.

The distance 402 may comprise a length from the most proximal electrode 401 of the one or more electrodes 400 to a distal surface of an anchor of the first orientation 324. The distance 402 may comprise a length of about 5 mm to about 12 mm. The distance 402 may comprise a length of about 6 mm to about 12 mm, or about 8 mm to about 12 mm. The distance 402 may comprise a length of about 5 mm, about 6 mm, about 7 mm, about 8 mm, about 9 mm, about 10 mm, about 11 mm, or about 12 mm. In some cases, the distance 402 may comprise a length of at least about 5 mm, about 6 mm, about 7 mm, about 8 mm, about 9 mm, about 10 mm, or about 11 mm. In some cases, the distance 402 may comprise a length of at most about 6 mm, about 7 mm, about 8 mm, about 9 mm, about 10 mm, about 11 mm, or about 12 mm.

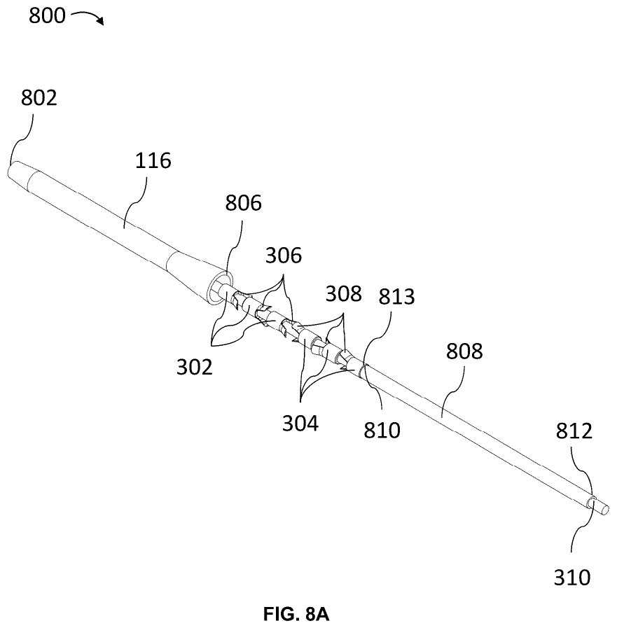

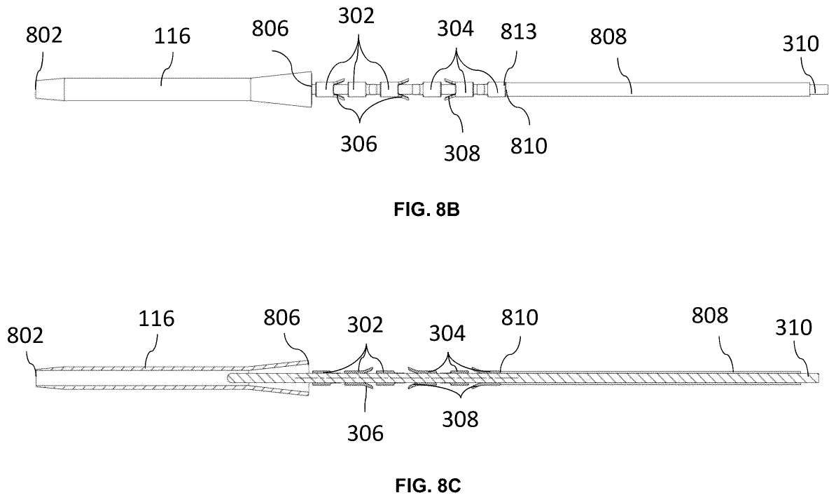

FIGS. 8A-8C, 9A-9C, and 10A-10C show an embodiment 800 of the electrode lead 310, introducer elongated body 116, pusher 808, and one or more anchors (302, 304, 306, 308), at various points in operation or points in time of using the aforementioned components as they interact with one another. A geometry of a lumen of the introducer elongated body 116 may comprise a varying geometry from a first end to a second end of the elongated body 116. The geometry of the lumen of the introducer body 116 may comprise a taper lumen geometry from a first diameter 806 of the introducer elongated body lumen to a second diameter 802 of the introducer elongated body lumen. The taper lumen geometry may comprise a funnel configured to facilitate the introduction of the barbs into the introducer elongated body lumen. For example, the taper lumen geometry from first diameter 806 to second diameter 802 may facilitate the compression of the barbs of the anchors during insertion. The taper may comprise one or more sections of tapering geometries, which may be the same or differ in length and/or diameter. The geometry of the barbs of the anchors (306, 308), described elsewhere herein, may reduce and/or minimize a force acted upon the barbs of the anchor (306, 308) by the inner lumen of the introducer elongated body as the one or more anchors (302, 304, 306, 308) on the lead 310 are pushed by the pusher 808 through the elongated body lumen. The pusher 808 may comprise an elongated body with a lumen configured to receive an outer diameter of the electrode lead. The pusher 808 may comprise a distal surface 810 configured to contact a proximal surface 813 of an anchor of a second orientation (304, 308). The pusher may push the one or more anchor (302, 304, 306, 308) and/or lead into the introducer elongated body lumen.

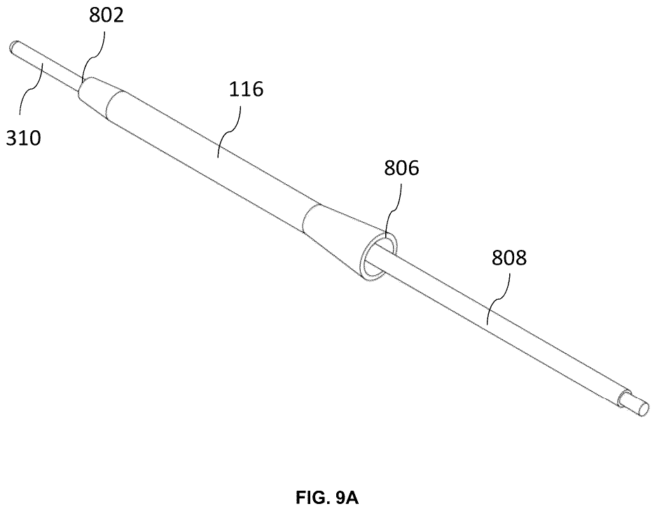

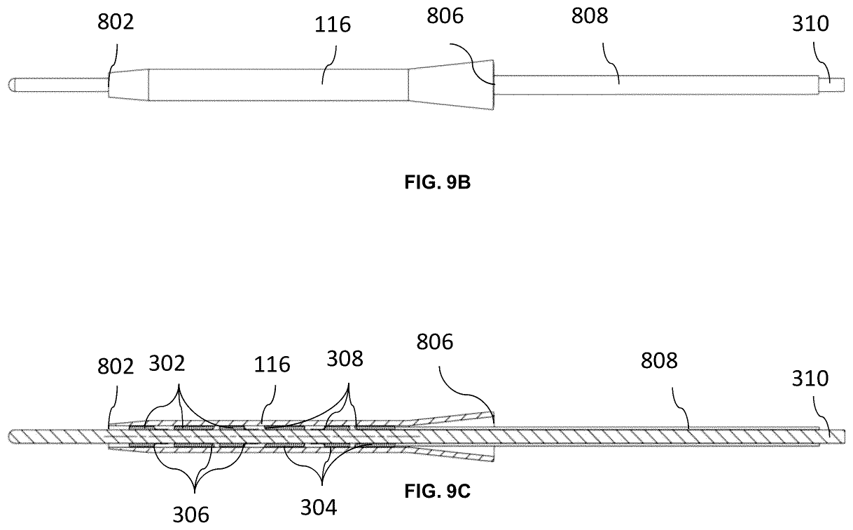

FIGS. 9A-9C, shows an embodiment of the one or more anchors (302, 304, 306, 308) on a lead 310, inserted and/or translated into the introducer elongated body 116 lumen. The sectional view of FIG. 9C shows an embodiment of the compressed and/or compacted states of the one or more anchors (302, 304, 306, 308) and pusher when inserted into the lumen of the introducer elongated body 116.

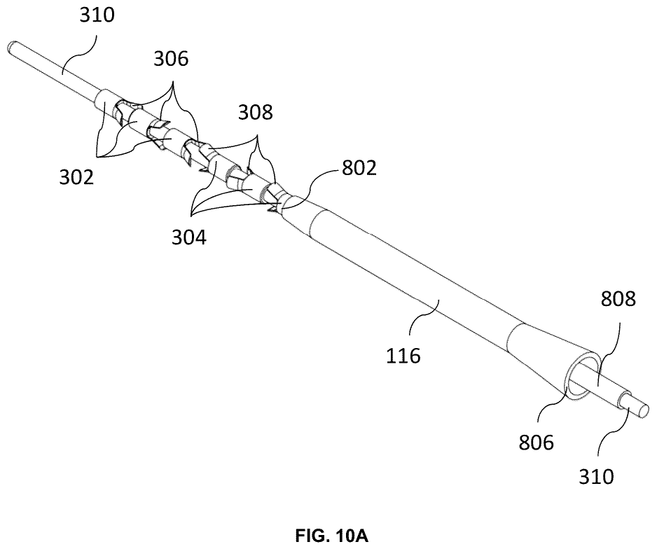

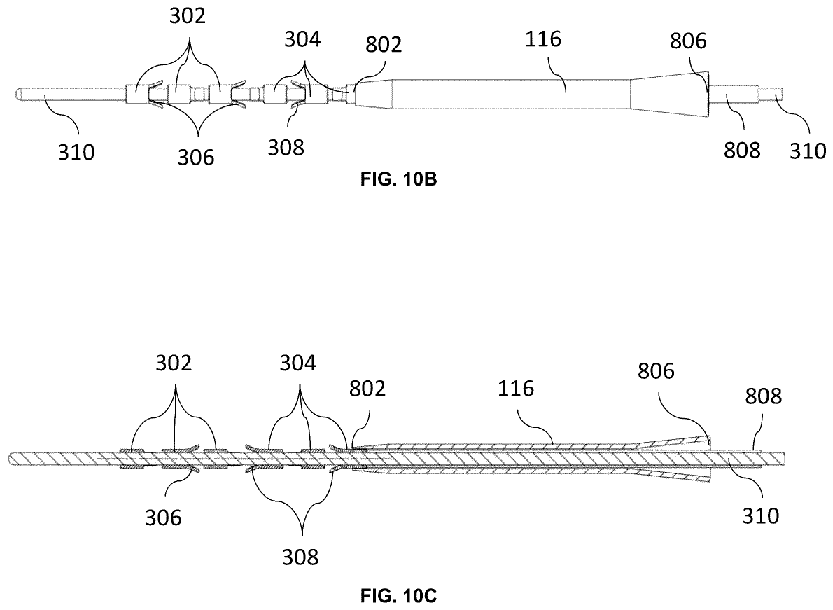

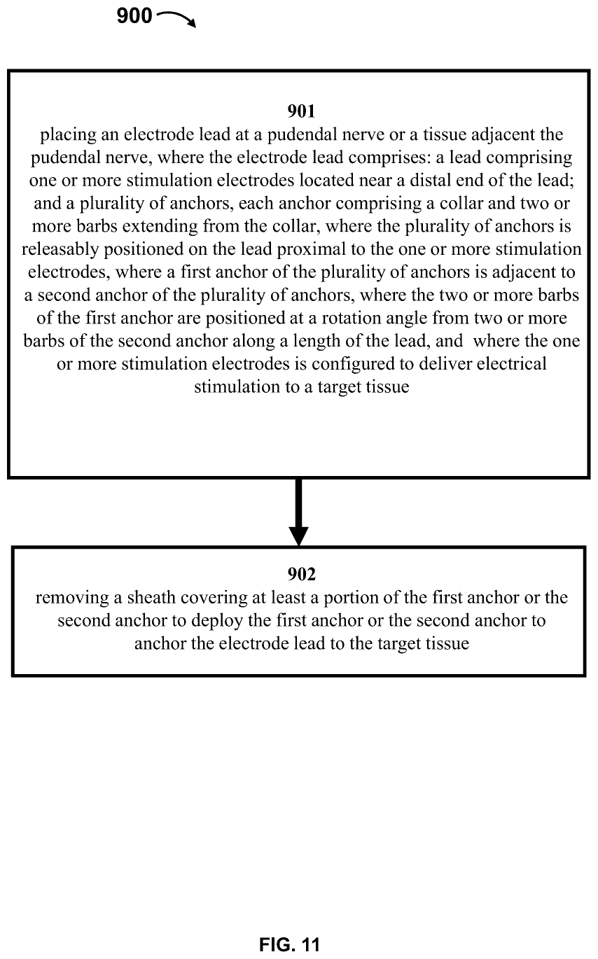

FIGS. 10A-10C, show an embodiment of the one or more anchors (302, 304, 306, 308) on a lead 310, inserted through and out of the second diameter 802 of the lumen of the introducer elongated body 116. Upon exiting the second diameter 802 of the lumen, the one or more barbs (306, 308) of the one or more anchors (302, 304, 306, 308) may expand and/or extend outward e.g., at an angle with respect to a central axis of the anchor, as shown in FIG. 10A.

### Lead Fixation Methods

Provided herein are methods for fixing the position of an electrode lead once placed at an targeted location for pudendal nerve stimulation, the method comprising (a) dilating soft tissues deep to the site of skin incision using sharp dissection or an over-sheath device that can be advanced by sliding onto the electrode lead; (b) advancing one or more anchoring devices onto the electrode lead and positioning the one or more anchoring devices into the soft tissue space created by dilatation; and (c) deploying the one or more anchoring devices such that it grips both the electrode lead and the soft tissue space thereby preventing migration of the electrode lead relative to the tissue. The method may apply to both electrode leads and sites of insertion. The sharp dissection of step (a) of the method may be completed, accomplished and/or achieved using standard surgical instrumentation e.g., a scalpel. Dilating the soft tissues of step (a) of the method may be achieved, completed, and/or accomplished by an over-sheath device to bluntly create a tunnel of a diameter sufficient to permit smooth subsequent advancement of the anchor on the electrode lead. The soft tissue deep to the site of skin incision may comprise tissue adjacent to the ischial bone and fascial and/or ligamentous insertions. The one or more anchoring devices may comprise one of a plurality of passive anchors. Advancing the one or more anchoring devices onto the electrode lead may be accomplished, completed, and/or achieved by manually advancing the one or more anchoring devices (e.g., pushing by hand or basic surgical instrument such as a clip) onto the electrode lead. In some embodiments, advancing the one or more anchoring device may be accomplished, completed, and/or achieved using an over-sheath device to advance the one or more anchoring devices into position on the electrode lead. The one or more anchoring devices may be contained within the over-sheath device before deployment. The one or more anchoring devices may be secured to the electrode lead with one or more fixation methods. The one or more anchoring devices may be secured to the soft tissue space by one or more fixation methods. The one or more anchoring devices may be fixed to the soft tissue space, where the soft tissue space may comprise native human tissues e.g., ligamentous, fascial, periosteal, or any combination thereof tissues. The one or more anchoring devices may be fixed to the soft tissue space using standard surgical approaches e.g., suturing. Physical features of the one or more anchoring devices may fix and/or secure the one or more anchoring devices to the native human tissues. The one or more anchoring devices may be fixed to the electrode lead by one or more fixation methods. The one or more anchoring devices may be fixed to the electrode lead by frictional force exerted between the one or more anchor devices and the electrode lead. In some embodiments, ligatures may be used to compress the one or more anchoring devices onto the electrode lead. Standard suture material may provide ligatures for compression of the one or more anchoring devices onto the electrode lead thereby fixing and/or securing the one or more anchoring devices to the electrode lead. In some embodiments, a small-mounted screw assembly may provide compression of the one or more anchoring devices onto the electrode thereby securing and/or fixing the one or more anchoring device onto the electrode lead. The small-mounted screw assembly may be tightened using a miniaturized torque wrench to a pre-specified pressure limit. A miniaturized torque wrench may allow tightening of the small-mounted screw assembly deep in soft tissues. In some embodiments, standard surgical ligating clips may be used to compress the one or more anchoring devices onto the electrode lead thereby securing and/or fixing the one or more anchoring devices onto the electrode lead. The standard surgical ligating clips may be applied using a ligating clip applier. The one or more anchoring devices may be fixed to the electrode lead and/or surrounding soft tissues by an activating mechanism, e.g., spring loaded tines, described elsewhere herein. The common anchoring mechanism of the one or more anchoring devices may be deployed automatically on extrusion of the one or more anchoring devices from the over-sheath device. In some embodiments, retraction of the over-sheath device may activate a common anchoring mechanism of the one or more anchoring devices. The activation of anchoring and/or fixing of the one or more anchoring devices may be activated when the one or more anchoring devices are pushed out from the over-sheath device. The one or more anchoring devices may be pushed out of the over-sheath devices by a pushing device. The one or more anchoring devices may comprise one or more mechanisms that are activated automatically when the one or more anchoring devices are extruded from the over-sheath device. The one or more mechanisms may comprise activation of spring-loaded tines of the one or more anchoring devices. The activating mechanism may comprise a clipping function of the one or more anchoring devices to the electrode lead and to the soft tissue space. Although certain devices, systems, methods, and kits are described herein with respect to the pelvic region to treat and/or manage pelvic conditions, the methods and devices can be used in other regions of the body or to treat other conditions as described elsewhere herein.

The introducer elongated body may be inserted through an incision site, described elsewhere herein, and guided to a target implantation region to implant one or more leads. A lead with one or more electrodes may be inserted into the lumen of the introducer elongated body, as shown in FIGS. 9A-9C. The lumen of the introducer elongated body may comprise a conical or tapered cross-sectional profile. In some cases, the introducer may comprise a size of about 1 French (F) to about 15 (F). Once the one or more anchors and lead are compressed and/or collapsed to a closed configuration, the introducer may then be translated and/or maneuvered to a target implantation site (e.g., at a pudendal nerve or tissue adjacent thereto). The one or more anchors (302, 304, 306, 308) and lead may then be deployed and/or expanded by translating and/or removing the introducer elongated body 116 proximally while providing a pushing force with the pusher 808 distally towards the one or more anchors and distal tip of the lead, as shown in FIGS. 10A-10C. By providing a force with the pusher 808, the position of the one or more deployed anchors (302, 304, 306, 308) may remain fixed accordingly anchoring the lead in target region. The precise positioning of the one or more anchors and lead provides targeted electrical stimulation to treat incontinence, as described elsewhere herein. The introducer elongated body may comprise a polymer, metal, or a combination thereof materials. The metal may comprise stainless steel, aluminum, titanium, or any combination thereof. The polymer material may comprise thermoplastic polyurethane (TPU), polytetrafluoroethylene (PTFE), or any combination thereof.

The introducer elongated body may comprise a length of about 140 mm to about 500 mm. For example, the introduce elongated body may comprise a length of about 150 mm to about 500 mm, about 180 mm to about 500 mm, or about 250 mm to about 500 mm. The introducer elongated body may comprise a length of about 140 mm, about 150 mm, about 160 mm, about 180 mm, about 200 mm, about 250 mm, about 300 mm, about 350 mm, about 400 mm, about 450 mm, or about 500 mm. In some cases, the introduce elongated body may comprise a length of at least about 140 mm, about 150 mm, about 160 mm, about 180 mm, about 200 mm, about 250 mm, about 300 mm, about 350 mm, about 400 mm, or about 450 mm. In some cases, the introduce elongated body may comprise a length of at most about 150 mm, about 160 mm, about 180 mm, about 200 mm, about 250 mm, about 300 mm, about 350 mm, about 400 mm, about 450 mm, or about 500 mm.

The disclosure describes a method of affixing an electrode lead to a tissue 900, as seen in FIG. 11. The method may comprise: placing an electrode lead at a target tissue, wherein the electrode lead comprises: (i) a lead comprising one or more stimulation electrodes located near a distal end of the lead; and (ii) a plurality of anchors, each anchor comprising a collar and two or more barbs extending from the collar, where the plurality of anchors is releasably positioned on the lead proximal to the one or more stimulation electrodes, wherein a first anchor of the plurality of anchors is adjacent to a second anchor of the plurality of anchors, wherein the two or more barbs of the first anchor are positioned at a rotation angle from two or more barbs of the second anchor along a length of the lead, and wherein the one or more stimulation electrodes is configured to deliver electrical stimulation to the target tissue 901; and removing a sheath covering at least a portion of the first anchor or the second anchor to deploy the first anchor or the second anchor to anchor the electrode lead to target tissue 902. The target tissue may comprise a pudendal nerve or a tissue adjacent the pudendal nerve. In some cases, the target tissue may comprise one or more branches of a pudendal nerve, sacral nerve, or any combination thereof. The sheath may comprise an introducer elongated body comprising a lumen. In some embodiments, the sheath may comprise an over sheath cover at least a portion of the first anchor and/or the second anchor. The sheath may comprise a hardness of at least about shore 75 D hardness. The sheath may be made from a material of thermoplastic polyurethane, such as Pellethane™. The sheath may be made of an aromatic polyether-based thermoplastic polyurethane (TPU), such as Techothane™. In some cases, the sheath may be made of polyether block amide, such as PEBAX™.

### Lead Positioning Guide (LPG)

Provided herein are devices, systems, and methods for introducing and placing one or more electrode leads at or near one or more target tissues. The one or more target tissues may be within the pelvic region. The target tissue comprises a pudendal nerve. In some cases, the target tissue comprises one or more branches of a pudendal nerve, a sacral nerve, or any combination thereof. The target tissue may comprise a target tissue to receive electrical stimulation for treating incontinence. In some cases, the target tissue may comprise a target tissue to receive electrical stimulation for sexual dysfunction. In some instances, the target tissue may comprise a target tissue to receive electrical stimulation for pain treatment and/or management.

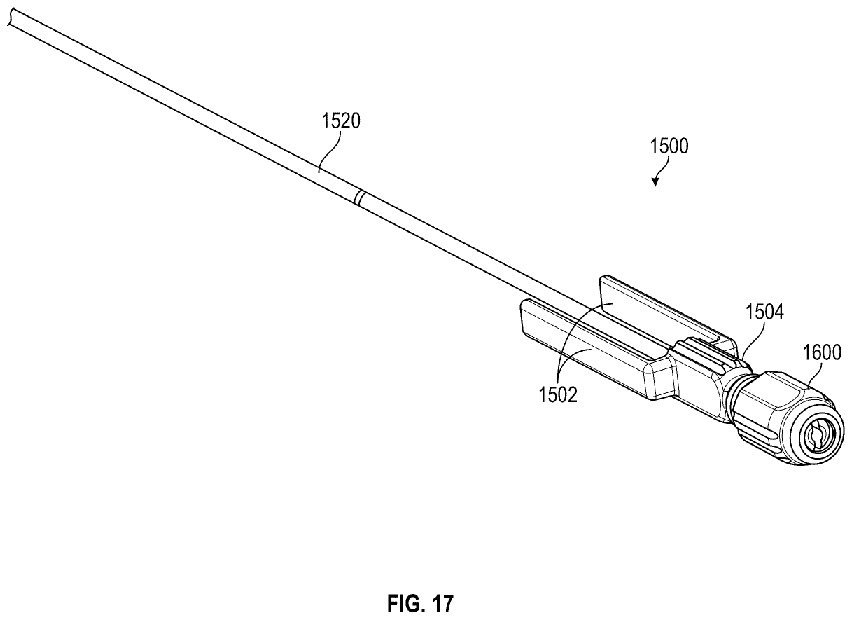

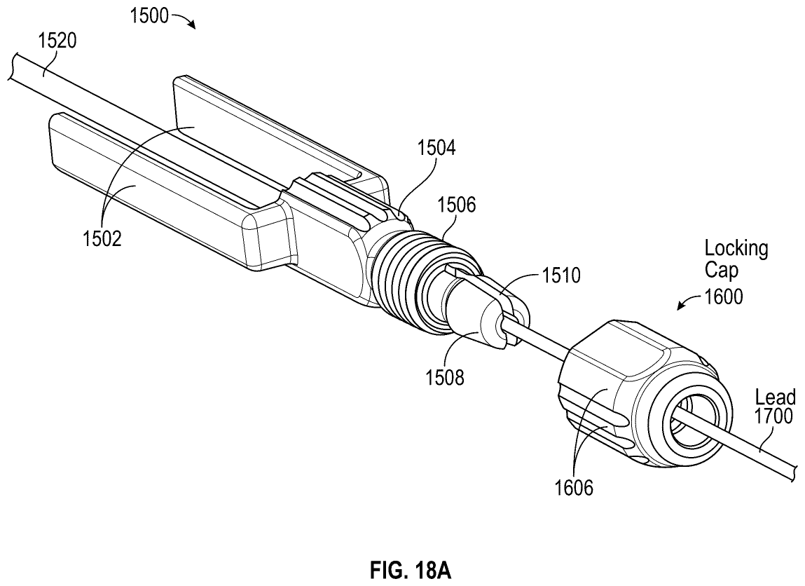

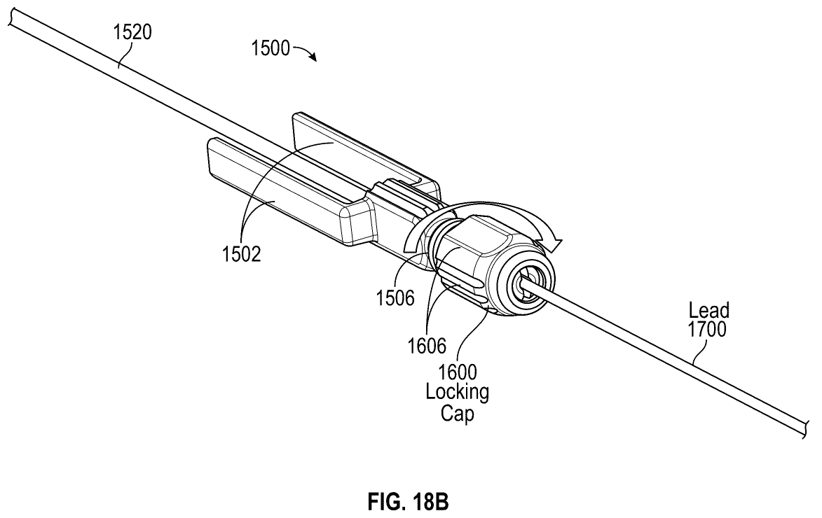

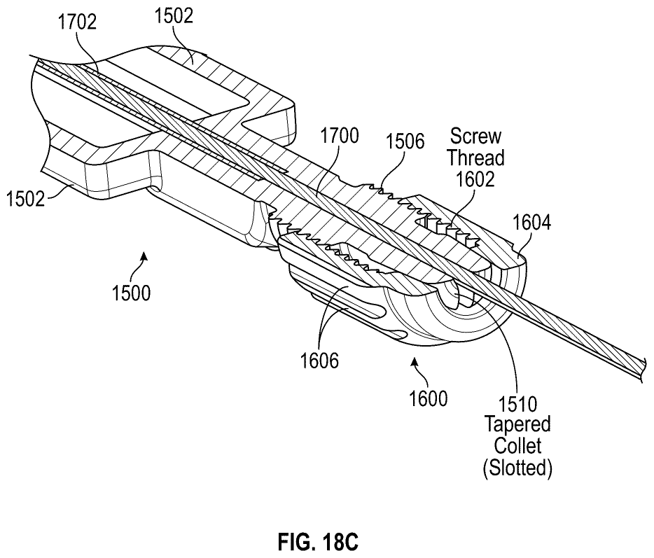

A lead positioning guide (LPG), also referred to as an anchor positioning guide (APG), used for positioning a lead comprising anchoring device may comprise a LPG 1500, a LPG tube 1520 (also referred to as a sleeve throughout the specification sleeve), and a locking cap 1600. The LPG may be used along with an introducer, as described elsewhere herein, to position one or more electrode leads at or near one or more target tissues. However, in some embodiments, one or more electrode leads may be positioned at or near one or more target tissues using only the introducer without the LPG. Although certain devices, systems, methods, and kits including the LPG are described herein with respect to the pelvic region to treat and/or manage pelvic conditions, the methods and devices can be used in other regions of the body or to treat other conditions. For example, the LPG may be used for positioning a lead at the spinal region for treating pain, or at the cranial region for treating migraines, etc. The LPG 1500 may comprise a frame comprising a proximal tip 1508, a neck portion 1504, and two or more arms 1502 extending distally from the neck 1504. The neck 1504 may comprise a screw threaded portion 1506, configured to receive a threaded portion 1602 of the locking cap 1600. FIG. 17 shows an embodiment of a LPG 1500 and locking cap 1600. FIGS. 18A-18D illustrate embodiments of a LPG 1500. The LPG 1500 may comprise a proximal tip 1508. The proximal tip 1508 may comprise an opening 1510 configured to receive a lead. The opening may extend into a lumen that extends through a neck 1504 of the LPG 1500. The neck 1504 may comprise a screw threaded portion 1506 configured to receive a locking cap 1600. The proximal tip may comprise the chafer tip. The opening 1510 may be a tapered opening. The tapered opening 1510 may be a tapered slotted opening. The slotted opening may comprise a split extending through at least a portion of the proximal tip 1508. The split may stop before a threaded portion 1506 of the neck 1504. The slotted opening 1510 may comprise two or more slits. The slotted opening 1510 may comprise three slits or four slits. The slotted opening 1510 may comprise 3, 4, 5, 6, 7, 8, 9, or 10 slits. The opening may transition into a lumen extending through the neck 1504 of the LPG 1500. The proximal tip 1508 may be tapered. The LPG 1500 may comprise a transition section between the proximal tip 1508 and the threaded portion 1506. The LPG may comprise a transition section between the threaded portion 1506 and the rest of the neck 1504. The LPG comprises a flange. The flange may match to the hub. The flange may comprise a small indent to match the hub. Matching of the flange and the hub may provide rotational stability to the components of the LPG. Matching of the flange and the hub may reduce rotational movement of the LPG components relative to each other.

The LPG system as described herein may allow for a clinician to control and maneuver a lead and anchoring device without inadvertent axial migration of the lead and anchoring device. The use of the LPG may allow a user to control and position a lead and anchoring device with one hand. The use of an LPG may allow for a user to maintain a position of a lead prior to deployment of anchor without the need to hold the lead. This may allow a clinician to release a lead during placement without a corresponding anchoring device inadvertently deploying from a delivery sleeve. The LPG may enable the controlled withdrawal of an introducer in a plurality of stages corresponding to key positions of the lead electrodes and anchors, while keeping the lead stable.

As illustrated in FIGS. 18A-18C, the locking cap 1600 may be formed as a cylindrical tube comprising a screw threaded interior 1602. The locking cap 1600 may screw onto the screw threaded portion 1506 of the neck 1504. The locking cap 1600 may extend over all or part of the proximal tip 1508. The locking cap 1600 is configured to compress the proximal tip 1508 against a lead 1700 within the slotted opening 1510 to lock a position of the lead 1700. The locking cap 1600 may be tightened or loosened around the proximal tip 1508 to control the axial moveability of the lead 1700 through the LPG 1500. The locking cap may comprise an upper ridge 1604 on the interior surface of the cap, wherein the upper ridge 1604 is configured to tighten the slotted opening 1510 around a lead 1700 when the locking cap is screwed onto the screw threaded portion 1506. The locking cap 1600 may comprise an outer gripping surface 1606 configured to assist in tightening and/or loosening the locking cap 1600 to control movement of the lead 1700. The gripping surface 1606 may comprise a material with a high friction coefficient. The gripping surface 1606 may comprise one or more surface structures to improve grip and traction on the locking cap (e.g., grooves, bumps, ridges, or the like). The locking cap 1600 may be configured to lock onto proximal tip 1508 of the LPG 1500 using a non-threaded coupler. For example, the locking cap 1600 may be configured to snap into a locked positions on the proximal tip 1508 or the next 1504 or use a lock and pin configuration. The locking cap 1600 may be configured to adjust a pressure on the lead 1700 placed through the opening 1510. The locking cap 1600 may be manually loosened and/or tightened to control the moveability of the lead 1700 during positioning. The locking cap may allow a user to vary the resistance of the lead through the LPG 1500. This may allow for better control when positioning a lead to a target position.

The LPG 1500 may comprise a LPG sleeve 1520. The sleeve 1520 may comprise an elongated body with a lumen configured to receive a lead. A proximal end of the elongated body of the LPG sleeve 1520 may be attached, fastened, and/or fused to the neck 1504 of the LPG 1500, as shown in FIGS. 18A-18C. The sleeve 1520 may comprise one or more electrodes and/or conductive regions and/or one or more electrode insulation regions. The sleeve 1520 may comprise a nonconductive biocompatible material, including but not limited to high density polyethylene, fluorinated ethylene propylene, poly carbonate, plastics, or any combination thereof. The LPG 1500 and locking 1600 may be autoclavable and/or may be cleaned by conventional sterilization methods used for other similar medical devices. In some embodiments, the sleeve may comprise one or more sensors. For example, the one or more sensors integrated into the sleeve 1520 may include but are not limited to an electrical sensor, pressure sensor, gyroscope, chemical sensor, moisture sensor, accelerometer, or any combination thereof. The sleeve 1520 may extend from a distal end of the neck 1508 between the arms 1502 of the LPG. The sleeve 1520 may be configured to receive a lead 1700 extending through the slotted opening 1510. The lead 1700 may comprise one or more anchors 1710. The anchors 1710 may comprise one or more anchors as described herein. The LPG tube 1520 may be configured to maintain the one or more anchors on a lead in a delivery configuration while positioned within the sleeve. The one or more anchors may change from a delivery configuration to a deployed configuration when extended past a distal opening of the sleeve. The sleeve 1520 may comprise an inner lumen. The inner lumen may comprise an inner diameter. The inner lumen of the sleeve 1520 may comprise a diameter such that lead 1700 and the inner lumen of the sleeve 1520 mechanically couple with a slip fit interface. In some embodiments, the inner lumen of the sleeve 1520 may comprise a diameter such that lead 1700 and the inner lumen of the sleeve 1520 have a loose fit interface. In some embodiments, the inner lumen of the introducer 111 may comprise a diameter such that outer diameter of the LPG sleeve 1520 and the inner lumen of the introducer mechanically couple with a slip fit interface. In some embodiments, the inner lumen of the introducer 111 may comprise a diameter such that outer diameter of the sleeve 1520 and the inner lumen of the introduce have a loose fit interface.

The inner lumen of the introducer 111 may comprise a diameter of about 0.2 mm to about 1.4 mm. For example, the inner lumen of the introducer 111 may comprise a diameter of about 0.3 mm to about 1.4 mm, or about 0.5 mm to about 1.4 mm. The inner lumen of the introducer 111 may comprise a diameter of about 0.2 mm, about 0.3 mm, about 0.4 mm, about 0.5 mm, about 0.6 mm, about 0.7 mm, about 0.8 mm, about 0.9 mm, about 1 mm, about 1.2 mm, or about 1.4 mm. In some cases, the inner lumen of the introducer 111 may comprise a diameter of at least about 0.2 mm, about 0.3 mm, about 0.4 mm, about 0.5 mm, about 0.6 mm, about 0.7 mm, about 0.8 mm, about 0.9 mm, about 1 mm, or about 1.2 mm. In some cases, the inner lumen of the introducer 111 may comprise a diameter of at most about 0.3 mm, about 0.4 mm, about 0.5 mm, about 0.6 mm, about 0.7 mm, about 0.8 mm, about 0.9 mm, about 1 mm, about 1.2 mm, or about 1.4 mm. A diameter may be measured by distance of the longest cross-sectional axis. In some cases, a diameter is measured by distance of the shorter cross-sectional axis (e.g., minor axis of an ellipse).

The inner lumen of the introducer 111 may comprise a diameter of about 1 mm to about 10 mm. For example, the inner lumen of the introducer 111 may comprise a diameter of about 1.5 mm to about 10 mm, about 2 mm to about 10 mm, or about 3 mm to about 10 mm. The inner lumen of the introducer 111 may comprise a diameter of about 1 mm, about 1.5 mm, about 2 mm, about 2.5 mm, about 3 mm, about 3.5 mm, about 4 mm, about 4.5 mm, about 5 mm, about 6 mm, about 7 mm, about 8 mm, about 9 mm, or about 10 mm. In some cases, the inner lumen of the introducer 111 may comprise a diameter of at least about 1 mm, about 1.5 mm, about 2 mm, about 2.5 mm, about 3 mm, about 3.5 mm, about 4 mm, about 4.5 mm, about 5 mm, about 6 mm, about 7 mm, or about 8 mm. In some cases, the inner lumen of the introducer 111 may comprise a diameter of at most about 1.5 mm, about 2 mm, about 2.5 mm, about 3 mm, about 3.5 mm, about 4 mm, about 4.5 mm, about 5 mm, about 6 mm, about 7 mm, about 8 mm, about 9 mm, or about 10 mm. The inner lumen of the introducer 111 may comprise a diameter of about 2.2±0.025 mm. The sleeve 1520 may be configured to be inserted into the inner diameter of the introducer 111. A diameter may be measured by distance of the shorter cross-sectional axis (e.g., minor axis of an ellipse). In some embodiments, a diameter may be measured by distance of the longer cross-sectional axis.

The inner diameter of the inner lumen of the sleeve 1520 may comprise a diameter of about 0.2 mm to about 1.4 mm. For example, the inner diameter of the inner lumen of the sleeve 1520 may comprise a diameter of about 0.3 mm to about 1.4 mm, or about 0.5 mm to about 1.4 mm. The inner diameter of the inner lumen of the sleeve 1520 may comprise a diameter of about 0.2 mm, about 0.3 mm, about 0.4 mm, about 0.5 mm, about 0.6 mm, about 0.7 mm, about 0.8 mm, about 0.9 mm, about 1 mm, about 1.2 mm, or about 1.4 mm. In some cases, the inner diameter of the inner lumen of the sleeve 1520 may comprise a diameter of at least about 0.2 mm, about 0.3 mm, about 0.4 mm, about 0.5 mm, about 0.6 mm, about 0.7 mm, about 0.8 mm, about 0.9 mm, about 1 mm, or about 1.2 mm. In some cases, the inner diameter of the inner lumen of the sleeve 1520 may comprise a diameter of at most about 0.3 mm, about 0.4 mm, about 0.5 mm, about 0.6 mm, about 0.7 mm, about 0.8 mm, about 0.9 mm, about 1 mm, about 1.2 mm, or about 1.4 mm. A diameter may be measured by distance of the longest cross-sectional axis. In some cases, a diameter is measured by distance of the shorter cross-sectional axis (e.g., minor axis of an ellipse).

The sleeve 1520 may comprise an outer diameter configured to be inserted through the lumen of an introducer. The introducer may be an introducer as disclosed herein. The outer diameter of the sleeve 1520 may comprise a diameter of about 1 mm to about 10 mm. The elongated body outer diameter of the sleeve 1520 may comprise a diameter of about 1.5 mm to about 10 mm, about 2 mm to about 10 mm, or about 3 mm to about 10 mm. The elongated body outer diameter of the sleeve 1520 may comprise a diameter of about 1 mm, about 1.5 mm, about 2 mm, about 2.5 mm, about 3 mm, about 3.5 mm, about 4 mm, about 4.5 mm, about 5 mm, about 6 mm, about 8 mm, or about 10 mm. The elongated body outer diameter of the sleeve 1520 may comprise a diameter of at least about 1 mm, about 1.5 mm, about 2 mm, about 2.5 mm, about 3 mm, about 3.5 mm, about 4 mm, about 4.5 mm, about 5 mm, about 6 mm, or about 8 mm. The elongated body outer diameter of the sleeve 1520 may comprise a diameter of at most about 1.5 mm, about 2 mm, about 2.5 mm, about 3 mm, about 3.5 mm, about 4 mm, about 4.5 mm, about 5 mm, about 5.5 mm, about 6 mm, about 7 mm, about 8 mm, about 9 mm, or about 10 mm. The elongated body outer diameter of the sleeve 1520 may comprise a diameter of 2 ±0.025 mm. The sleeve 1520 may be configured to assist the insertion the lead 1700 into the patient/subject receiving the electrode lead implant by providing structural rigidity. A diameter may be measured by distance of the longest cross-sectional axis. In some cases, a diameter is measured by distance of the shorter cross-sectional axis (e.g., minor axis of an ellipse).

The length of the LPG sleeve 1520 may comprise a length of about 100 mm, about 110 mm, about 120 mm, about 130 mm, about 140 mm to about 500 mm. The length of the LPG sleeve 1520 may comprise a length of about 150 mm to about 500 mm, about 180 mm to about 500 mm, or about 250 mm to about 500 mm. The length of the LPG sleeve 1520 may comprise a length of about 140 mm, about 150 mm, about 160 mm, about 180 mm, about 200 mm, about 250 mm, about 300 mm, about 350 mm, about 400 mm, about 450 mm, or about 500 mm. In some cases, the length of the LPG sleeve 1520 may comprise a length of at least about 140 mm, about 150 mm, about 160 mm, about 180 mm, about 200 mm, about 250 mm, about 300 mm, about 350 mm, about 400 mm, or about 450 mm. In some cases, the length of the LPG sleeve 1520 may comprise a length of at most about 150 mm, about 160 mm, about 180 mm, about 200 mm, about 250 mm, about 300 mm, about 350 mm, about 400 mm, about 450 mm, or about 500 mm.

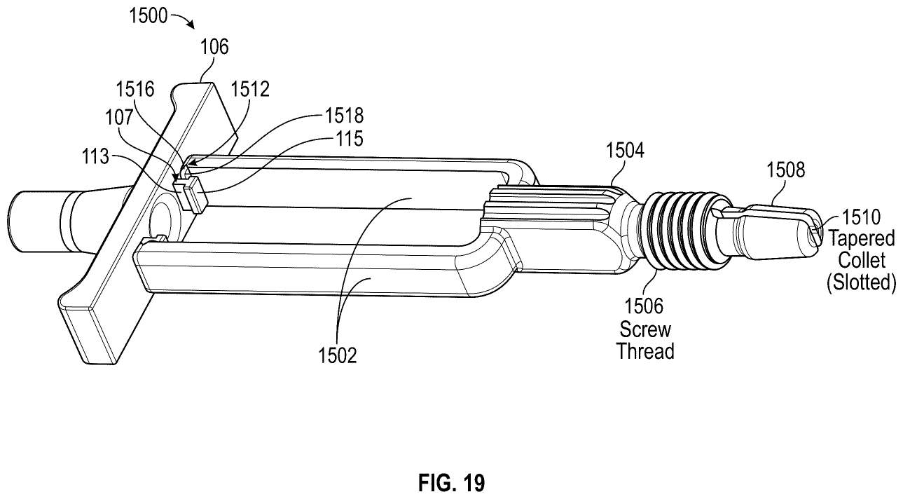

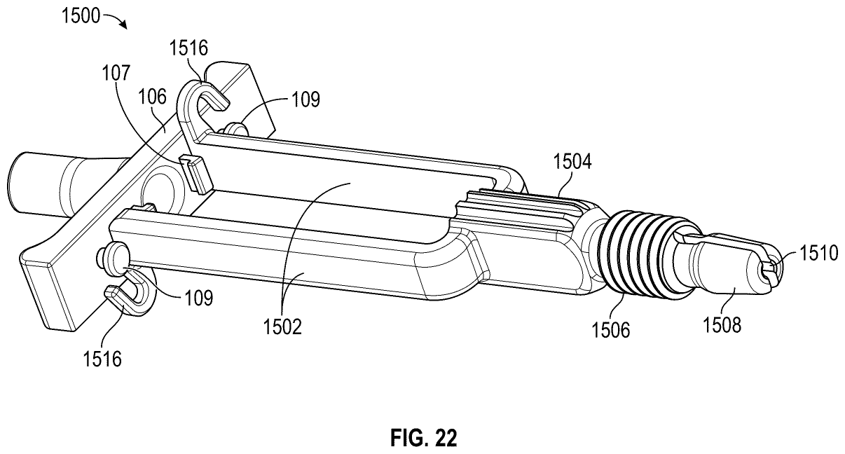

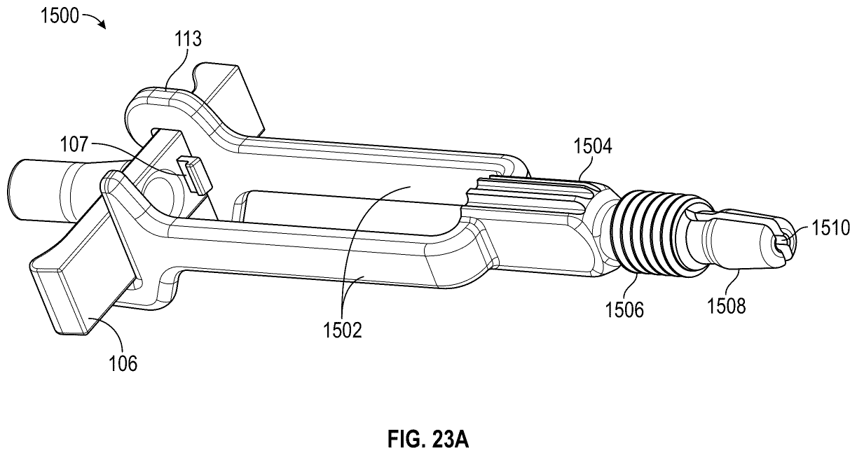

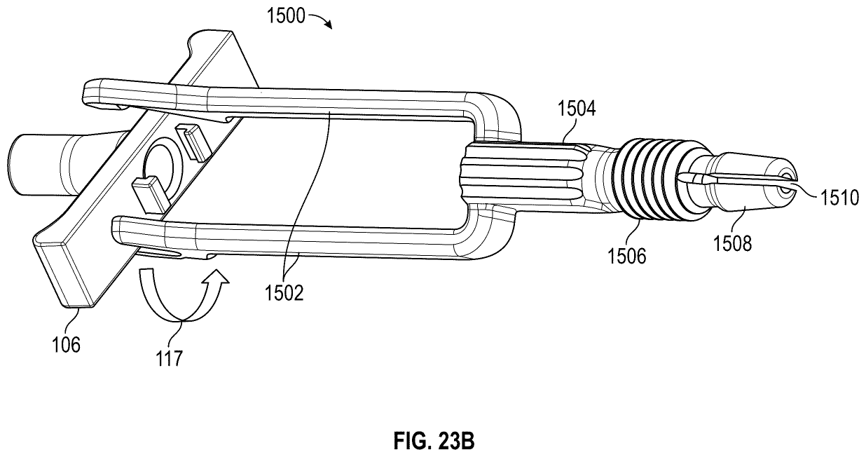

The LPG 1500 may be configured to be couple to its sheath handle 106, as shown in FIGS. 19-23. The neck 1504 may be coupled to arms 1502 of the LPG 1500. The LPG 1500 may comprise two or more arms 1502 extending from the neck 1504 of the LPG. The arms 1502 may extend distally from the neck of the LPG. The arms 1502 may extend slightly off the sides of the neck 1506 of the LPG, wherein the distance between the arms is greater than the width of the neck. The arms 1502 may comprise a coupling feature at a distal end of the arms configured to lockingly couple to a sheath handle 106. In some cases, the arms 1502 may be uncoupled from the sheath handle by rotating the LPG. The arms 1502 may comprise a locking tab 1512 configured to lockingly couple to a sheath handle 106. The locking tabs 1512 may comprise a stem 1516 and overhang 1518 configured to couple to stem 113 and overhang 115 on a sheath handle 106. The locking tabs 1512 may be lockingly coupled to receiving tabs 107 of the sheath handle 106. The sheath handle 106 may comprise receiving tabs 107 configured to mechanically clip onto locking tabs 1512. This may lock a position of the LPG in relation to the sheath handle 106. The locking tabs 1512 may comprise a stem and overhang configured to couple to stem and overhang of the receiving tabs 107. The arms 1502 may comprise an extended overhang 1514, configured to extend over a width of the sheath handle 106, as shown in FIG. 20. The overhang 1514 may comprise the locking tap configured to lockingly couple to the receiving tabs 107. The overhang 1514 may be small enough such that is does not extend over the width of the sheath handle, as shown in FIG. 19. The overhang 1514 may comprise locking tabs on two or more side of the overhang 1514. FIG. 21 illustrates an alternative embodiment, wherein a curved overhang 1514 comprise locking tabs 1512 on an inner and outer surface of the overhang 1514. The sheath handle 106 may comprise receiving tabs 107 configured to lockingly couple to the inner and outer surface locking tabs 1512 of the LPG 1500. In some cases, the LPG 1500 may comprise one or more alterative coupling features for locking the LPG to the sheath handle 106. FIG. 22 illustrates, a pin locking configuration, wherein the LPG comprise hooks/clips 1516 at the distal end of the arms 1502. The hooks/clips 1516 may be configured to lock onto pins 109 on the sheath handle 106. FIG. 23 illustrates a broad lock configuration, wherein the distal end of arms 1502 comprise a holding end. The holding ends 1518 may comprise a receiving slot configured to pinch a portion of the sheath handle 106. The receiving slot may have opposite facing openings, wherein the arms 1502 are configured to twist into place and the receiving slots pinch the sheath handle 106 when aligned with the handle 106 locking the LPG in place. The arms 1502 may be unlocked and the LPG uncoupled from the sheath handle by rotating 117 the LPG to release the sheath handle 105 from the receiving slots 1519 of the arms 1502. It should be understood that these embodiments are not limiting, and alterative locking arrangements may be employed to couple the LPG to the sheath handle.

In some cases, the sheath handle may comprise a high viscosity polyamide. For example, the sheath handle may comprise Vestamid.

The height of the receiving tabs 107 may comprise a height between about 1 mm to about 5 mm. the height of the receiving tabs 107 may comprise a height of about 2 mm to about 5 mm, or about 3 mm to about 5 mm. The height of the receiving tabs 107 may comprise a height of about 2 mm, about 2.2 mm, about 2.4 mm, about 2.6 mm, about 2.8 mm, about 3 mm, about 4 mm, or about 5 mm. The height of the receiving tabs 107 may comprise a height greater than or equal about 1.0 mm, about 1.5 mm, about 2 mm, about 2.2 mm, about 2.4 mm, about 2.6 mm, about 2.8 mm, about 3.0 mm, 3.2 mm, about 3.4 mm, about 3.6 mm, about 3.8 mm, about 4 mm, about 4.5 mm, or about 5 mm. The height of the receiving tabs 107 may comprise a height of at least about 0.5 mm, 1 mm, about 1.5 mm, about 2 mm, 2.2 mm, about 2.4 mm, about 2.6 mm, about 2.8 mm, about 3 mm, about 4 mm, or about 5 mm. The height of the receiving tabs 107 may comprise a height of about 2.4 mm.

The sleeve may comprise a hardness of at least about shore 75 D hardness. In some cases, the sleeve may comprise a hardness less than 75 D. In some cases, the sleeve may comprise prize a hardness greater than 75 D. The sleeve may comprise a hardness of about shore 75 D hardness. The sleeve may be made of thermoplastic polyurethane, such as Pellethane™. The sleeve may be made of an aromatic polyether-based thermoplastic polyurethane (TPU), such as Techothane™. In some cases, the sleeve may be made of polyether block amide, such as PEBAX™.

Provided herein are methods for positioning an electrode lead using a LPG.

Positioning an electrode lead using a LPG may comprise inserting a distal end of a lead through the proximal opening of a LPG. Positioning an electrode lead using a LPG may comprise advancing the lead through the sleeve of the LPG. The lead may be advanced through the sleeve until a distal portion of the lead extends past the distal end of the sleeve. Positioning an electrode lead using an LPG may comprise positioning one or more anchoring devices on the lead such that a proximal end of the anchor contacts the distal end of the sleeve. One or more anchoring devices may be coupled to the lead prior to advancing the lead through the sleeve of the LPG. The one or more anchoring devices may be part of the lead body. Positioning an electrode lead using a LPG may comprise placing a locking cap onto the LPG. The locking cap may restrict the axial movement of the lead within the LPG. The cap may lock the lead in a position within the LPG. Positioning an electrode lead using a LPG may comprise inserting the distal end of the lead and LPG sleeve into an introducer. The lead and LPG sleeve may be inserted into the introducer until a marker on the sleeve is aligned with the sheath handle. The introducer sheath may comprise one or more radiopaque markers that indicate a position of the electrodes and/or a position of the anchor assembly once the LPG sleeve is inserted into the introducer up to this point. For example, one or more radiopaque markers on the introducer sheath may indicate a position at or near the distal end of the sheath, as well as the proximal end, center, and/or distal end of the anchor assembly. Positioning an electrode lead using an LPG may comprise withdrawing the introducer to a marker, such that some of the electrodes are exposed and some are sheathed, e.g., two of four electrodes are exposed and the remaining two are sheathed. Positioning an electrode lead using a LPG may comprise withdrawing the introducer until the sheath handle contacts the arms of the LPG. This may cause the distal tip of the lead to be exposed, such that all of the electrodes are exposed while one or more anchors are still contained withing the introducer. Thus, the lead and LPG may be in a safe zone where the lead may still be adjusted axially while the anchors are not yet deployed from the introducer sheath. Positioning an electrode lead using an LPG may comprise locking the arms of the LPG to the handle of the introducer. The LPG handle may be locked to the handle of the introducer using one or more locking means described herein. Positioning an electrode lead using an LPG may comprise measuring EMG response and adjusting a position of the locked lead/LPG until the electrode lead receives a desired EMG signal. The EMG signal may indicate the lead is positioned at or near a target tissue. For example, an external anal sphincter EMG response may indicate that the lead is positioned at or near the pudendal nerve. Positioning an electrode lead using a LPG may comprise unlocking the LPG from the introducer handle. The LPG may be unlocked by rotating the LPG relative to the introducer handle. Positioning an electrode lead using the LPG may comprise withdrawing the introducer between the arms of the LPG and towards the proximal end of the LPG, while keeping the LPG stationary. This may deploy the one or more anchors at or near a target tissue. The one or more anchoring devices may be deployed to secure a position if the lead at or near the target site. The LPG locking cap may be unlocked, and the introducer and LPG may be withdrawn. The method may apply to leads or catheters. Methods of positioning an electrode lead using the LPG may omit and/or repeat one or more of the steps described above.

The introducer handle may comprise a high viscosity polyamide. For example, the introducer handle may comprise Vestamid.

Positioning an electrode lead using a LPG may comprise (a) inserting a distal end of a lead through the proximal opening of a LPG; (b) advancing the lead through the sleeve of the LPG until a distal portion of the lead extends past the distal end of the sleeve; (c) positioning one or more anchoring devices on the lead such that a proximal end of the anchor contacts the distal end of the sleeve; (d) placing a locking cap onto the LPG and locking the lead in position within the LPG; (e) inserting the distal end of the lead and LPG sleeve into an introducer until the sleeve marker is aligned with the sheath handle; (f) withdrawing the introducer until the sheath handle contacts the arms of the LPG, such that the distal tip of the lead is exposed and the anchors are still contained withing the introducer; (g) locking the arms of the LPG to the handle of the introducer; (h) adjusting the locked lead/LPG position until an EMG signal indicating the target tissue region is received; and (i) unlocking the LPG from the introducer handle and rotate the LPG; (j) withdrawing the introducer between the arms and towards the LPG to deploy the one or more anchors and position the lead at or near the target site. The LPG may be any LPG as described herein. In some cases, the method may apply to any lead type. In some cases, the lead may be a catheter.

A target tissue site may comprise a nerve. For example, the target tissue site may comprise the pudendal nerve. In some cases, the target tissue site may comprise the sacral nerve. The target tissue site may be targeted to help treat incontinence.

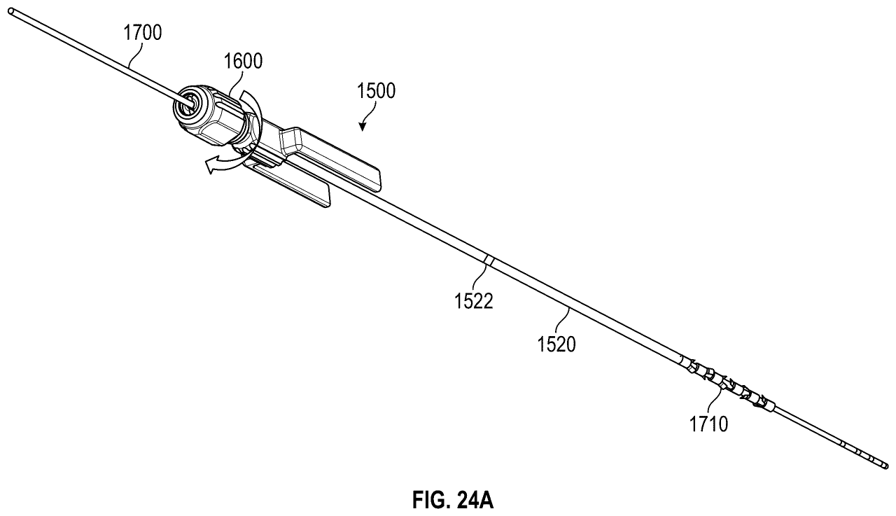

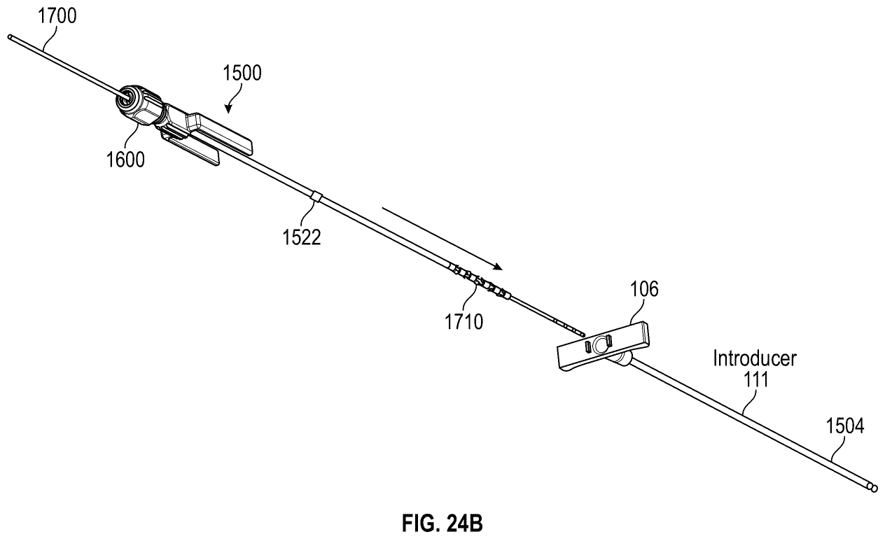

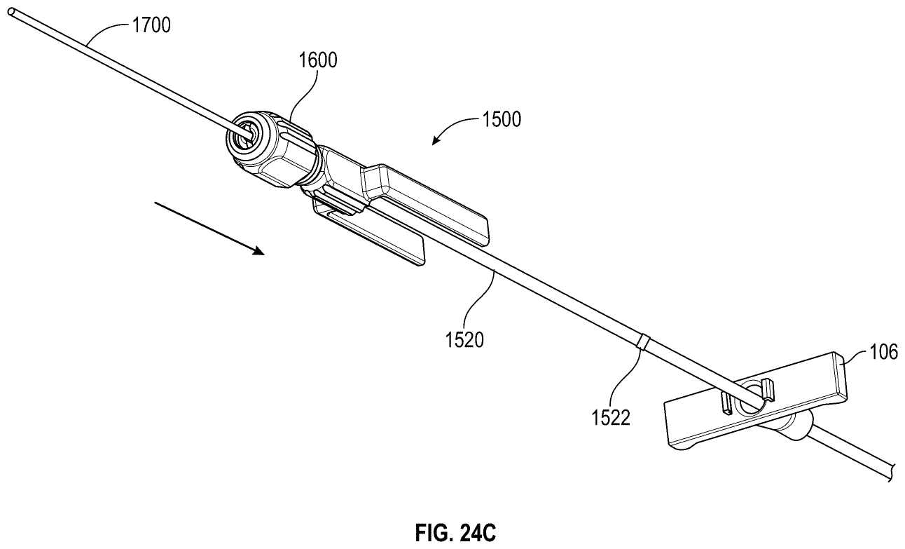

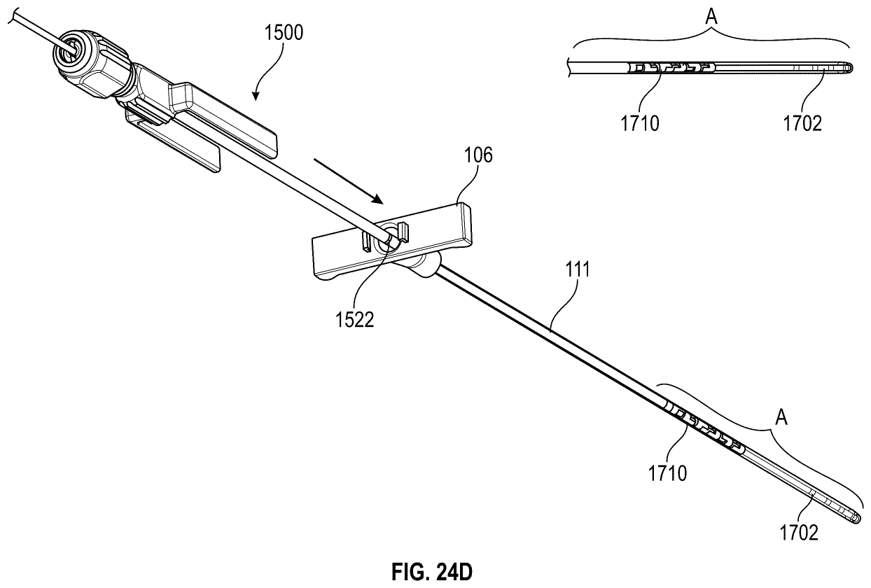

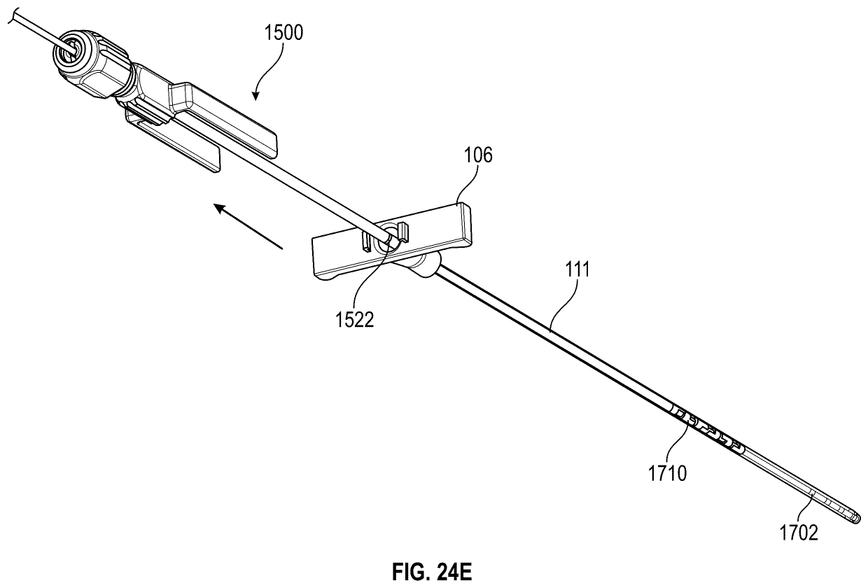

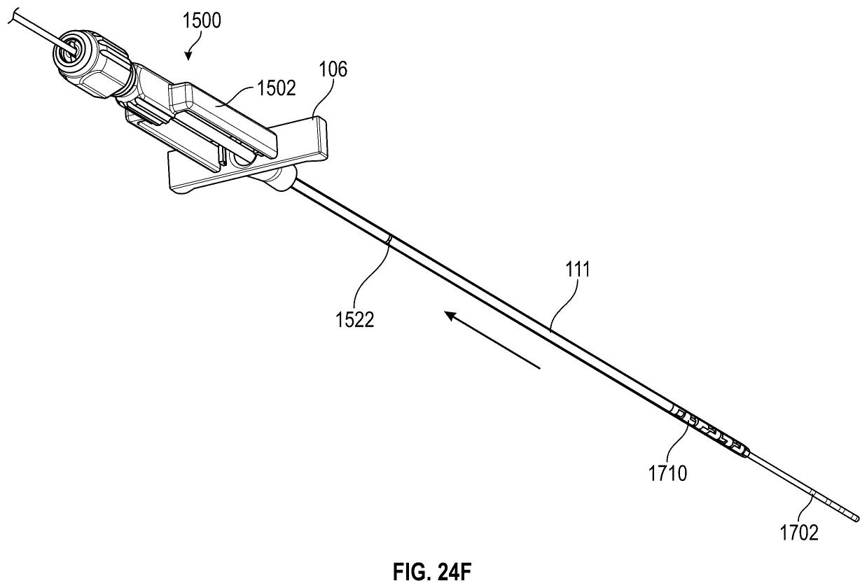

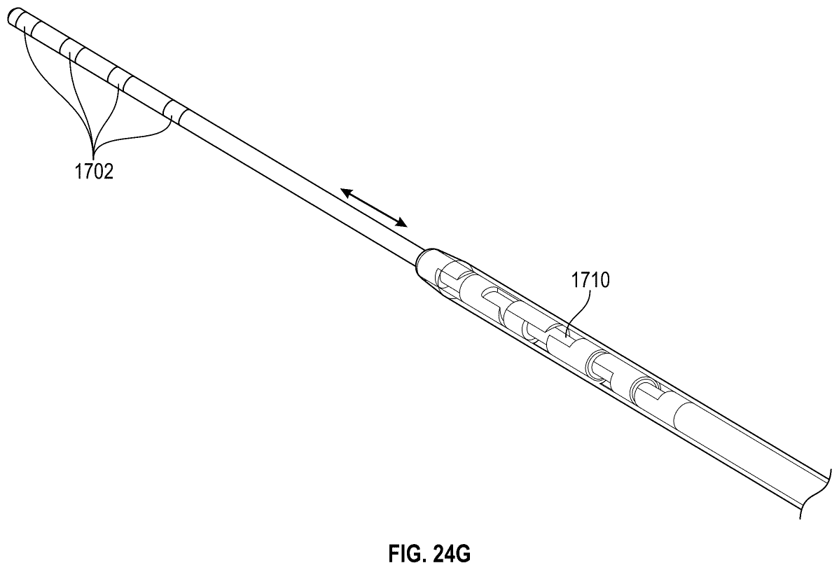

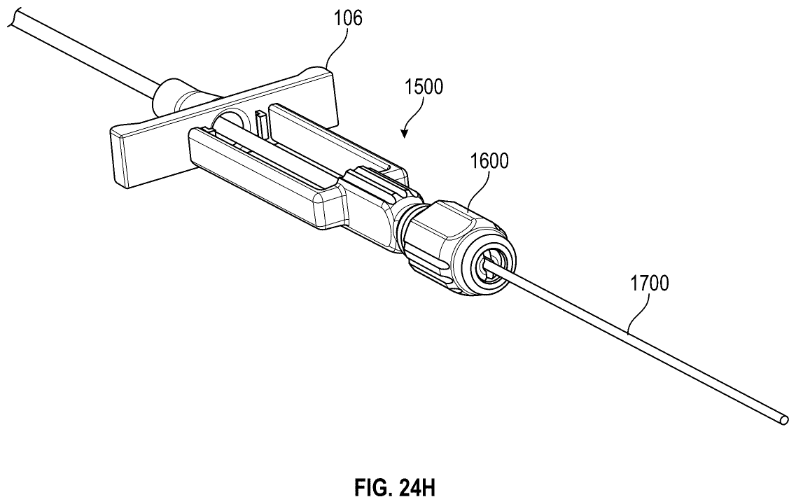

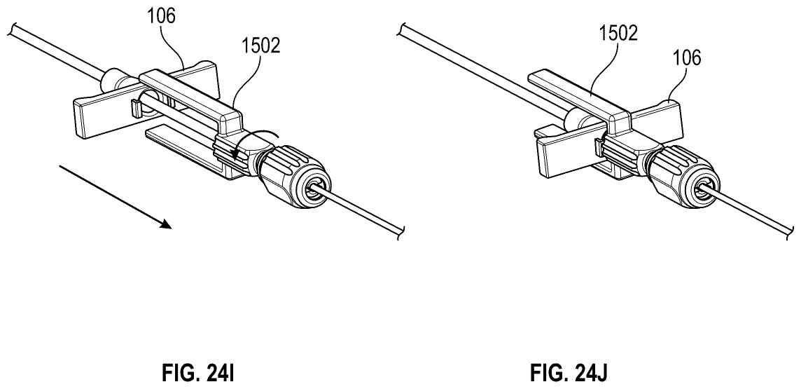

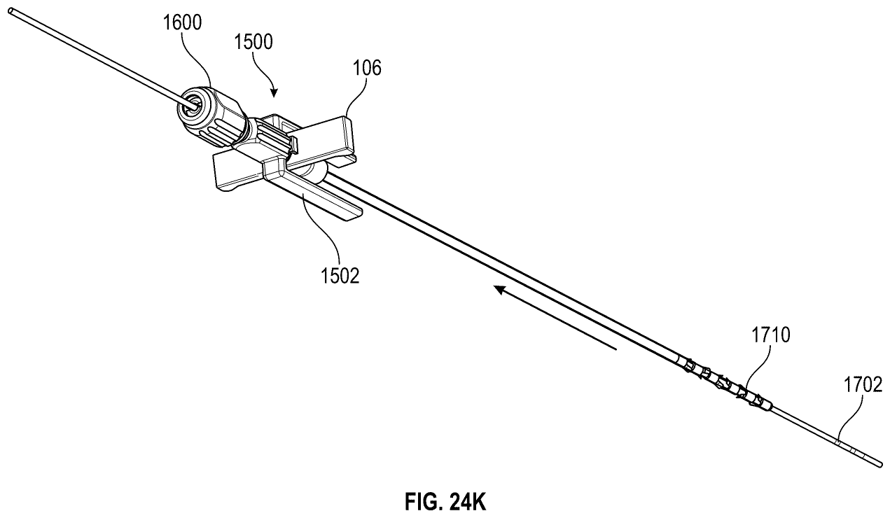

FIGS. 24A-24H illustrate an embodiment of a method for positioning and electrode lead and deploying and an anchoring device of the lead. FIG. 24A illustrates inserting a lead 1700 through the LPG 1500 and advancing the lead 1700 through the LPG sleeve 1520. Once the distal tip of the lead 1700 is extending out the distal end of the sleeve 1520 and the proximal end of the anchoring device 1710 of the lead is in contact with the distal tip of the sleeve 1520 the locking cap is screwed onto the LPG locking the position of the lead 1700 and anchoring device 1710 in position relative to the LPG 1500. FIGS. 24B and 24C illustrates inserting the locked lead 1700 and LPG sleeve 1520 through the sheath handle 106 and the introducer 111. FIG. 24D illustrates inserting the locked lead 1700 and LPG 1500 assembly through the introducer 111 until the marker on the sleeve 1520 is aligned with the opening of the sheath handle 106. One or more markers 1522 may help to indicate that the distal tip of the lead 1700 is close to the distal tip of the introducer 111. In some cases, a marker may indicate that the distal tip of the late 1700 is in line with the distal tip of the introducer 111. In some cases, a marker may indicate that the distal tip of the lead 1700 is a desired distance proximal the distal tip of the introducer 111. In some cases, a marker may indicate that the distal tip of the lead 1700 is extending a desired distance past the distal tip of the introducer 111. The one or more markers 1522 on the LPG sleeve may be radiopaque markers. In some cases, a marker 1522 may indicate the end of the sheath and proximal extent of the lead anchors 1710. The desired distance proximal the distal tip of the introducer 111 may be about 1 mm, about 2 mm, about 3 mm, about 4 mm, about 5 mm, about 6 mm, about 7 mm, 8 mm, about 9 mm, 10 mm. In some embodiments, the desired distance proximal to distal tip of the introducer 111 may be about 1 cm or greater, about 2 cm, about 3 cm, about 4 cm, about 5 cm, or about 10 cm. The desired distance past the distal tip introducer 111 may be about 1 mm, about 2 mm, about 3 mm, about 4 mm, about 5 mm, about 6 mm, about 7 mm, 8 mm, about 9 mm, 10 mm. In some embodiments, the desired distance past to distal tip of the introducer 111 may be about 1 cm or greater, about 2 cm, about 3 cm, about 4 cm, about 5 cm, or about 10 cm. The anchoring device 1710 may be still within the introducer 111, wherein the anchoring device 1700 is in a delivery configuration. FIGS. 24E-24H illustrates after aligning the marker 1522 with the opening of the sheath handle 106, withdrawing the sheath handle 106 towards the arms 1502 of the LPG 1500, wherein withdrawing the sheath handle 106 causes the arms 1502 to couple to the sheath handle. Once the sheath handle 106 is coupled to the LPG arms 1502, the LPG and lead may become locked in positions relative to the introducer 111 and sheath handle 106, as shown in FIG. 24H The sheath handle 106 and LPG 1500 may be coupled using any means of coupling or LPG configuration described above. When the sheath handle 106 contacts the arms 1502 and is coupled to the LPG 1500, the distal tip of the lead 1700 may be extended past the distal tip of the introducer 111, while the anchoring device is still positioned within a distal portion of the introducer 111 in a delivery configuration, as shown in FIG. 24G. The distal tip of the lead 1700 may comprise one or more electrodes 1702, wherein the electrodes 1702 are exposed once the sheath handle is coupled to the arms of the LPG 1502. Since the anchors are still within sheath the locked lead 1700 and LPG 1500 assembly may be free to move axially. A user may actually adjust the locked lead 1700 and LPG 1500 assembly until we receive a desired EMG response from the one or more electrodes 1702. As illustrated in FIGS. 24I and 24J, once a desired signal is received, the LPG may be rotated, wherein rotating the LPG 1500 uncouples the sheath handle 106 and LPG 1500. A user may be required to release a safety lock on the LPG 1500 and/or sheath handle 106 before the LPG 1500 may be rotated. After the LPG is rotated, the sheath handle 106 may be withdrawn between the arms 1502. This may deploy the anchoring device from the introducer 111 into a target tissue area, securing electrodes 1702 at or near a target tissue. When the sheath handle 106 is withdrawn between the arms 1502 of the LPG 1500, the anchor device 1710 may be exposed and may change from a delivery configuration to a deployed configuration as described herein. After the anchoring device is deployed, and the lead anchored in position, the locking cap may be removed and the LPG and introducer 111 may be withdrawn from the lead, as shown in FIG. 24K.

A method for positioning and electrode lead and deploying and an anchoring device of the lead may further comprise using an obturator 119. The obturator elongated body 117 may be configured to fit inside the elongated body 116 lumen of the introducer 111, as illustrated in FIG. 26. The obturator handle 104 may be configured to be couple to the introducer handle 106. The obturator elongated body 117 may comprise a tapered distal tip 2604 as illustrated in FIG. 1I. The tapered distal tip 2604 may be configured to extend distally from a distal end of the introducer 111 when inserted through the introducer. The tapered distal tip 2604 may comprise a fine, bullet tip configured to penetrate regions with high tissue density. The obturator may comprise a stiffener tube. The stiffener tube may be molded into the elongated body 117. The stiffener tube may improve stiffener and rigidity of the obturator to assist in insertion and position of the introducer 111. The obturator elongated body 117 may comprise a stiffener tube 2602 that ends before the tapered distal tip 2604 as illustrated in FIG. 1J.

The use of a LPG 1500 may allow for improved control and placement of the lead 1700 and anchoring devices 1710 to a target position. The use of the LPG 1500 may provide easier handling of the lead-in placement. The secured positioning of the and contained anchoring devices may reduce the burden of dexterity while introducing and manipulating the lead tip during placement. The use of an LPG as described herein may reduce the need for EMG and damage guidance during placement of the lead. The LPG when locking away coupled to the sheath handle may allow for precise control and positioning of one or more electrodes at or near the distal tip of the lead. The length of the arms of the LPG may allow one or more electrodes at or near the distal end of the leave to extend past the opening of the over sheath device while securely holding the lead such that it does not slidably move within the outer sheath. The arms of the LPG may be a length equal to or greater than the length of one or more anchoring devices on a lead. The length of the arms of the LPG may comprise a length greater than or equal to a length from a distal tip of a distal anchor to a proximal end of a proximal anchor. The length of the arms of the LPG may be adjustable to accommodate leads having longer or shorter distal electrode regions. The length of the arms 1502 may comprise a distance of about 20 mm to about 50 mm. For example, the length may comprise a distance of about 24 mm to about 50 mm, or about 30 mm to about 50 mm. The length may comprise a distance of about 20 mm, about 22 mm, about 24 mm, about 26 mm, about 28 mm, about 30 mm, about 40 mm, or about 50 mm. In some cases, the length may greater than or equal about 10 mm, about 15 mm, about 20 mm, about 22 mm, about 24 mm, about 26 mm, about 28 mm, about 30 mm, 32 mm, about 34 mm, about 36 mm, about 38 mm, about 40 mm, about 45 mm, or about 50 mm. In some cases, the length may comprise a distance of at least about 5 mm, 10 mm, about 15 mm, about 20 mm, 22 mm, about 24 mm, about 26 mm, about 28 mm, about 30 mm, about 40 mm, or about 50 mm. In some cases, the length of the arms 1502 may comprise a distance between about 30 mm to about 35 mm. For example, the length of the arms 1502 may comprise a distance of about 34 mm.

The LPG may be made of one or more rigid materials, for example, plastic, metal, carbon, or alloys. The opening of the LPG may be configured to accept multiple size leads. The lead may be between 1 French (F) to 18 F. In some cases, the lead may be between 1 F and 15 F. In some cases, the lead may be smaller than 6 F. In some cases, the lead may be larger than 12 F. The lead may be 1 F, 2 F, 3 F, 4 F, 5 F, 6 F, 7 F, 8 F, 9 F, 10 F, 11 F, 12 F, 13 F, 14 F, or 15 F. The locking cap may be configured to tighten the opening to accommodate different size leads. The LPG may be configured to lock the sheath handle to the neck of the LPG when in a withdrawn position. This may help prevent that introducer from sliding back down the lead. After the lead and anchoring device have been deployed the locking cap may be removed and the LPG and introducer withdrawn over the proximal portion of the lead. The arms of the LPG may be uncoupled from the sheath handle by rotating the LPG. In some cases, the arms of LPG may be uncoupled from the sheath handle by exerting an outward pressure on the arms to disengage the receiving tabs on the handle. The LPG may comprise an indicator showing when the arms are securely locked onto the sheath handle. The LPG may be locked onto the sheath handle using anyone of the locking mechanisms described herein.

The LPG sleeve may comprise a biocompatible outer lubrication layer. The LPG sleeve may comprise a low friction outer surface. Inserting the LPG sleeve through the over-sheath of the introducer may further comprise applying a lubricant. The one or more anchoring devices may comprise one or more times configured to change from a flattened delivery configuration to a deployed configuration when the introducer is withdrawn. The one or more anchoring devices may be contained within the over-sheath device before deployment. The sleeve and/or over sheath device may comprise an internal securing element configured to hold a lead in place. For example, an inflation element configured to expand inside the sleeve or sheath and hold the lead in position. One or more anchoring devices may be placed on the lead after the lead has been advanced through the sleeve. The one or more anchoring devices may be placed on the lead such that the proximal end of the anchoring device is touching the distal end of sleeve.

The distal tip of the lead may comprise one or more markers indicate when the distal portion of the lead has advanced past the distal tip of the introducer. The LPG may comprise one or more markers to assist in positioning of the lead. The LPG may comprise at least 2, 3, 4, 5, 6, 7, 8, 9, or 10 markers to assist in positions of the lead. The LPG sleeve may comprise a first marker configured to show when electrodes on the distal tip the lead extend past the distal tip of introducer. The LPG sleeve may further comprise a second marker configured to show a position right before the anchoring devices exit introducer. The combination of the first and second markers on the LPG sleeve may allow a user to track a position of the lead such that electrodes on the distal tip of the lead are extending past introducer and exposed to the tissue while the anchoring devices on the lead remain covered by the introducer and in a delivery position. A lead may comprise a marker just distal of the one or more anchoring devices to indicate that the anchors have not yet been released from the introducer. The second marker on the LPG sleeve may be configured to indicate when to stop advancing the LPG through the introducer and instead withdraw the introducer towards the arms of the LPG. The lead may comprise a marker just distal of the one or more anchoring devices to indicate that the anchors have not yet been released from the introducer. In some cases, the lead may comprise a marker just proximal of the one or more anchoring devices. The marker may indicate that the one or more anchoring devices have been deployed from the introducer. The markers on the LPG sleeve may be radiopaque markers. The radiopaque marker may indicate the end of the sheath and proximal extent of the lead anchors. The marker may be a band. The band may wrap around all or part of a circumference of the outer diameter of the sheath or sleeve. The marker may be a dot, square, arrow, triangle, or any other desired shape. The shape of the marker may be configured to help indicate a direction or orientation of the device.

A lead may comprise one or more anchoring devices prior to being inserted into the LPG and through the LPG sleeve. The one or more anchoring devices may be fixed to the electrode lead and/or surrounding soft tissues by an activating mechanism, e.g., spring loaded tines, described elsewhere herein. The common anchoring mechanism of the one or more anchoring devices may be deployed automatically on extrusion of the one or more anchoring devices from the over-sheath device. Retraction of the over-sheath device may activate a common anchoring mechanism of the one or more anchoring devices. In some embodiments, the activation of anchoring and/or fixing of the one or more anchoring devices may be activated when the one or more anchoring devices are pushed out from the over-sheath device. The one or more anchoring devices may be pushed out of the over-sheath devices by a pushing device. The one or more anchoring devices may comprise one or more mechanisms that are activated automatically when the one or more anchoring devices are extruded from the over-sheath device. In some embodiments, the one or more mechanisms may comprise activation of spring-loaded tines of the one or more anchoring devices. In some embodiments, the activating mechanism may comprise a clipping function of the one or more anchoring devices to the electrode lead and to the soft tissue space.

### Electrode Leads

Provided herein are methods, devices, and systems for implanting at least one electrode lead and an implantable pulse generator (IPG) in an individual to treat incontinence. The lead may comprise variable densities and/or flexibilities along the lead body. A portion of the lead body may be configured to have a higher rigidity to maintain its implanted position. The rigidity of a portion of the lead body may be increased by using a higher density material for that portion of the lead body. For example, the tip of the lead body may be more rigid than a turning portion of the lead. Since the tip of the lead may comprise the stimulation electrodes, the tip may have an increased rigidity and/or density such that it resists forces moving the tip away from the implanted position at or near the target nerve and lowering the effectiveness of stimulation. A portion of the lead body may be configured to have a higher flexibility to adapt to forces and strains from the movement or posture of the patient. The flexibility of a portion of the lead body may be increased by using a lower density material for that portion of the lead body. For example, a portion of the lead body that curves when a patient sits or performs other common movements may have a higher flexibility and/or lower density to adapt to strain on the lead body.

Depending on the indication, the length of the lead body may vary. In some cases, bilateral leads may have different lengths for the lead body. The length of the lead contralateral to the IPG may be greater than the length of the lead ipsilateral to the IPG. In some cases, having a bilateral approach, the difference between the length of a first lead body and a second lead body may be between about 100 mm and 200 mm. In some cases, the difference between the length of two bilateral lead bodies may be about 100 mm, about 110 mm, about 120 mm, about 130 mm, about 140 mm, about 150 mm, about 160 mm, about 170 mm, about 180 mm, about 190 mm, about 200 mm, or a length in a range defined by any of these values.

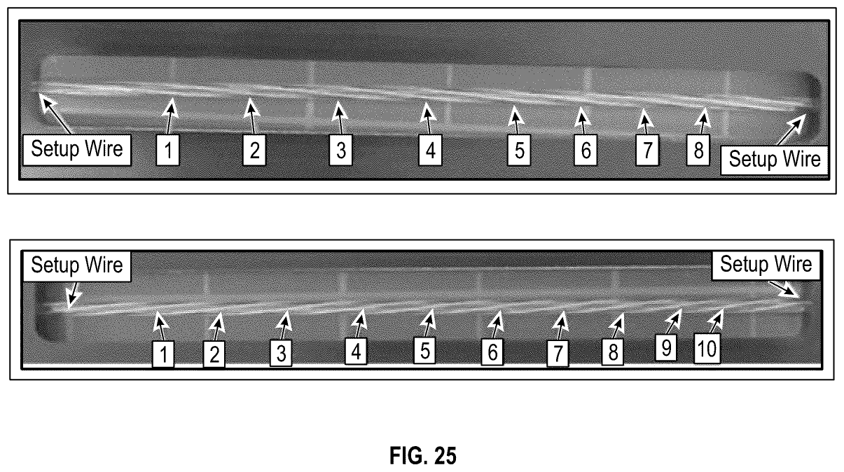

The at least one electrode lead may comprise a helical lead. The helical lead may comprise one or more cables twisted in a helical configuration as illustrated in FIG. 25. In some embodiments, only a portion of the lead may comprise one or more cables twisted in a helical configuration. The helical body of the lead may provide improved flexibility of the lead. The helical reconfiguration may help prevent the lead from kinking while advancing the lead into position. The helical reconfiguration may help prevent the lead from kinking during patient activity after implantation. The flexibility of the lead may be varied based on the twisting rate of the lead body at different parts of the lead. The helical configuration of the lead body may allow for the internal tables of the lead to extend without extended outer surface of the lead body. This may improve maneuverability around sharp turns and torturous vessels.

The one or more twisted cables may comprise a helix comprising 8±1 returns per 70 mm along the lead body. The one or more twisted cables may comprise a helix comprising 2 full rotations ±0.25 full rotations per 70 mm along the length of the lead body. In some cases, the one or more twisted cables may comprise a helix comprising 9 or more turns per 70 mm along the length of the lead body. In some cases, the one or more twisted cables may comprise a helix comprising 2.25 or more turns per 70 mm along the length of the lead body. In some cases, the one or more twisted cables may comprise a helix comprising 7 or less turns per 70 mm along the length of the lead body. In some cases, the one or more twisted cables may comprise a helix comprising 1.75 or less turns per 70 mm along the length of the lead body. In some cases, the one or more twisted cables may comprise a helix comprising 10±1.5 turns per 70 mm along the length of the lead body. In some cases, the one or more twisted cables may comprise a helix comprising 3±0.5 turns per 70 mm along the length of the lead body. In some cases, the one or more twisted cables may comprise a helix comprising 15±2 turns per 70 mm along the length of the lead body In some cases, the one or more twisted cables may comprise a helix comprising 5±0.75 turns per 70 mm along the lead body. In some cases, the one or more twisted cables may comprise a helix comprising 6±1 full rotations per 70 mm. In some cases, the one or more twisted cables may comprise a helix comprising 4±0.3 full rotations per 70 mm along the lead body. In some cases, the one or more twisted cables may comprise a helix comprising 3.5±0.25 full rotations per 70 mm along the lead body. In some cases, the one or more twisted cables may comprise a helix comprising 7±0.5 full rotations per 70 mm along the lead body. In some cases, the one or more twisted cables may comprise a helix comprising 12±1 full rotations per 70 mm along the lead body. In some cases, the one or more twisted cables may comprise a helix comprising 5±0.75 full rotations per 70 mm.

The lead may comprise one or more cables having variable pitch rates and/or coil densities along the lead body. A portion of the lead body may be configured to have a greater pitch rate or lower density at a distal end to improve its ability at the tip of the lead. One or more portions of the lead body may have a different pitch rate and/or coil density than the rest the lead body. A pitch rate of the lead may be adjusted while advancing and positioning lead. A lead may comprise a proximal portion not having a helical configuration and a distal portion having a helical configuration. In some embodiments, a lead may comprise the proximal portion having a helical configuration and a distal portion that is not helical. A proximal portion of the lead body may have a helical configuration that is less flexible than a helical configuration of a distal portion of the lead. In some embodiments, a proximal portion of the lead body may have a helical configuration that is more flexible than a helical configuration of a distal portion of the lead. In some embodiments, a lead may have a middle portion of the lead body with a helical configuration, wherein the proximal and distal ends of the lead are not helical. Any lead described herein may have a diameter of at least about 1 mm and/or less than or equal to about 10 mm, for example less than or equal to about 5 mm, or less than or equal to about 2 mm. In some cases, the helical lead may have a diameter less than 10 mm. The one or more cable may comprise platinum, gold, silver, platinum-iridium, stainless steel, MP35N, conductive metals, alloys, or a combination thereof. One or more cables of a helical lead may be platinum-iridium cables. The cable material may prevent rotation beyond a threshold number of rotations. The cable may comprise a material with a sufficient impedance value to allow for electrical stimulation. The cable may comprise a material with a sufficient impedance value to allow for receiving electrical signals. The cable may comprise a sufficiently malleable material to allow for variable coiling. The cable may comprise a material to prevent radiation past a threshold number of rotations. This may prevent short circuiting of the lead and improve safety of the device. In some cases, any lead used or described herein may comprise a helical lead.

### IPG Pocket Formation and Lead Tunneling

Provided herein are methods, devices, and systems for implanting at least one electrode lead and an implantable pulse generator (IPG) in an individual to treat incontinence. A small pocket sized to hold an IPG may be created in the ipsilateral upper buttock. The pocket may be created using a transverse skin incision. The location of the pocket may be pre-marked prior to the implantation procedure while the individual is awake. The location of the pocket may be chosen to avoid contacting clothing items, including but not limited to belts. The pocket may be close to the skin surface to allow for recharging. The IPG may be no more than about 1 cm, 2 cm, 3 cm, 4 cm, or 5 cm from the skin surface. The pocket may be no more than 2 cm from the skin surface. The pocket may be of a sufficient size to comfortably accommodate the IPG without permitting excessive migration of the IPG within the pocket, and allow the leads to exit the IPG without excessive bending stress. Care may be taken to separate the IPG from underlying gluteal muscle epimysium with a layer of fat in between the IPG and the epimysium. The IPG should be placed parallel to the skin to ensure efficient recharge.

Provided herein are methods, devices, and systems for tunneling of one or more electrode leads from the point that each lead exits the skin after lead placement, to the IPG. The tunneling may be performed to avoid penetration while achieving maximal tissue coverage in the subcutaneous fat. The tunneling may be performed with a tunneling tool. The tunneling tool may comprise a stainless steel spike and a sheath. The tunneling tool may be inserted through the gluteal fat from the skin exit point of the lead to the IPG pocket. In some embodiments having bilateral leads, the trajectory and distance of each lead tunneling path to the unilateral IPG may be different. The lead path shall be sufficiently medial to the ischial tuberosity to avoid compression of the lead by boney anatomy, e.g., during actions such as sitting & the like A crossing point of the lead contralateral to the IPG may be pre-marked by palpitation or an imaging-guided surface marking of the inferior sacroiliac joint. The tunneling path for the contralateral lead may be curved at the surface marking of the inferior sacroiliac joint, such that the path crosses the midline around the level of S3. Once the tunneling path to the IPG is created, the tunneling tool may be retracted while keeping the sheath in place. The lead may be threaded through the hollow sheath to the IPG, and the sheath may then be removed. The one or more leads may be connected to the IPG, and any excess lead may be coiled such that there may be no tension on the lead. The excess lead may be coiled beneath the IPG, away from the skin to ensure recharge is not compromised.

### Kits

The disclosure describes kits comprising the devices and components described elsewhere herein. In some cases, the kit may comprise a lead, one or more anchors, an IPG, an introducer (a) an electrode lead device, comprising: (i) a lead comprising one or more stimulation electrodes located near a distal end of the lead; and (ii) a plurality of anchors, each anchor comprising a collar and two or more barb extending from the collar, where the plurality of anchors is provided at a fixed positioned on the lead proximal to the one or more stimulation electrodes, where a first anchor of the plurality of anchors is adjacent to a second anchor of the plurality of anchors, where the two or more barbs of the first anchor are positioned at a rotation angle from two or more barbs of the second anchor along a length of the lead, and where the one or more stimulation electrodes is configured to deliver electrical stimulation to a target tissue; and (b) instructions for placing or anchoring the electrode lead to the target tissue. The kit may further comprise a LPG. The LPG may comprise a LPG frame, and LPG sleeve, and a LPG locking cap. The target tissue may comprise a pudendal nerve or a tissue adjacent the pudendal nerve. In some cases, the target tissue may comprise one or more branches of the pudendal, sacral nerve, or any combination thereof. The instructions may comprise instructions in an insert, and/or on a website (e.g., navigated to through a QR code). The kit may further comprise an introducer and/or pusher, as described elsewhere herein. The kits may comprise one or more anchors that are releasably coupled to the lead. The instructions may include directions for setting and/or modifying one or more such that a barb of the one or more barbs may be prevented and/or set to not expand when implanted in a subject.

### Methods of Manufacture

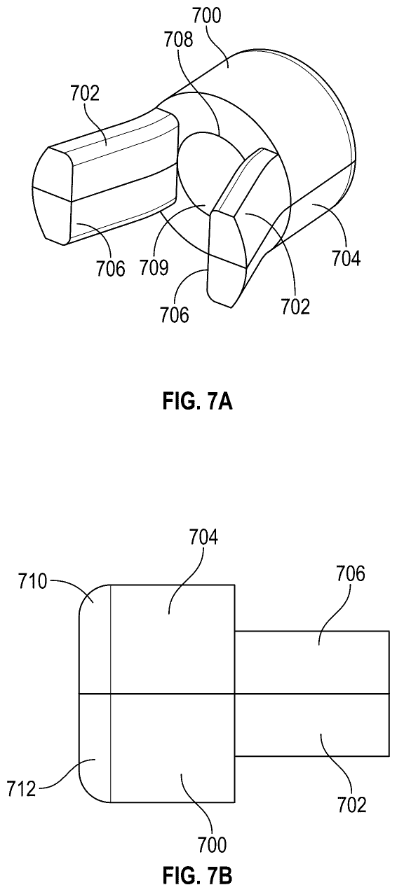

The one or more anchors, described elsewhere herein may be manufactured by molding, 3-D printing, laser-sintering, laser etching, laser cutting, or any combination thereof methods. A method of manufacturing an anchor may comprise: molding a first half (700, 702) and a second half (704, 706) of an anchor body wherein the first half (700, 702) of the anchor body comprises a first barb region 702, and the second half (704, 706) of the anchor body comprises a second barb region 706, where the first half of the anchor body is made of the same material as the first barb region, and where the second half of the anchor is made of the same material of the second barb region; and fixing the first half and the second half of the anchor body to form the anchor body. An example molded anchor may be seen in FIGS. 7A-7B. The molding may comprise injection molding. The first half (700, 702) may further comprise a first lumen 708, and the second half (704, 706) may further comprise a second lumen 709. The first lumen and the second lumen may comprise structural features of the inner diameter of the anchors, described elsewhere herein. The size and geometry of the first barb region and/or the second barb region may comprise the size, dimension, and geometry of barbs as described elsewhere herein. The first half of the anchor body and the second half of the anchor body may comprise a removably coupled feature configured to be removed and/or broken off the first half or the second half of the anchor body. The first half or the second half of the anchor body may be molded from a polymer e.g., a thermoplastic polyurethane elastomer (TPU). In some cases, the polymer may comprise polytetrafluoroethylene (PTFE). The first half or the second half of the anchor body may be molded from a material with a stiffness of at least about shore 55 D. In some cases, the first half and the second half may be fixed together with an adhesive.

The one or more anchors may be manufactured by laser cutting the one mor more anchors from a single material. The single material may comprise thermoplastic polyurethane, i.e., Pellethane™. In some instances, the single material may comprise a tube. The laser cut anchor barb may be thermally set at an angle, as described elsewhere herein, such that the barb expands to the thermally set angle when expanded and/or splayed at a target tissue. Laser cutting the one or more anchors from a single material enables complex anchor geometry and design and reduces cost of manufacturing the anchors.

A texture of one or more surfaces of the one or more anchor body or one or more barbs of the one or more anchors may be modified by manufacturing and/or processing methods described elsewhere herein. The texture of a surface of the anchor or barb may be modified and/or changed by chemical treatment, laser light, physical agitation. A change to the surface of the anchor or barb may change a coefficient of static and/or kinetic friction of the surface. A surface with an increased coefficient of friction may fix an anchor and coupled electrode lead to a target tissue with a stronger anchoring than a surface without the increased coefficient of friction.

A method of manufacturing may provide a one or more textures on one or more surfaces of the anchors described elsewhere herein. The one or more textures may comprise a first texture on the outer surface of two or more barbs of the one or more anchors, where the first texture of the outer surface facilitates or reduces forces acting on the outer surface of the two or more barbs when the barbs are inserted into an introducer elongated body lumen, as described elsewhere herein. In some instances, the one or more textures may comprise a second texture, where the second texture may increase friction or a resistance of motion between a surface of the two or more barbs of the one or more anchors and a tissue surrounding the lead and one or more anchors when implanted. The second texture may improve the robustness of fixing the implanted lead at the target anatomical region (e.g., a pudendal nerve or tissue adjacent thereto) after applying a force to the lead.

### Electrical Stimulation to Treat and Control Incontinence

Described herein are devices, systems, and methods to prevent an episode of incontinence in an individual in need thereof by providing electrical nerve stimulation. The episode of incontinence may comprise urinary incontinence, fecal incontinence, or any combination thereof. The devices, systems and methods disclosed herein may treat a sub-type of incontinence. The sub-type of incontinence may comprise urge incontinence, stress incontinence, overflow incontinence, or mixed incontinence.

Urinary incontinence may be categorized into one of four main types: urge incontinence, stress incontinence, overflow incontinence, and mixed incontinence. Urge incontinence is often due to an overactive bladder (OAB). Individuals with urge incontinence have a strong and sudden need to urinate immediately, often leaving them with insufficient time to reach a bathroom. Stress urinary incontinence (SUI) is usually due to a poorly functioning urethral sphincter muscle or hypermobility of the urethra or bladder neck. An individual may experience stress incontinence during activities such as coughing, sneezing, laughing, lifting, or exercise. Overflow incontinence may typically be due to poor bladder contraction or blockage of the urethra. Mixed urinary incontinence (MUI) may involve features of stress and urge incontinence. Incontinence often involves neurological issues, including but not limited to impaired nerve conduction between the brain and/or the affected muscles, and nervous system conditions or injuries (e.g., multiple sclerosis or stroke), or mental confusion. Other causes of incontinence include but are not limited to weakness of pelvic or urethral muscles and pelvic prolapse.

Fecal incontinence, also referred to as bowel incontinence, is the loss of bowel control, causing an individual to pass stool unexpectedly from the rectum. Fecal incontinence is usually categorized into three main types: urge incontinence, passive incontinence and post-defectory leakage (or a combination thereof). Individuals with urge incontinence have a strong and sudden need to defecate immediately, often leaving them with insufficient time to reach a bathroom. Passive fecal incontinence is when an individual passes feces without conscious awareness. Individuals suffering from passive incontinence cannot consciously control their bowel movements and stool can pass without their knowledge. Incontinence often involves neurological issues, including but not limited to impaired nerve conduction between the brain and/or the affected muscles, and nervous system conditions or injuries (e.g., multiple sclerosis or stroke), or mental confusion. Causes of fecal incontinence include but are not limited to nerve damage, anal sphincter muscle damage, constipation, diarrhea, surgery, loss of rectum storage capacity, rectal prolapse, and rectocele.

Sometimes, electrical stimulation of muscles has been used to treat incontinence by training the pelvic floor muscles thereby improving strength and function of the muscle to control over urination and defecation. In some case, electrical stimulation may target the sacral nerve to improve control over urination and defecation. In some case, the electrical stimulation approaches may benefit from stimulation of an alternate target.

The electrical stimulation approaches may be capable of delivering only a predefined stimulation protocol and may not be able to adapt to the condition and circumstances of the individual during a particular episode of incontinence. This may result in overstimulation or under stimulation of the target tissue, resulting in inadequate control over muscles involved in urination or bowel movements. Usually, traditional approaches to treating incontinence may not be able to mimic sufficiently an innate human response (i.e., reflex) to prevent an incontinence episode and may be insufficient. Individuals who have an incontinence episode may experience insufficient preventative response. The preventative response may include a muscle contraction of at least one pelvic floor muscle to prevent a leakage event in response to an increased intra-abdominal pressure. Individuals who experience stress incontinence may exhibit a delayed response in preventing an incontinence episode in response to a stress event. In some cases, individuals may experience stress incontinence related to urethral hypermobility (i.e., insufficient support) that may lead to an increased pressure transmitted to the bladder and subsequently an incontinence event.

A lack of voluntary control over micturition, defecation, incontinence, or any combination thereof is a problem that can impact quality of life and cause social embarrassment. Urinary and fecal incontinence may affect individuals of all ages. Usually, older individuals may exhibit a greater probability of incontinence with varied pathophysiology. Urinary incontinence, or loss of bladder control, and fecal incontinence, loss of control of bowel movements, often relate to neurological issues. Both urinary incontinence and fecal incontinence may involve injury, weakness, or overactivity of the pelvic floor muscles, including but not limited to the urethral and anal sphincter, and the nerves that innervate these muscles and involved organs, such as the bladder, rectum, or anus.

To treat or reduce symptoms of incontinence, electrical stimulation of the muscle, sacral nerve, and/or other pelvic nerves (e.g., the pudendal nerve) involved in incontinence has been used to improve control over micturition and bowel movements targeting the pudendal nerve, provides an improved approach to treating incontinence. Often, the pudendal nerve contributes to motor functions and mediating volitional contraction of the urethral and anal sphincter muscles in the preservation of continence. Targeting of the pudendal nerve may be combined with a closed-loop and/or feed-forward capability with dynamic adaptive control to provide a more effective treatment for incontinence. In some cases, individuals may control stimulation by a pelvic squeeze, wherein receiving a threshold EMG signal from the pelvic floor may activate electrical stimulation. However, pudendal nerve has not been a target for neuromodulation to the same degree as the sacral nerve, partly due to the difficulty in accessing and fixing leads on or close to the pudendal nerve.

Current electrical stimulation therapies may include sacral neuromodulation (SNM) that may provide fixed patterns of stimulation to treat ‘urge’ (the sudden need to urinate), but such stimulation may be unable to respond to the more common ‘stress’ incontinent events, such as coughing, sneezing, and lifting. While SNM may reduce the frequency of incontinence episodes, the success of SNM may be limited in scope (e.g., not a cure) and may decrease over time. Furthermore, SNM may not be suitable to treat individuals having stress incontinence or mixed urinary incontinence (with stress and urge incontinence). SNM may have high long-term costs in management of the electrical stimulation device and may require high level of skill and precision from the surgeon to place the lead on the sacral nerve. As such, targeting another nerve such as pudendal nerve, may provide an improved approach to treating incontinence.

Pudendal nerve stimulation (PNS) may provide a more effective treatment for individuals having incontinence than SNM. In some cases, PNS may be an effective treatment for incontinence in individuals where SNM has failed. PNS may be more powerful than SNM because the pudendal nerve enters the spinal cord through S2, S3, and S4, while SNM only acts upon the S3 root. PNS may provide an effective treatment for urinary incontinence. In some cases, the pudendal nerve may be an effective continence target for closed loop and/or feed forward stimulation.

However, the pudendal nerve has not been a target for neuromodulation to the same degree as the sacral nerve, despite the dominant role of the pudendal nerve in preservation of urinary continence. The complex 3D anatomy of the pudendal and the heterogeneity of the tissue surrounding the nerve may make the pudendal nerve a difficult target for treatment by electrical stimulation. The complex three-dimensional anatomy of the pudendal nerve may make accurate electrode placement and fixation of the implanted electrode lead more challenging. Electrophysiological guidance, rather than or in combination with radiological guidance, may be needed for accurate placement of electrodes on the pudendal nerve. There may be uncertainty regarding which section of the pudendal nerve to target (e.g., trunk vs. branches) to provide an effective electrical stimulation treatment. There may be difficulties with lead fixation due to the anatomy near the pudendal in comparison to the sacral nerve, which has a boney anatomy of sacrum to facilitate lead fixation. In some cases, there may be a concern that direct targeting of a peripheral nerve may be limited by issues of stimulation tolerability.

### Electrical Stimulation for Pain Control

The devices, systems, methods, and/or kits, described elsewhere herein, may treat pain in the pelvic region. Chronic pelvic pain (CPP) can impact quality of life, often causing episodes of severe discomfort and exacerbating pain. Common symptoms of CPP include but are not limited to neuropathic symptoms like paresthesia, numbness, burning, lancinating pain, in the pelvic, anus and/or genitals. Episodes of pain associated with CPP may frequently occur with sitting, urinating, defecating, or sexual intercourse and may be exacerbated with these activities. Approaches to treat CPP by electrically stimulating a large section of the affected area (e.g., pelvic area) or transcutaneously may have limited success at alleviating the symptoms of pain. As such, targeting specific nerves for electrical stimulation that is adapted to the individual's pain response may provide a highly efficacious treatment for CPP and other pain symptoms.

Many electrical nerve stimulation approaches currently used deliver a preset stimulation protocol (i.e., open-loop configuration) and usually are not able to adapt to the changing parameters of pain experienced by the individual. Pudendal nerve stimulation (PNS) may provide an effective treatment for chronic pain. In some cases, the inability to adapt the stimulation can result in overstimulating or under stimulating the target area and lead to inefficient or inadequate pain management. Moreover, because treatments usually do not adapt to the changing parameters of pain, existing pain management often requires patient-actuation during bouts of exacerbating pain to provide pain relief. Therefore, it would be highly beneficial to provide electrical stimulation to peripheral nerve targets that adapt to innate feedback from the subject as conditions change (e.g., a closed-loop and/or feed forward configuration).

The systems, methods, and devices, described herein are directed to treating episodes of pain associated with chronic pelvic pain (CPP) or other conditions resulting in pelvic pain using peripheral nerve stimulation. In some embodiments, the systems, methods, and devices, comprise a closed-loop and/or feed forward configuration. In some embodiments, adapted stimulation to the target nerves are provided by an implanted stimulator with an underlying physiological rationale comprising: (a) stimulating motor fibers to alter end organ muscle activity where peripheral pain is driven by spasm and/or hypertonicity (e.g., pelvic floor myalgia, some cases of bladder pain syndrome, and urethral pain associated with motor modulation); (b) stimulating larger diameter afferent fibers to modulate spinal gating of nociceptive signaling from peripheral foci of pain generation (e.g., interstitial cystitis, coccygodynia, and pelvic myalgia); (c) blocking nerve conduction (e.g., anodal block) to (i) directly block disease-related peripherally driven pain, and (ii) block noxious effects associated with providing the adapted stimulation, which facilitates higher charge delivery for therapeutic benefit; and any combination thereof.

Described herein are targeting one or more peripheral nerves based on the etiology of the pain condition with adapted electrical stimulation to reduce pain experienced by an individual. The stimulator electrodes may target different nerves (e.g., a first stimulator targeting a sacral nerve and a second stimulator targeting a pudendal nerve). In some embodiments, stimulating multiple nerves within the pelvic area may broaden the field of treatment in pain syndromes having diffuse areas of pain. In some embodiments, the electrical stimulation provided may be adapted to provide blocking and stimulation of electrical nerve signals on the same nerve. In some embodiments, stimulator electrodes may target one or more locations along a single nerve. Targeting multiple points along a nerve may allow for improved control in the closed-loop and/or feed forward modulation (e.g., a first stimulator implanted at or adjacent to a first anatomical site of a pudendal nerve and a second stimulator implanted at or adjacent to a second anatomical site of the pudendal nerve). In some embodiments, targeting a single nerve at multiple sites may permit both blocking and stimulation on the same nerve.

### Electrical Stimulation to Treat Sexual Dysfunction

The devices, systems, methods, and/or kits, described elsewhere herein, may treat sexual dysfunction of a subject. Sexual dysfunction can impact quality of life and may prevent a person from experiencing satisfaction from a sexual activity. Generally, sexual dysfunction may be classified as a desire disorder (lack of sexual desire or interest in sex), an arousal disorder (inability to become physically aroused or excited during sexual activity, an orgasm disorder (delay or absence of orgasm), or a pain disorder (pain during intercourse), or a combination of these disorders. Sexual dysfunction may involve physical and psychological causes. Often, sexual dysfunction may be treated using one or more of a medication, a mechanical aid, psychotherapy, and behavioral treatments. However, especially for sexual dysfunction with a physical cause, the effectiveness of treatment may be limited.

A number of symptoms are associated with sexual dysfunction. In both men and women, symptoms of sexual dysfunction include but are not limited to a lack of interest in or desire for sex, inability to become aroused, and pain with intercourse. In women, some symptoms associated with sexual dysfunction include but are not limited to inability to achieve orgasm, inadequate vaginal lubrication before and during intercourse, and inability to relax the pelvic floor muscles surrounding the vagina to allow intercourse. In men, some symptoms associated with sexual dysfunction include but are not limited to erectile dysfunction (ED), retarded ejaculation, and premature ejaculation. ED may refer to the inability to achieve or maintain an erection suitable for intercourse. A retarded ejaculation may refer to absent or delayed ejaculation despite sufficient sexual stimulation. A premature ejaculation may refer to an inability to control the timing of ejaculation.

ED is the most common form of sexual dysfunction in men. It is estimated that 322 million men worldwide will be affected by ED by 2025. ED has various etiologies, including but not limited to vascular, hormonal, and neurogenic causes. Individuals having neurogenic causes of ED include individuals with spinal cord injuries (SCI) and men after radical prostatectomy. Global incidence of SCI ranges from 40-80 new case per million of population per year, with 20-30 million men affected in the U.S. alone. A significant proportion of SCI individuals experiencing ED are young, where ED may significantly affect the quality of life.

To achieve an erection of the erectile tissue, contributions from both nervous and vascular components may be needed. A normal erection may rely on two reflex loops, pudendo-cavernosal reflex loop (for tumescence) and bulbocavernosus reflex loop (for rigidity). An adequate nerve activity may comprise release of nitric oxide (NO), leading to increase in cGMP, which in turn induce smooth muscle relaxation in the penile corpora. The nervous arrangement for an erection may be complex and may rely on both the somatic nervous systems (via pudendal nerve (PN)) and autonomic nervous systems (via cavernous nerve (CN)).

Usually, sexual dysfunction is treated using one or more of approaches, including but not limited to medication, mechanical aid, psychotherapy, and behavioral treatments. In some cases, the medication, also referred herein as medicament, comprises a hormone, shot, pill, or cream; or a phosphodiesterase type 5 (PDE5) inhibitor to increase blood flow to the penis; or injection papaverine, phentolamine, and/or Prostaglandin E1 (PGE1) at or near the erectile tissue. Often, PDE5 inhibitors are a first line treatment and include but are not limited to sildenafil, tadalafil, vardenafil, avanafil, lodenafil, udenafil, and mirodenafil. Although PDE 5 inhibitors are effective in treating sexual function, discontinuation rates may be high, reported as high as 50% after 1-2 years of use. Intracavernosal injections with papaverine, phentolamine, and/or PGE1 may be administered to the patient if medication is ineffective. In some cases, intracavernosal injections may result in a high incidence of adverse effects, such as priapism, injection site pain, bruising. In some cases, high incidences of adverse effects lead to patient non-compliance. Mechanical aids include but are not limited to vacuum devices, penile implants, vaginal dilators, and vibrators. In some cases, penile prosthesis implantation may result in significant complications, such as infection, erosion and pain. As such, the effectiveness of treatment may be limited, especially for sexual dysfunction with a physical etiology and when the patient compliance or response drops.

Electrical stimulation has been used as a treatment for sexual dysfunction with limited success. Sacral nerve stimulation and sacral neuromodulation has been used as a treatment for sexual dysfunction with very limited efficacy, with a reported successful intercourse in 20-30% of patients. In some cases, sacral anterior root stimulation (SARS) has been used to treat ED. However, SARS procedure may be quite invasive as rhizotomy is required and is only indicated in patients with complete SCI.

The difficulty in treating sexual dysfunction by electrical stimulation may be due to the complex organization of the nervous system involved in sexual function, such as erection, including but not limited to the pudendal and cavernous nerves. As such, electrical stimulation by electrodes at multiple nerves involved in sexual function, such as pudendal and cavernous nerves, may restore sexual function, such as erection. Electrical stimulation at both pudendal and cavernous nerves may be able to treat sexual dysfunction due to neurogenic (e.g., spinal cord injury, post-prostatectomy) or combined neurogenic/vascular etiology (e.g., diabetes mellitus, idiopathic). In some cases, the erection produced by electrostimulation may be potentiated by PDE5 inhibitor (PDE5i). In some cases, the mechanisms of action may be directly linked to PDE5i as PDE5i inhibits the degradation of the CGM by inhibiting NOS. The potential side effect of electrostimulation of somatic nerves may be reduced due to the anatomy of somatic nerves. In some cases, the potential side effect of electrostimulation of somatic nerves may be reduced as the proportion of somatic nerves surrounding the prostate apex represent <5% of the autonomic nerves.

Provided herein are devices, systems, and methods for treating a symptom of sexual dysfunction in an individual using electrical nerve stimulation. The systems, methods, and devices, described herein are directed to treating sexual dysfunction by targeted nerve peripheral stimulation and restoration and/or augmentation of reflex activity involved in sexual function. Adapted stimulation to the target nerves may be provided by an implanted stimulator with an underlying physiological rationale to target both somatic nervous system (e.g., PN) and autonomic nervous system (e.g., CN), which are involved in erection and sexual function. In some embodiments, adapted stimulation to the target nerves may be provided by an implanted stimulator to target an autonomic nerve (e.g., CN), a somatic nerve (e.g., PN), or a combination thereof. The devices, systems, and methods described herein may be configured to restore erection in case of neurogenic (e.g., spinal cord injury, post-prostatectomy) and combined neurogenic/vascular etiology (e.g., diabetes mellitus, idiopathic). In some embodiments, the systems, methods, and devices may comprise a closed-loop and/or feed forward configuration for providing the electrical stimulation.

### DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The term “in vivo” is used to describe an event that takes place in a subject's body.

The terms “approximately,” “about,” and “substantially” as used herein represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees.

As used herein, the terms “treatment” or “treating” are used in reference to an intervention regimen for obtaining beneficial or desired results in the recipient. Beneficial or desired results include but are not limited to a therapeutic benefit and/or a prophylactic benefit. A therapeutic benefit may refer to reduction, eradication, or amelioration of symptoms or of an underlying disorder being treated. Also, a therapeutic benefit can be achieved with the reduction, eradication, or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

While preferred embodiments of the present disclosure have been shown and described herein, it will be obvious to those skilled in the art that such embodiments are provided by way of example only. The phrases “in one (or some) aspects”, “in one (or some) instances”, “in one (or some) cases” shall mean “in one (or some) embodiments”. Numerous variations, changes, and substitutions will now occur to those skilled in the art without departing from the disclosure. It should be understood that various alternatives to the embodiments of the disclosure described herein may be employed in practicing the disclosure.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Disclosure of systems and methods that comprise components and steps also provides support for such systems and methods to “consist of” or “consist essentially of” those components and steps, when the latter phrases are used in the claims. As an example, disclosure herein of a kit that comprises element 1 and element 2 may include additional elements or may consist or consist essentially of element 1 and element 2.

What is claimed is:

1. A method of placing an implantable neuromodulation lead in a pelvic area of an individual, comprising:
obtaining an x-ray image of the pelvic area of the individual;
identifying one or more anatomical landmarks on the x-ray image of the pelvic area;
determining an adjustment of an orientation of an operating table based on said one or more anatomical landmarks;
adjusting the operating table based on the adjustment;
with the individual lying on the adjusted operating table, inserting a stimulation needle assembly into a body of the individual along a path to a target location at or near a nerve of the individual, the stimulation needle assembly comprising a needle having an elongate needle body,
delivering electrical stimulation from an external source via the stimulation needle assembly to tissue of the individual to elicit a response in the individual indicative of a distal tip of the stimulation needle assembly being at the target location;
advancing an introducer assembly along the path to the target location, the introducer assembly comprising an introducer sheath comprising an inner lumen;
advancing the implantable neuromodulation lead to the target location through the inner lumen of the introducer sheath.

2. The method of claim 1, wherein adjusting the operating table comprises causing an actuator to automatically adjust the operating table.

3. The method of claim 1, wherein identifying the one or more anatomical landmarks comprises identifying using a trained machine learning and/or artificial intelligence model.

4. The method of claim 1, wherein adjusting the operating table comprises lowering a left hand side, right hand side, head end, and/or foot end of the operating table.

5. The method of claim 1, wherein the one or more anatomical landmarks comprise bone landmarks associated with a pelvis.

6. The method of claim 5, wherein the bone landmarks are associated with obturator foramina of the pelvis.

7. The method of claim 1, wherein the x-ray image is in an anterior-posterior (AP) view.

8. The method of claim 1, wherein inserting the stimulation needle assembly or advancing the introducer assembly does not include use of a guidewire.

9. The method of claim 8, wherein advancing the introducer assembly comprises advancing over the elongate needle body of the stimulation needle assembly.

10. The method of claim 9, further comprising withdrawing the needle from the introducer assembly.

11. The method of claim 9, further comprising delivering the electrical stimulation from the stimulation needle assembly as the introducer assembly is advanced over the elongate needle body and into the body of the individual to verify the distal tip of the stimulation needle assembly remains at the target location.

12. The method of claim 9, wherein the elongate needle body is at least 150 mm.

13. The method of claim 9, further comprising providing the introducer assembly over the stimulation needle assembly prior to inserting the stimulation needle assembly into the body.

14. The method of claim 13, wherein the needle includes a hub at a proximal end of the needle.

15. The method of claim 13, wherein a length of the elongate needle body is greater than a length of the introducer assembly by at least 50 mm.

16. The method of claim 9, wherein the needle does not include an integral hub.

17. The method of claim 16, wherein advancing the introducer assembly comprises advancing a distal end of the introducer assembly over a proximal end of the needle.

18. The method of claim 16, wherein stimulation needle assembly further comprises a removable stylet comprising a stylet body and a stylet hub, wherein the stylet is configured to deliver the electrical stimulation.

19. The method of claim 16, wherein the stimulation needle assembly comprises a removable hub configured to be coupled to the elongate needle body and provide an electrical connection between the needle and the external source of stimulation.

20. The method of claim 19, wherein the removable hub is removed from the needle body before advancing the introducer assembly over the elongate needle body.

21. The method of claim 1, further comprising placing a guidewire through an inner lumen of the needle to the target location, and thereafter removing the needle from the body without moving the guidewire.

22. The method of claim 21, wherein advancing the introducer assembly comprises advancing over the guidewire.
